(19) United States
(12) Patent Application Publication
(10) Pub. No.: US 2026/0240771 A1
(43) Pub. Date: Aug. 20, 2026

(54) PROGRAMMABLE STIMULI-RESPONSIVE POLYMERIC FORMULATIONS

(71) Applicant: Ramot at Tel-Aviv University Ltd., Tel Aviv (IL)

(72) Inventors: Roey Jacob AMIR, Tel Aviv (IL); Amit Yehezkel SITT, Tel Aviv (IL); Nicole EDELSTEIN-PARDO, Tel Aviv (IL); Parul RATHEE, Tel Aviv (IL); Shahar TEVET, Tel Aviv (IL); Krishna VIPPALA, Tel Aviv (IL); Shira KUTCHINSKY, Tel Aviv (IL)

(21) Appl. No.: 18/992,685

(22) PCT Filed: Sep. 7, 2023
(86) PCT No.: PCT/IL2023/050970

§ 371 Date: Jan. 9, 2025

Related U.S. Application Data
(60) Provisional application No. 63/404,553, filed on Sep. 8, 2022.

(51) Int. Cl. A61K 9/107; A61K 47/32; A61K 47/34

(52) U.S. Cl. CPC A61K 9/1075; A61K 47/32; A61K 47/34

(57) ABSTRACT

The present invention provides controlled-release delivery systems composed of a formulation containing a mixture of tri- and di-block copolymers that can be programmed to undergo sequential mesophase transitions in response to multiple stimuli by tuning the molecular composition of the formulation.

### FIELD OF INVENTION

The present invention relates to stimuli-responsive polymeric formulations comprising a mixture of co-assembled tri- and di-block copolymers and methods of use thereof for controlled release active agent delivery.

### BACKGROUND

The ability of supramolecular assemblies to alter their structure and function in response to multiple stimuli in a natural environment has inspired the development of stimuli-responsive polymeric assemblies. Among the different types of stimuli, the overexpression of various disease-associated enzymes, renders them highly promising for biomedical applications. While enzymatically triggered disassembly can be applied towards selective drug release at the site of disease and clearance of the delivery system, enzymatically induced self-assembly or aggregation (EISA) can be applied towards selective accumulation of polymeric-based depots for prolonged drug release at the target site (Fakhari et al., J. Control. Release 2015, 220, 465-475). The programming of the type and sequence of these mesophase transitions is based on the incorporation of enzyme-responsive components into polymeric amphiphiles. These can include enzymatically modifiable groups, such as tyrosine and serine residues that may undergo phosphorylation or de-phosphorylation, and enzymatic cleavage sites. The enzymatic modification of the amphiphiles, which occurs at the molecular level, alters the polarity of the amphiphiles to invoke a transition into a different mesophase. Most enzyme-responsive systems contain a single type of responsive units and hence are programmed to undergo a single transition between two mesophases, such as from micelles into hydrogels and aggregates, or from soluble polymers to polymeric assemblies.

Materials that can be programmed to undergo multiple transitions between several mesophases can be extremely valuable for various applications such as controlled drug delivery (Langer et al., Nature 2004, 428(6982), 487-492; Manouras et al., Polym. Chem. 2017, 8, 74-96; Doppalapudi et al., Expert Opin. Drug Deliv. 2016, 13(6), 891-909; Cohen et al., Nat. Mater. 2010, 9(2), 101-113; Sun et al., Nanomaterials 2021, 11(3), 746). To allow both their rapid circulation in the body and selective accumulation at the site of disease, such drug delivery systems should potentially switch from stable nanostructures into soft polymeric hydrogels (Battistella et al., Adv. Mater. 2021, 33(46), 2007504). After the release of their cargo, the aggregated polymers should change their mesophase again and transform into soluble polymers that can be readily cleared from the body (Cai et al., Adv. Drug Deliv. Rev. 2022, 114463). The design of materials that can undergo multiple sequential mesophase transitions requires the incorporation of different types of responsive units into the polymeric structure (Chen et al., Adv. Mater. 2021, 33(46), 2107344). Ku et al., (JACS 2011, 133, 8392-8395) reported the ability to program materials to undergo between three mesophases by including two enzyme-responsive sites in each polymeric amphiphile.

Despite the ability to program enzyme-responsive amphiphiles to aggregate into larger structures by causing a decrease in their hydrophilic to lipophilic balance (HLB) through enzymatic degradation of the hydrophilic domains, the design of polymeric assemblies that can undergo enzymatically induced disassembly is challenging. This has been mainly attributed to the size of enzymes which is of the same order of magnitude as the polymeric assemblies. Unlike low-molecular-weight stimuli such as protons or bases (for pH responsive systems) and dimensionless stimuli such as temperature and light, the larger dimensions of enzymes can severely limit their ability to interact with the hydrophobic domains of polymeric assemblies, which are hidden and shielded by the hydrophilic shells of these assemblies (de la Rica et al., Adv. Drug Deliv. Rev. 2012, 64(11), 967-978). Kinetic studies suggest that the enzymatic degradability of the hydrophobic domains depends on the ability of the amphiphiles to escape the micelles by the unimer-assembly equilibrium as the degrading enzyme cannot penetrate the hydrophilic shells of such assemblies (Slor et al., Macromol. 2021, 54(4), 1577-1588).

WO 2016/038595 describes an enzymatic stimuli-responsive amphiphilic hybrid delivery system in micellar form, based on a hydrophilic polyethylene glycol (PEG) polymer conjugated to a hydrophobic dendron. The delivery system disassembles upon enzymatic stimuli/cleavage.

WO 2016/038596 describes an enzyme- or pH-responsive amphiphilic hybrid delivery system in micellar form for delivery of agrochemicals, based on a hydrophilic polyethylene glycol (PEG) polymer conjugated to a hydrophobic dendron. The delivery system disassembles upon enzymatic trigger or pH-based stimuli.

U.S. 2017/0035916 describes an enzymatic stimuli-responsive amphiphilic delivery system in micellar form, comprising at least one hybrid polymer or a mixture of polymers, each polymer is based on a hydrophilic polyethylene glycol (PEG) polymer conjugated to a hydrophobic dendron and a labeling moiety selected from a fluorescent dye, a dark quencher and a fluorinated moiety that acts as a magnetic probe for turn on/off of ¹⁹F-magnetic resonance (MR) signal.

U.S. 2022/0226510 describes a stimulus responsive polymer comprising: (a) a hydrophilic portion; and (b) a hydrophobic portion modified with a charged group, wherein the hydrophobic portion binds a peptide selected from the group consisting of: a cytokine, an antibody, an interleukin, and an interferon; wherein the stimulus responsive polymer is configured to release the peptide in response to a pH stimulus.

WO 2021/222523 describes a polymer comprising: a plurality of repeating units, each repeating unit comprising a polymer backbone group directly or indirectly covalently linked to one or two side chain moieties; wherein: each polymer backbone group is independently a ROMP-polymerized monomer; each one of the one or two side chain moieties independently comprises a peptide moiety or a non-peptide therapeutic moiety; wherein the polymer comprises a plurality of peptide moieties; each polymer backbone group is covalently attached to at least one other polymer backbone group; 100% of the ROMP-polymerized monomers are each individually attached to the one or two side chain moieties; and at least one side chain moiety of the polymer comprises a non-peptide therapeutic moiety, one polymer-terminating group comprises a non-peptide therapeutic moiety, and/or each of both polymer-terminating groups comprises a non-peptide therapeutic moiety.

U.S. 2020/0362095 describes triblock (ABC) bottlebrush copolymers which can be used in the formulation of particles and hydrogels for the extended release of therapeutic agents.

U.S. 2019/0117799 describes stimuli responsive amphiphilic polymers which self-assemble to form nanoparticles, wherein the stimuli are selected from the group consisting of pH, temperature, light, redox change, over-expressed enzymes, hypoxia, sound, magnetic force, electrical energy, and combinations thereof.

U.S. 2022/0202942 describes biodegradable drug delivery compositions comprising a triblock copolymer containing a polyester and a polyethylene glycol and a diblock copolymer containing a polyester and an end-capped polyethylene glycol, as well as a pharmaceutically active principle.

WO 2023/002476 describes a stimuli-induced delivery system comprising self-assembled amphiphilic tri-block copolymers in the form of micelles. The delivery system enables the release of a cargo from within the micelles following a transition to di-block copolymers micelles and subsequent activation by a protein to induce disassembly of the micelles.

There is a great unmet need for amphiphile assemblies that afford stimuli-induced controlled release of an active agent at a target site.

### SUMMARY

The present invention provides a delivery system comprising self-assembled amphiphilic polymers comprising a mixture of a di-block copolymer and a tri-block copolymer that undergo multiple mesophase transitions in response to stimuli. The delivery system of the present invention can be used for the controlled release of a cargo encapsulated within or attached thereto.

The present invention provides polymeric assemblies that can undergo programmable, on-demand transitions from nano-sized micelles to macro-scale hydrogels and subsequently to disassembled polymers. The polymeric assemblies comprise a mixture of amphiphilic di-block copolymers and amphiphilic tri-block copolymers, the amphiphilic tri-block copolymers composed of a hydrophilic segment between two substantially identical hydrophobic segments. Each of the hydrophobic segments of the di- and tri-block copolymers contain at least one stimulus-responsive cleavable site. Upon exposure to a triggering stimulus, the di-block copolymer amphiphiles disintegrate, leaving the tri-block amphiphiles substantially intact thereby inducing a mesophase transition from the nano-sized micelles into macro-scale hydrogels. The formed hydrogels undergo slower stimulus-responsive degradation to yield polymers with higher hydrophilicity, eventually leading to the complete disassembly of the hydrogels. The present invention further provides the use of the polymeric assemblies as a depot system for the controlled release of an active agent.

Disclosed herein for the first time are formulations comprising a mixture of enzyme-responsive tri-block and di-block amphiphiles which are characterized by different architectures and molecular weights, as programmable stimuli-responsive delivery systems. Surprisingly, the two amphiphiles were found to exert different kinetic stabilities thereby enabling sequential multi-step mesophase transitions induced by stimuli (e.g., activating enzymes). The mesophase transitions can be utilized for targeted delivery and formation of reservoirs (depots) of active agents at the target site thereby affording sustained release of the agents at said site. Thereafter, complete degradability can be obtained after bioactive cargo delivery.

The delivery systems disclosed herein provide multiple advantages as compared to hitherto known systems. First, the programmed mesophase transition to a hydrogel form is governed by stimulus-responsive cleavable sites at the hydrophobic segments of the di-block amphiphiles which enable better control and greater tunability over the transition as compared to systems that are based on a single polymeric component and in which aggregation into gels occurs by degradation of the hydrophilic segments of the polymers. Second, the formed hydrogel is characterized by improved kinetic stability due to the shielding of the cleavable sites located at the hydrophobic segments by the hydrophilic segment of the tri-block copolymer. In addition, the formed gel mesophase is designed to undergo further slower stimulus-responsive de-aggregation thereby affording multiple stimuli-induced sequential mesophase transitions. Finally, the amphiphiles according to the principles of the present invention are degradable enabling substantially complete clearance of the delivery system once cargo has been released.

According to a first aspect, there is provided a delivery system comprising a mixture of a di-block copolymer and a tri-block copolymer, wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween, wherein the delivery system is in the form of self-assembled micelles or fibers which transform to micelles upon hydration, and wherein the first stimulus-responsive cleavable site undergoes cleavage upon exposure to a first stimulus thereby invoking a first phase transition from micelles to a hydrogel, and the second stimulus-responsive cleavable sites undergo cleavage upon exposure to a second stimulus thereby disassembling the hydrogel and invoking a second phase transition to dissolved polymers and fragments thereof.

In some embodiments, the hydrophilic segment of the di- and/or tri-block copolymer comprises polyacrylic acid, poly(hydroxyethyl acrylate), polyethylene glycol (PEG), poly(oligo-ethylene glycol acrylate), polyacrylamide, polymethyl oxazoline, polyethyl oxazoline, polysarcosine, polypeptide, polypeptoid, hydrophilic polymethacrylate, polyamine, hydrophilic nylon, polyvinyl alcohol, hydrophilic protein, or polycarbohydrate. Each possibility represents a separate embodiment. In other embodiments, the hydrophilic segment comprises polyacrylic acid, poly(2-hydroxyethyl acrylate), polyethylene glycol (PEG), or poly(oligo-ethylene glycol acrylate). Each possibility represents a separate embodiment. In one embodiment, the hydrophilic segment comprises polyethylene glycol (PEG). In particular embodiments, the hydrophilic segment has a molecular weight of about 0.5 to about 100 kDa, including each value within the specified range. In other particular embodiments, the hydrophilic segment has a molecular weight of about 0.5 to about 50 kDa, including each value within the specified range. In yet other particular embodiments, the hydrophilic segment has a molecular weight of about 0.5 to about 25 kDa, including each value within the specified range. In one embodiment, the hydrophilic segment does not contain a peptide or a polypeptide.

In further embodiments, the hydrophilic segment is linked to the hydrophobic segment(s) by a group selected from the group consisting of —Z—X—, —X¹—Z—X²—, and —Z¹—X¹—Z²—X²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof. Each possibility represents a separate embodiment.

In other embodiments, the hydrophobic segments of the di- and/or tri-block copolymer comprise hydrophobic dendrons. In some embodiments, the hydrophobic dendrons comprise between 0 to 5 generations. In other embodiments, the hydrophobic dendrons comprise between 0 to 3 generations. In further embodiments, the hydrophobic dendrons are generation 0 (G0) dendrons. In other embodiments, the hydrophobic dendrons are generation 1 (G1) dendrons. In yet other embodiments, the hydrophobic dendrons are generation 2 (G2) dendrons. In particular embodiments, the hydrophobic dendrons are generation 3 (G3) dendrons.

In certain embodiments, each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof. Each possibility represents a separate embodiment.

In various embodiments, each generation of the dendron is derived from a compound having the following structure HX—Z—XH or HX—Z—CO₂H, wherein X is independently at each occurrence NH, S or O, and Z is selected from C₁-C₁₀ alkylene, C₂-C₁₀ alkenylene, C₂-C ₁₀ alkynylene, and arylene. Each possibility represents a separate embodiment. In other embodiments, each generation of the dendron is derived from a compound selected from the group consisting of HX—CH₂—CH₂—XH, HX—(CH₂)₁₋₃—CO₂H and HX—CH₂—CH(XH)—CH₂—XH wherein X is independently at each occurrence NH, S or O. Each possibility represents a separate embodiment. In particular embodiments, each generation of the dendron is derived from a compound selected from the group consisting of HS—CH₂—CH₂—OH, HS—(CH₂)₁₋₃—CO₂H and HS—CH₂—CH(OH)—CH₂—OH. Each possibility represents a separate embodiment. In further embodiments, where the hydrophobic segments comprise hydrophobic dendrons, the stimulus cleavable sites are present at one or more of the terminal repeating units (i.e., terminal generations) of the hydrophobic dendrons, and/or in intermediary generations of the dendrons. Each possibility represents a separate embodiment.

In various embodiments, the first and/or second stimulus is chemically-induced. In other embodiments, the first and/or second stimulus is physically-induced. In particular embodiments, the first and/or second stimulus comprises a change in at least one of temperature, pH, light (UV light, visible light or near infrared light), and electric field. Each possibility represents a separate embodiment. In other embodiments, the first and/or second stimulus comprises the addition of a redox agent.

In currently preferred embodiments, the first and/or second stimulus comprises the addition of a transport protein or an activating enzyme. Each possibility represents a separate embodiment. In one embodiment, the transport protein is a serum albumin. In another embodiment, the activating enzyme is selected from the group consisting of an amidase, an esterase, and a urease. Each possibility represents a separate embodiment. In accordance with the latter embodiments, the first and/or second stimulus-responsive cleavable site comprises an enzymatically cleavable site.

In certain embodiments, the stimulus-responsive cleavable site comprises a cleavable bond selected from the group consisting of a disulfide, a diselenide, an anhydride, an ester (including a boronate ester and a phosphate ester), an amide, an amidine, an imine, a carbamate, a carbonate, an acetal, a urea, a thiourea, a trithionate, a sulfate, a sulfamate, a phosphate, a phosphoamide, a hydrazone, an ether, a silyl ether, an oxyme, a boronic acid, a nitro, and an azo. Each possibility represents a separate embodiment. In specific embodiments, the stimulus-responsive cleavable site comprises a functional group represented by the structure of —O—C(O)—R′, —C(O)—OR′—NH—C(O)—R′ or —C(O)—NHR′, wherein R′ is C₁-C₁₂ alkyl or an aryl. Each possibility represents a separate embodiment.

In some embodiments, the delivery system comprises a mixture of a di-block copolymer and a tri-block copolymer at a mole ratio of about 1:10 to about 10:1, including all iterations of ratios within the specified range. In other embodiments, the delivery system comprises a mixture of a di-block copolymer and a tri-block copolymer at a mole ratio of about 1:5 to about 5:1, including all iterations of ratios within the specified range. In yet other embodiments, the delivery system comprises a mixture of a di-block copolymer and a tri-block copolymer at a mole ratio of about 1:3 to about 3:1, including all iterations of ratios within the specified range. In particular embodiments, the delivery system comprises a mixture of a di-block copolymer and a tri-block copolymer at a mole ratio of about 1:1. In other particular embodiments, the delivery system comprises a mixture of a di-block copolymer and a tri-block copolymer at a mole ratio of about 2:1. In yet other particular embodiments, the delivery system comprises a mixture of a di-block copolymer and a tri-block copolymer at a mole ratio of about 3:2.

In certain embodiments, the hydrophilic to lipophilic balance (HLB) of the di-block copolymer is substantially identical to the HLB of the tri-block copolymer. In particular embodiments, the di- and tri-block copolymers are synthetic. Currently preferred di- and tri-block copolymers in accordance with the principles of the present invention are represented by the structures depicted in any one of FIGS. 2A, 17, and 39. Each possibility represents a separate embodiment.

In some embodiments, the micelles have an average particle size in the nanometer range. In various embodiments, the micelles are characterized by having an average particle size of less than about 100 nm, preferably about 80 nm or lower, more preferably about 50 nm or lower, and most preferably about 5 nm to 50 nm, including each value within the specified range.

In one embodiment, the delivery system further comprises a cargo encapsulated within the micelles. In another embodiment, the delivery system further comprises a cargo covalently linked to the hydrophobic segment of the di-block copolymer. In yet another embodiments, the delivery system further comprises a cargo covalently linked to the hydrophobic segments of the tri-block copolymer. In further embodiments, the cargo is selected from the group consisting of a pharmaceutical active ingredient, an agrochemical agent, a cosmetic agent, an imaging agent, and a diagnostic agent. Each possibility represents a separate embodiment.

According to the principles of the present invention, upon activation by a first stimulus, cleavage of the first stimulus-responsive cleavable site occurs thereby disassembling the micelles and invoking a phase transition to a hydrogel. It is contemplated that when a cargo is encapsulated or attached to the tri-block copolymer amphiphiles, negligible/substantially no release of the cargo is affected by this phase transition. Thus, according to the principles of the present invention, release of the cargo is only affected by the second phase transition.

In some embodiments, cargo release is performed in a controlled manner to provide a modified release pattern. Within the scope of the present invention are sustained release depot formulations.

According to another aspect, there is provided a method of delivering a cargo to a target site, the method comprising the steps of:
(i) providing a delivery system comprising a mixture of a di-block copolymer and a tri-block copolymer, wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween, and a cargo encapsulated within or attached thereto, wherein the delivery system is in the form of self-assembled micelles or fibers which transform to micelles upon hydration;
(ii) applying a first stimulus to induce cleavage of the first stimulus-responsive cleavable site thereby invoking a first phase transition from micelles to a hydrogel; and
(iii) applying a second stimulus to induce cleavage of the second stimulus-responsive cleavable sites and disassembly of the hydrogel thereby invoking a second phase transition to dissolved polymers and fragments thereof and releasing the cargo at the target site.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 depicts a schematic illustration of various aspects and embodiments according to the principles of the present invention. A mixture of DBA and TBA hybrids are co-assembled into mixed micelles. Upon activation by a first stimulus (e.g., an activating enzyme), faster enzymatic cleavage of the hydrophobic end-groups of the DBA occurs. The degradation of the DBA leads to an increase in TBA concentration, causing the aggregation thereof into a hydrogel phase. Upon exposure of the hydrogel to a second stimulus (e.g., an activating enzyme which may be the same or different than the first activating enzyme), slow degradation of the TBA occurs which results in an additional mesophase transition into soluble polymers and fragments thereof.

FIGS. 2A-2G depict a delivery system according to embodiments of the present invention. (2A) Structures of DBA and TBA amphiphiles according to embodiments of the present invention. (2B) An image of a vial containing a clear micellar solution formed by the amphiphiles. (2C) DLS spectrum of the co-assembled micelles. (2D) A TEM image of the micellar solution. (2E) An overlay of HPLC chromatograms demonstrating faster enzymatic cleavage of the hydrophobic end groups of the DBA by a first stimulus. (2F) Kinetic data demonstrating the degradation of DBA which leads to aggregation into a hydrogel phase. (2G) An image of the hydrogel at the bottom of the vial and a schematic illustration of the slow degradation of the TBA induced by a second stimulus.

FIG. 3 depicts GPC traces overlay of commercial 5 kDa methoxy PEG, mPEG_{5kDa}-allyl, mPEG_{5kDa}-NH₂, mPEG_{5kDa}-Lys(Boc)-Fmoc, mPEG_{5kDa}-Lys(Boc)-[dend-(yne)₂], and mPEG_{5kDa}-Lys(Boc)-[dend-(hexanoate)₄].

FIG. 4 depicts GPC traces overlay of commercial 10 kDa PEG, bPEG_{10kDa}-allyl, bPEG_{10kDa}-NH₂, bPEG_{5kDa}-Lys(Boc)-Fmoc, bPEG_{5kDa}-Lys(Boc)-[dend-(yne)₂], and bPEG_{5kDa}-Lys(Boc)-[dend-(hexanoate)₄].

FIGS. 5A-5B depict DLS graphs of di-block and tri-block copolymers in micellar form at t=0. (5A) compounds (3) and (10) at a 1:1 ratio and (5B) at a 2:1 ratio.

FIGS. 6A-6B depict TEM images of di-block and tri-block copolymers in micellar form at t=0. (6A) compounds (3) and (10) at a 1:1 ratio and (6B) at a 2:1 ratio. Scale bar=50 nm.

FIG. 7 depicts images of gel formed at the bottom of a vial (left) and at the top of an inverted vial (right) of compound (10) in water.

FIG. 8 depicts the HPLC spectrum obtained after adding acetonitrile to the gel formed after the degradation of the di-block amphiphiles.

FIG. 9 depicts fluorescence spectra of micelles containing both dyes as a function of time after the addition of PLE.

FIG. 10 depicts images of the solutions at different time points.

FIG. 11 depicts images of vials containing hydrogels with BSA (left vials) and BSA and PLE (right vials) over 8 days, indicating hydrogel transformation into free polymeric chains in the presence of PLE.

FIG. 12 depicts the fluorescence spectra of micellar Nile red solution over time.

FIG. 13 depicts images of the cuvettes at different time points.

FIG. 14 depicts confocal images before (left) and after (right) PLE addition.

FIGS. 15A-15B depict images of a solution at t=0 before the addition of PLE (15A) and of the gel formed (at the bottom of the vial) after the micelles disassembly in the two ratios tested (15B).

FIGS. 16A-16B depict the enzymatic degradation of the amphiphiles at the ratio of 2:1 DBA:TBA. (16A) HPLC data and (16B) kinetic data.

FIG. 17 depicts the molecular structures of four hybrids according to embodiments of the present invention: mPEG₅ₖ-[dend-(hexanoate)₄], mPEG₅ₖ-[dend-(PhAcm)₄], bPEG₁₀ₖ-bis-Lys(Coum.)-[dend-(hexanoate)₄], and bPEG₁₀ₖ-bis-Lys(Coum.)-[dend-(PhAcm)₄].

FIGS. 18A-18E depict the mesophase transition of a delivery system according to embodiments of the present invention containing Amide-DBA and Amide-TBA. (18A) A schematic illustration of the mesophase transition from co-assembled mixed micelles into a hydrogel upon incubating with PGA enzyme. (18B) DLS spectrum of a co-assembled micellar formulation (1:1 DBA:TBA) at t=0 (solid line) and after 24 h incubation in PBS (pH 7.4) with PGA enzyme (dotted line) at 37° C. (18C) Overlay of HPLC chromatograms upon adding PGA. (18D) HPLC-based kinetic data. (18E) Images of the formulation at different time points.

FIGS. 19A-19D depict the mesophase transition from micelles to a hydrogel. (19A) Fluorescence spectra of micelles containing Amide-DBA and coumarin labeled Amide-TBA. (19B) Intensity at 480 nm, 540 nm and their ratio as a function of time after the addition of PGA at t=0. (19C) Absorbance spectra. (19D) Maximum intensity at 415 nm as a function of time.

FIGS. 20A-20B depict images of Amide-TBA-based hydrogel featuring its rheological properties.

FIG. 21 depicts amplitude sweep test of a hydrogel obtained from a mixture of Amide-DBA and Amide-TBA at a constant frequency of 1 Hz.

FIGS. 22A-22B depict chromatograms at 297 nm (22A) and 423 nm (22B) of a gel dissolved in acetonitrile following rheological measurements.

FIG. 23 depicts images of vials containing hydrogel with BSA or BSA and PGA over 13 days, indicating the transformation of the hydrogel into hydrolyzed polymers in the presence of PGA.

FIGS. 24A-24B depict chromatograms at 423 nm of the hydrolyzed top layer of the amphiphiles 13 days after exposure to BSA (24A) or BSA and PGA (24B).

FIGS. 25A-25E depict the mesophase transition from co-assembled mixed micelles into a hydrogel. (25A) Schematic illustration of the mesophase transition of Amide-DBA and Ester-TBA formulation from (i) co-assembled mixed micelles into (ii) a hydrogel upon incubating with PGA enzyme and no hydrogel formation in the presence of PLE enzyme. (25B) DLS spectra of a co-assembled micellar formulation (1:1 DBA:TBA) at t=0 (solid line) and after 24 h incubation in PBS (pH 7.4) with PGA enzyme (dotted line) at 37° C. (25C) Overlay of HPLC chromatograms upon addition to PGA at t=0 followed by addition of PLE at t=6 h. (25D) HPLC-based kinetic data. (25E) Images of the formulation at different time points.

FIGS. 26A-26D depict mixed micelles to hydrogel mesophase transition. (26A) Fluorescence spectra of micelles containing Amide-DBA and coumarin labeled Ester-TBA. (26B) Intensity at 480 nm, 540 nm and their ratio as a function of time after the addition of PGA at t=0 and PLE at t=6 h. (26C) Absorbance spectra. (26D) Maximum intensity at 415 nm as a function of time.

FIGS. 27A-27D depict mesophase transition from co-assembled mixed micelles into a hydrogel. (27A) DLS spectrum of a co-assembled micellar formulation (1:1 DBA:TBA) at t=0 (solid line) and after 24 h incubation in PBS (pH 7.4) with PLE enzyme (dotted line) at 37° C. (27B) Overlay of HPLC chromatograms upon addition of PLE at t=0 followed by addition of PGA at t=6 h. (27C) HPLC-based kinetic data. (27D) Images of the formulation at different time points.

FIGS. 28A-28B depict mixed micelles to hydrogel mesophase transition. (28A) Fluorescence spectra of micelles containing Amide-DBA and coumarin labeled Ester-TBA. (28B) Intensity at 480 nm, 540 nm and their ratio as a function of time following the addition of PLE at t=0 and PGA at t=6 h.

FIGS. 29A-29B depict chromatograms at 423 nm of a gel dissolved in acetonitrile following rheological measurements after exposure to PGA at t=0 and PLE at t=6 h (29A) or PLE at t=0 and PGA at t=6 h (29B).

FIGS. 30A-30B depict hydrogel characterization. (30A) Amplitude sweep test of the hydrogels obtained from Amide-DBA and Ester-TBA formulation at a constant frequency of 1 Hz. (30B) An image of Ester-TBA based hydrogel featuring its rheological properties.

FIGS. 31A-31B depict the mesophase transition from a hydrogel to soluble polymers by slow enzymatic degradation of the Ester-TBA. (31A) Schematic illustration of the slow enzymatic degradation of the Ester-TBA by PLE enzyme. (31B) Images of the vials containing hydrogel with BSA or BSA and PLE over 7 days, indicating the complete transformation of the hydrogel into hydrolyzed polymers in the presence of PLE.

FIGS. 32A-32B depict chromatograms at 423 nm of the hydrolyzed top layer of the amphiphiles 7 days after exposure to BSA (32A) or BSA and PGA (32B).

FIGS. 33A-33E depict the mesophase transition from co-assembled mixed micelles into a hydrogel. (33A) Schematic illustration of mesophase transition of Ester-DBA and Amide-TBA formulation from (i) co-assembled mixed micelles into (ii) a hydrogel upon incubating with PLE enzyme and no hydrogel formation in the presence of PGA enzyme. (33B) DLS spectra of a co-assembled micellar formulation (1:1 DBA:TBA) at t=0 (solid line) and after 24 h incubation in PBS (pH 7.4) with PLE enzyme (dotted line) at 37° C. (33C) Overlay of HPLC chromatograms upon addition of PLE at t=0 followed by addition of PGA at t=6 h. (33D) HPLC-based kinetic data. (33E) Images of the formulation at different time points.

FIGS. 34A-34D depict mixed micelles to hydrogel mesophase transition. (34A) Fluorescence spectra of micelles containing Ester-DBA and coumarin labeled Amide-TBA. (34B) Intensity at 480 nm, 540 nm and their ratio as a function of time following the addition of PLE at t=0 and PGA at t=6 h. (34C) Absorbance spectra. (34D) Maximum intensity at 415 nm as a function of time.

FIGS. 35A-35C depict mixed micelles to hydrogel mesophase transition. (35A) Overlay of HPLC chromatograms of Ester-DBA and Amide-TBA formulation following the addition of PGA at t=0 and PLE at t=6 h. (35B) Images of the formulation at different time points. (35C) HPLC-based kinetic data.

FIGS. 36A-36B depict the mesophase transition from co-assembled mixed micelles into a hydrogel. (36A) Fluorescence spectra of micelles containing Ester-DBA and coumarin labeled Amide-TBA. (36B) Intensity at 480 nm, 540 nm and their ratio as a function of time following the addition of PGA at t=0 and PLE at t=6 h.

FIGS. 37A-37B depict chromatograms at 423 nm of a gel dissolved in acetonitrile following rheological measurements after exposure to PLE at t=0 and PGA at t=6 h (37A) or PGA at t=0 and PLE at t=6 h (37B).

FIGS. 38A-38B depict chromatograms at 297 nm of a gel dissolved in acetonitrile following rheological measurements after exposure to PLE at t=0 and PGA at t=6 h (38A) or PGA at t=0 and PLE at t=6 h (38B).

FIG. 39 depicts the molecular structures of three DBA hybrids termed DBA-C6×2, DBA-C6×3 and DBA-C6×4 and a TBA hybrid according to embodiments of the present invention.

FIGS. 40A-40B depict the formation of a TBA-based hydrogel upon hydrolysis of DBA-C6×2 hybrid. (40A) Overlay of HPLC chromatograms upon addition of PLE at t=0. (40B) Images of TBA-based hydrogel formation at different time points.

FIG. 41 depicts an overlay of HPLC chromatograms of a formulation containing DBA-C6×3 and TBA upon addition of PLE at t=0.

FIG. 42 depicts an overlay of HPLC chromatograms of a formulation containing DBA-C6×4 and TBA upon addition of PLE at t=0.

FIG. 43 depicts the HPLC-based kinetic data of a formulation containing DBA-C6×2 and TBA upon addition of PLE at t=0.

FIG. 44 depicts the HPLC-based kinetic data of a formulation containing DBA-C6×3 and TBA upon addition of PLE at t=0.

FIG. 45 depicts the HPLC-based kinetic data of a formulation containing DBA-C6×4 and TBA upon addition of PLE at t=0.

FIG. 46A-46B depict hydrogel formation upon addition of PLE at t=0. (46A) Overlay of HPLC chromatograms of a formulation containing mPEG₅ₖ-(D)-4×C5 and TBA. (46B) HPLC-based kinetic data.

FIG. 47 depicts images of aging of the hydrogel over time.

FIG. 48 depicts an image of the hydrogel at t=24 h featuring hydrogel particles floating in the solution and settled hydrogel at the bottom of the vial.

FIGS. 49A-49B depict images of the hydrogel at different time points (49A) and at t=4 days (49B).

FIGS. 50A-50B depict images of the hydrogel at different time points (50A) Inverted vials. (50B) Upright vials.

FIGS. 51A-51B depict hydrogel formation upon addition of PLE at t=0. (51A) Overlay of HPLC chromatograms of a formulation containing mPEG₅ₖ-(D)-4×C6 and TBA. (51B) HPLC-based kinetic data.

FIGS. 52A-52B depict hydrogel formation upon addition of PLE at t=0. (52A) Overlay of HPLC chromatograms of a formulation containing mPEG₅ₖ-(D)-4×C7 and TBA. (52B) HPLC-based kinetic data.

FIG. 53 depicts enzymatic degradation of the DBA hybrids.

FIG. 54 depicts TBA-based hydrogel formation.

### DETAILED DESCRIPTION

The present invention provides delivery systems useful for releasing a cargo encapsulated within or attached thereto at a target site of interest. The delivery systems comprise amphiphilic polymers comprising a mixture of di-block copolymers and tri-block copolymers, each of the hydrophobic segments in the di- and tri-block copolymers comprise a stimulus responsive cleavable site. The delivery system of the present invention is configured to exhibit two sequential mesophase transitions in response to the stimuli thereby affording: 1) accumulation of a cargo at the target site following the first stimulus, and 2) sustained release of the cargo at the target site following the second stimulus.

According to the principles of the present invention the di-block copolymer is an amphiphilic surfactant comprising a hydrophilic segment and a hydrophobic segment, wherein the hydrophobic segment comprises a first stimulus-responsive cleavable site. The tri-block copolymer comprises two substantially identical hydrophobic segments each comprising a second stimulus-responsive cleavable site and a hydrophilic segment therebetween. The amphiphiles self-assemble or transform to micelles when immersed in an aqueous medium. Upon exposure to a first stimulus, cleavage of the first stimulus-responsive cleavable site occurs thereby disassembling the micelles and invoking a first phase transition to a hydrogel which undergoes subsequent degradation and a second phase transition to dissolved polymers or fragments thereof upon exposure to a second stimulus.

Di- and tri-block amphiphiles can form different mesophases ranging from micelles to hydrogels depending on their chemical structures, hydrophilic to hydrophobic ratios, and their ratio in a mixture. In addition, their architectures dictate their exchange rate between the assembled and unimer states, and consequently affect their responsiveness towards activating stimuli (e.g., enzymatic degradation). Disclosed therein for the first time is the utilization of the different reactivities of di- and tri-block amphiphiles towards different stimuli as a tool for programming formulations to undergo sequential stimuli-induced transitions from micelles to hydrogel and finally to dissolved polymers. The rate of the transition between the mesophases can be programmed by changing the ratio of the amphiphiles in the formulation. The assemblies disclosed herein can be utilized in a variety of formulations that can be tailored to adopt different mesophases in response to enzymatic stimuli.

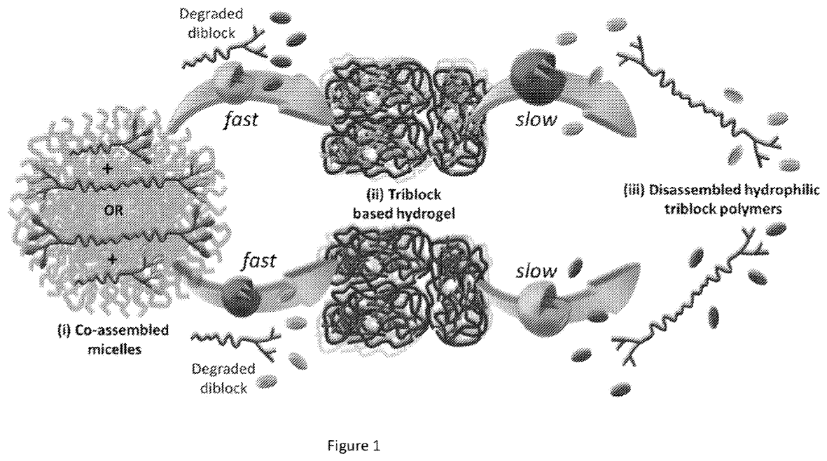

Reference is now made to FIG. 1 demonstrating certain features according to embodiments of the present invention. A delivery system that undergoes sequential multi-step mesophase transitions is provided herein. The delivery system incorporates amphiphiles of two different architectures, namely di-block amphiphiles (DBA) and tri-block (hydrophobic-hydrophilic-hydrophobic) amphiphiles (TBA) composed of dendrons as the hydrophobic side blocks. In some embodiments, the DBA and TBA feature identical dendron and the same hydrophilic to lipophilic balance (HLB) for example by using a PEG chain in the DBA having exactly half the molecular weight of the PEG chain in the TBA. A mixture of the DBA and TBA is co-assembled to micelles.

Activation of the amphiphiles using an enzyme capable of cleaving the hydrophobic end-groups is performed by incubating with an activating enzyme. A significantly higher selectivity of the enzyme towards degradation of the DBA is obtained. As the DBA can rapidly exchange between the micellar and unimer states, these hybrids are highly accessible towards an activating enzyme. In contrast, the TBA hybrids which are characterized by a higher molecular weight as well as a different architecture undergo a significantly slower exchange and hence are nearly unaffected by the degrading enzyme during the initial micellar state. Upon cleavage of the DBA beyond a certain threshold, these hybrids can no longer stabilize the micellar mesophase and the increase in the relative concentration of the TBA invokes the transition into a hydrogel mesophase. The hydrogel mesophase can be used as a polymeric-based depot for prolonged release of an encapsulated cargo at a target site for pharmaceutical, agricultural, cosmetic, and/or diagnostic utility.

According to some aspects and embodiments, there is provided a delivery system comprising self-assembled amphiphilic polymers in the form of micelles comprising a mixture of a di-block copolymer and a tri-block copolymer, wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween, and wherein the first stimulus-responsive cleavable site undergoes cleavage upon exposure to a first stimulus thereby invoking a first phase transition from micelles to a hydrogel, and the second stimulus-responsive cleavable sites undergo cleavage upon exposure to a second stimulus thereby disassembling the hydrogel and invoking a second phase transition to dissolved polymers and fragments thereof.

According to other aspects and embodiments, there is provided a delivery system comprising a mixture of a di-block copolymer and a tri-block copolymer, wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween, wherein the delivery system is in the form of fibers which are transformed to micelles upon hydration, and wherein the first stimulus-responsive cleavable site undergoes cleavage upon exposure to a first stimulus thereby invoking a first phase transition from micelles to a hydrogel, and the second stimulus-responsive cleavable sites undergo cleavage upon exposure to a second stimulus thereby disassembling the hydrogel and invoking a second phase transition to dissolved polymers and fragments thereof.

Thus, provided herein is a delivery system which may be in the form of micelles or in the form of fibers capable of self-assembling/transforming into micelles when hydrated. The terms “micelles” and/or “micellar form” used herein interchangeably refer to nanosized spherical, flower-like, ellipsoid, cylindrical, or unilamellar structures that are formed by self-assembly of components having hydrophobic and hydrophilic segments. The micelles typically have an average particle size of less than about 100 nm, preferably about 50 nm or lower, more preferably about 5 nm to 50 nm, and most preferably about 5 nm to 20 nm, including each value within the specified ranges. The term “fibers” as used herein refers to structures characterized by having one of the dimensions (referred to as the length of the fiber) elongated with respect to the other dimension. It is to be understood that the term “fibers” as used herein refers to structures in the nanometer as well as micrometer range. Typically, the fibers have lengths ranging from about 100 μm to a few millimeters and widths ranging from about 100 nm to about 100 μm, including each value within the specified ranges. The term “self-assembly” as used herein refers to a process in which the amphiphiles form an organized structure due to specific interactions including, but not limited to, van der Waals forces, hydrophobic interactions, hydrogen bonds, and the like, without external direction or trigger upon exposure to a medium, for example an aqueous medium. Although external factors might affect self-assembly, it is contemplated that this process occurs mainly due to the structure of the amphiphiles.

According to the principles of the present invention, the di-block copolymers are characterized by featuring a hydrophilic segment and a hydrophobic segment, wherein the hydrophobic segment comprises a first stimulus-responsive cleavable site, and the tri-block copolymer comprises two substantially identical hydrophobic segments each comprising a second stimulus-responsive cleavable site and a hydrophilic segment therebetween. Thus, the tri-block copolymers are characterized by featuring the following structure: B-A-B, where B is a hydrophobic segment comprising the second stimulus-responsive cleavable site and A is a hydrophilic segment.

Encompassed by the present invention are delivery systems comprising a mixture of a di-block copolymer and a tri-block copolymer at a mole ratio of about 1:10 to about 10:1, including all iterations of ratios within the specified range. For example, the delivery system may comprise a mixture of a di-block copolymer and a tri-block copolymer at mole ratios of from about 1:5 to about 5:1, or from about 1:3 to about 3:1, including all iterations of ratios within the specified ranges. Exemplary molar ratios of the di- to tri-block copolymers include, but are not limited to, about 1:10, about 1:9, about 1:8, about 1:7, about 1:6, about 1:5, about 1:4, about 1:3, about 1:2, about 1:1, about 2:1, about 3:1, about, 4:1, about 5:1, about 6:1, about 7:1, about 8:1, about 9:1, or about 10:1. Each possibility represents a separate embodiment. Currently preferred molar ratios of the di- to tri-block copolymers include, but are not limited to, about 1:1, about 2:1, and about 3:2. Each possibility represents a separate embodiment.

### 1. Hydrophilic Segment:

In some aspects and embodiments, the hydrophilic segments in the di- and/or tri-block copolymers comprise a hydrophilic polymer which may be the same or different with each possibility representing a separate embodiment. Suitable hydrophilic polymers include, but are not limited to, polyacrylic acid, poly(hydroxyethyl acrylate), polyethylene glycol (PEG), poly(oligo-ethylene glycol acrylate), polyacrylamide, polymethyl oxazoline, polyethyl oxazoline, polysarcosine, polypeptide, polypeptoid, hydrophilic polymethacrylate, polyamine, hydrophilic nylon, polyvinyl alcohol, hydrophilic protein, and polycarbohydrate. Each possibility represents a separate embodiment. In particular aspects and embodiments, the hydrophilic polymers include, but are not limited to, polyacrylic acid, poly(2-hydroxyethyl acrylate), polyethylene glycol (PEG), and poly(oligo-ethylene glycol acrylate). Each possibility represents a separate embodiment. A currently preferred hydrophilic polymer comprises polyethylene glycol (PEG), for example PEG having at least 10 repeating units of ethylene glycol monomers.

Typically, the hydrophilic segment has a molecular weight of about 0.5 to about 100 kDa, including each value within the specified range. Exemplary molecular weights of the hydrophilic segments within the scope of the present invention include, but are not limited to, from about 0.5 to about 75 kDa, from about 0.5 to about 50 kDa, or from about 0.5 to about 25 kDa, including each value within the specified ranges. In one embodiment, the molecular weight of the hydrophilic segment of the di-block copolymer is from about 0.5 to about 10 kDa, including each value within the specified range. In another embodiment, the molecular weight of the hydrophilic segment of the tri-block copolymer is from about 5 to about 15 kDa, including each value within the specified range. In one embodiment, the hydrophilic segment in the di-block copolymer has half the molecular weight of the hydrophilic segment in the tri-block copolymer. In some embodiments, the hydrophilic segment in the di- and/or tri-block copolymer does not contain a peptide or a polypeptide.

The hydrophilic segment is chemically bound in at least one termini to a hydrophobic segment to form the di- and/or tri-block copolymers of the present invention. Typical groups in said chemical bonds include, but are not limited to, —Z—X—, —X¹—Z—X²—, and —Z¹—X¹—Z²—X²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof. Each possibility represents a separate embodiment.

For the sake of clarity, the optional groups that chemically bond between the hydrophilic and hydrophobic segments are depicted in one direction. However, it is to be understood by the person having ordinary skill in the art that the relevant embodiments are not limited to a specific direction, for example, the group —X¹—Z—X²—, in a di-block copolymer may refer to either (hydrophobic segment)-X¹—Z—X²-(hydrophilic segment) or to (hydrophobic segment)-X²—Z—X¹-(hydrophilic segment). Furthermore, groups that are described with direction are intended to cover both directions, e.g., —C(═O)—NH-refer also to —NH—C(═O)—.

Currently preferred linkages between the hydrophilic segment(s) and the hydrophobic segment(s) of the di- and/or tri-block copolymers are —X¹—Z—X²—, wherein Z is C₂ alkenylene and X¹ and X² are each —C(═O)—NH—. In some embodiments, the linkages between the hydrophilic segment and the two hydrophobic segments of the tri-block copolymer are the same.

### 2. Hydrophobic Segments:

In some aspects and embodiments, the hydrophobic segments in the di- and/or tri-block copolymers comprise a hydrophobic dendron, which may be the same or different with each possibility representing a separate embodiment. A “dendron” as used herein is a hyper-branched monodisperse organic molecule defined by a tree-like or generational structure. In general, dendrons possess three distinguishing architectural features: a linker moiety; an interior area containing generations with radial connectivity to the linker moiety; and a surface region (peripheral region) of terminal moieties. According to certain embodiments, each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof. Each possibility represents a separate embodiment.

In certain aspects and embodiments, each generation is derived from a compound having a structure represented by the formulae HX—Z—XH or HX—Z—CO₂H, wherein X is independently at each occurrence NH, S or O, and Z is selected from C₁-C₁₀ alkylene, C₂-C ₁₀ alkenylene, C₂-C₁₀ alkynylene, and arylene. Each possibility represents a separate embodiment. According to other embodiments, each generation of the dendron is derived from a compound selected from the group consisting of HX—CH₂—CH₂—XH, HX—(CH₂)₁₋₃—CO₂H, and HX—CH₂—CH(XH)—CH₂—XH wherein X is independently at each occurrence NH, S or O. Each possibility represents a separate embodiment. In one currently preferred embodiment, each generation of the dendron is derived from a compound selected from the group consisting of HS—CH₂—CH₂—OH, HS—(CH₂)₁₋₃—CO₂H and HS—CH₂—CH(OH)—CH₂—OH. Each possibility represents a separate embodiment.

The hydrophobic dendron of the present invention comprises a preferred number of generations in the range of 0 to 5, more preferably 0 to 3, including each integer within the specified ranges. In one embodiment, the hydrophobic dendron is a generation 0 (G0) dendron. In another embodiment, the hydrophobic dendron is a generation 1 (G1) dendron. In yet another embodiment, the hydrophobic dendron is a generation 2 (G2) dendron. In other embodiments, the hydrophobic dendron is a generation 3 (G3) dendron.

In various embodiments, the dendron comprises a repeating unit selected from the group consisting of:

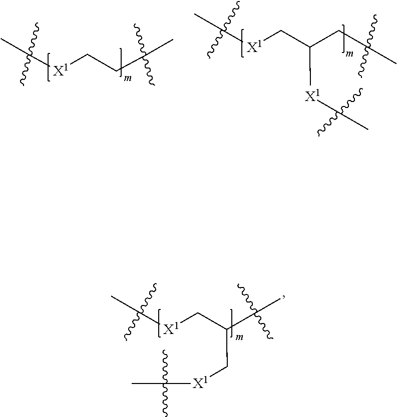

wherein X¹ is independently, at each occurrence, selected from the group consisting of O, S and NH; and m is an integer from 1 to 15, including each integer within the specified range.

According to the principles of the present invention, the hydrophobic segment comprises at least one stimulus-responsive cleavable site, for example an enzymatically cleavable site. Enzymatically cleavable sites typically include a functional group such as, but not limited to, an ester, an amide, a carbamate, a carbonate, a urea, a sulfate, an amidine, an ether, a phosphate, a phosphoamide, a sulfamate, a nitro, an azo, and a trithionate. Each possibility represents a separate embodiment. Functional groups that can be cleaved by enzymes include, for example —O—C(O)—R′, —C(O)—OR′—NH—C(O)—R′ or —C(O)—NHR′ wherein R′ is C₁-C₁₂ alkyl or an aryl. Each possibility represents a separate embodiment.

It will be appreciated to one skilled in the art that an amide bond is enzymatically cleavable by an amidase. Suitable amidases that can cleave an amide bond include, but are not limited to, aryl-acylamidase, aminoacylase, alkylamidase, and phthalyl amidase. Each possibility represents a separate embodiment. Where an ester bond is present in the hydrophobic segment, it can be cleaved by an esterase. Suitable esterases that can cleave an ester bond include, but are not limited to, carboxylesterase, arylesterase, and acetylesterase.

Each possibility represents a separate embodiment. Where a urea bond is present in the hydrophobic segment, it can be cleaved by a urease.

Where hydrophobic segments comprise hydrophobic dendrons, the enzymatically cleavable site may be present at one or more of the terminal repeating units (i.e., terminal generations) of the hydrophobic dendron, and/or in intermediary generations of the dendron.

The enzymatically cleavable hydrophobic end group may be present only at the terminal repeating units of the hydrophobic dendron (i.e., the enzymatically cleavable hydrophobic end group is not present in intermediary generations of the dendron) or it may be present only at the intermediary generations of the dendron (i.e., the enzymatically cleavable hydrophobic end group is not present in the terminal repeating units of the hydrophobic dendron). Each possibility represents a separate embodiment.

The term “alkyl” used herein alone or as part of another group denotes a saturated aliphatic hydrocarbon, including straight-chain and branched-chain alkyl groups. In one embodiment, the alkyl group has 1-12 carbons designated here as C₁-C₁₂ alkyl. In another embodiment, the alkyl group has 1-4 carbons designated here as C₁-C₄ alkyl. Examples of alkyl groups include, but are not limited to, methyl, ethyl, propyl, isopropyl, n-butyl, isobutyl, sec-butyl, t-butyl, and the like.

The term “alkylene” used herein alone or as part of another group denotes a bivalent radical which is bonded at two positions connecting together two separate additional groups (e.g., CH₂). Examples of alkylene groups include, but are not limited to —(CH₂)—, —(CH₂)₂—, —(CH₂)₃—, —(CH₂)₄—, etc.

The term “alkenylene” used herein alone or as part of another group denotes a bivalent radical containing at least one double bond, which is bonded at two positions connecting together two separate additional groups (e.g., —CH═CH—).

The term “alkynylene” used herein alone or as part of another group denotes a bivalent radical containing at least one triple bond, which is bonded at two positions connecting together two separate additional groups (e.g., —C≡C—).

The term “aryl” used herein alone or as part of another groups denotes an aromatic ring system containing from 5-14 ring carbon atoms. The aryl ring can be a monocyclic, bicyclic, tricyclic and the like. Non-limiting examples of aryl groups are phenyl, naphthyl including 1-naphthyl and 2-naphthyl, and the like.

The term “arylene” denotes a bivalent radical of aryl, which is bonded at two positions connecting together two separate additional groups (e.g., —C₆H₄—).

Each of the alkyl, alkylene, alkenylene, alkynylene, aryl, and arylene can be substituted by one or more of the following substituents methyl, ethyl, propyl, isopropyl, butyl, tert-butyl, halogen, haloalkyl, hydroxy, alkoxy, carbonyl, amido, alkylamido, dialkylamido, nitro, cyano, amino, alkylamino, dialkylamino, carboxyl, thio, and thioalkyl. Each possibility represents a separate embodiment.

All stereoisomers, optical and geometrical isomers of the compounds of the instant invention are contemplated, either in admixture or in pure or substantially pure form. For example, when the compounds of the present invention have asymmetric centers, they can exist in enantiomeric or diastereomeric forms or in mixtures thereof. The present invention contemplates the use of any racemates (i.e., mixtures containing equal amounts of each enantiomers), enantiomerically enriched mixtures (i.e., mixtures enriched in one enantiomer), pure enantiomers or diastereomers, or any mixtures thereof. The chiral centers can be designated as R or S or R,S or d,D, l,L or d,l, D,L. In addition, several of the compounds of the invention contain one or more double bonds. The present invention intends to encompass all structural and geometrical isomers including cis, trans, E and Z isomers, independently at each occurrence. Any salt form with both basic and acid addition salts is also contemplated within the scope of the present invention.

In some aspects and embodiments, the di- and tri-block copolymers are synthetic. The di- and/or tri-block copolymers of the present invention can be synthesized by methods well known to those skilled in the art, for example using an atom transfer radical polymerization, a click reaction, and the like.

In some aspects and embodiments, the hydrophilic to lipophilic balance (HLB) of the di-block copolymer is substantially identical to the HLB of the tri-block copolymer. As used herein, the term “hydrophilic to hydrophobic ratio” refers to the ratio of the hydrophilic units to the hydrophobic units. In other embodiments, the term “hydrophilic to hydrophobic ratio” refers to the hydrophilic to lipophilic balance (HLB) which represents the degree of affinity to water and oil. Specifically, wherein the HLB is close to zero, the amphiphile is considered highly hydrophobic, and wherein the HLB is close 20, the amphiphile is considered highly hydrophilic. The HLB value may be determined by any method known in the art, for example the Atlas method, the Griffin method, the Davis method, or the Kawakami method. Each possibility represents a separate embodiment. As used herein, the term “substantially” refers to a deviation that is of not more than ±10% of the hydrophilic to hydrophobic ratio or the HLB value.

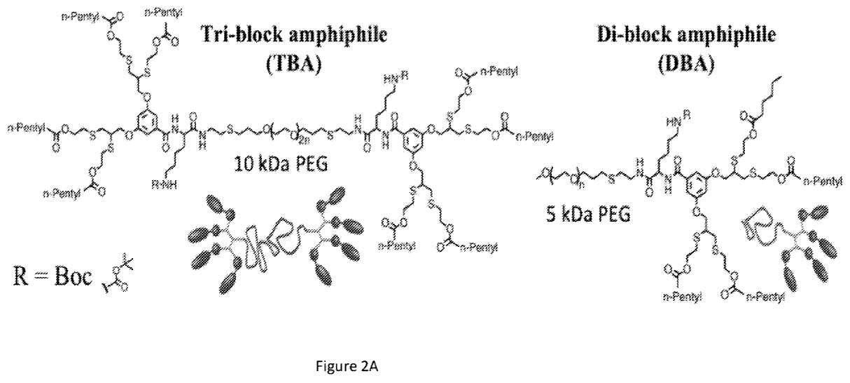

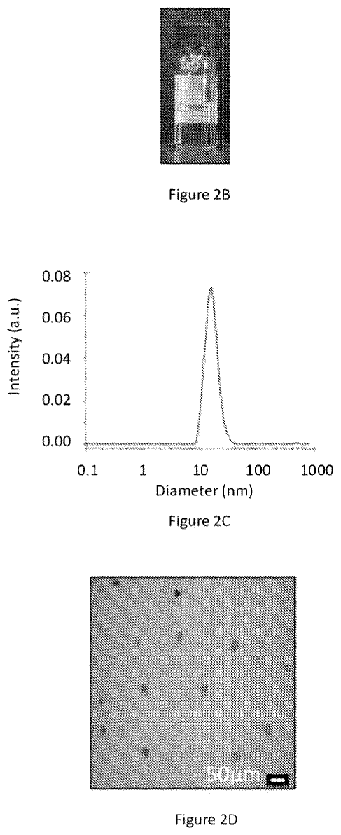

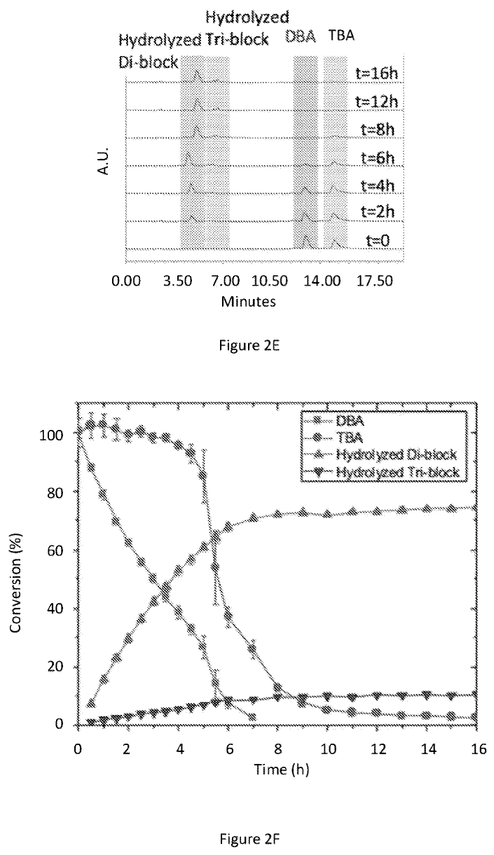

Currently preferred di- and tri-block copolymers in accordance with the principles of the present invention are represented by the structures depicted in any one of FIGS. 2A, 17, and 39. Each possibility represents a separate embodiment.

### 3. Mesophase Transitions:

According to the principles of the present invention, the delivery system of the present invention is designed to undergo at least two mesophase transitions. The first mesophase transition is induced by the cleavage of the first stimulus-responsive cleavable site and it involves the transition from micelles into a hydrogel. The second phase transition is induced by the cleavage of the second stimulus-responsive cleavable site and it involves the transition from a hydrogel to dissolved polymers and fragments thereof.

The cleavages of the first and second cleavable sites are performed by the application of a first and second stimuli, respectively, which may be the same or different with each possibility representing a separate embodiment. The first and second stimuli may be, independently, chemically-induced or physically-induced with each possibility representing a separate embodiment. Physically induced stimuli include, but are not limited to, a change in at least one of temperature, pH, light (UV light, visible light or near infrared light), or electric field. Each possibility represents a separate embodiment. Chemically induced stimuli include, but are not limited to, contacting the amphiphiles with an agent such as a redox agent, a transport protein or an activating enzyme. Each possibility represents a separate embodiment. As used herein, the term “contacting” refers to bringing in contact with the di- and/or tri-block copolymers of the present invention. Contacting can be accomplished for example in cells or tissue cultures, or living organisms. Each possibility represents a separate embodiment. In one embodiment, the present invention encompasses contacting the delivery system of the present invention with an agent within a human subject. In other embodiments, the term “contacting” may be performed ex-vivo on a surface, on a device, in cell/tissue culture dish, in food and water. Each possibility represents a separate embodiment.

In currently preferred embodiments, the agent that induces cleavages of the first and second cleavable sites is an activating enzyme. Suitable activating enzymes within the scope of the present invention include, but are not limited to, amidases, esterases, and ureases, as detailed hereinabove.

While different agents may be used to induce the first and second cleavages according to the principles of the present invention, it is to be understood that the same agent may be used to induce both. Even though the same agent may be used to induce both cleavages, according to the principles of the present invention, the first cleavage occurs at a higher rate than the second cleavage. Thus, in some embodiments, the rate of the first cleavage is at least 1.2-fold higher than the rate of the second cleavage. In other embodiments, the rate of the first cleavage is at least 1.5-fold higher than the rate of the second cleavage. In yet other embodiments, the rate of the first cleavage is at least 1.7-fold higher than the rate of the second cleavage. In further embodiments, the rate of the first cleavage is at least 2-fold higher than the rate of the second cleavage.

In some aspects and embodiments, the first cleavage induces a phase transition from micelles to a hydrogel. The term “hydrogel” as used herein refers to a three-dimensional hydrated assembly of the amphiphiles having characteristic viscoelastic properties. Typically, the water content of the hydrogels is at least about 10% to about 20%, about 30% to about 40%, about 50% to about 70%, about 75% to about 95%, or about 80% to about 99%, including each value within the specified ranges.

In some aspects and embodiments, the second cleavage induces a phase transition from a hydrogel to dissolved or degraded polymers or fragments thereof. Advantageously, the di- and tri-block copolymers of the present invention are biodegradable such that following the second cleavage, degradation of the amphiphiles occur. The term “biodegradable” as used herein refers to a component which erodes or degrades at its surfaces over time due, at least in part, to contact with substances found in the surrounding tissue fluids, or by cellular action.

### 4. Cargo Release and Uses:

In some aspects and embodiments, the delivery system disclosed herein comprises a cargo encapsulated within the micelles. In other aspects and embodiments, the delivery system disclosed herein comprises a cargo covalently attached to the micelles. The attachment, according to the principles of the present invention, comprises a covalent bond between the cargo and the hydrophobic segment of the di-block copolymer and/or the hydrophobic segments of the tri-block copolymer. Each possibility represents a separate embodiment.

Suitable cargo includes, but is not limited to, pharmaceutical agents, agrochemical agents, cosmetic agents, imaging agents, and/or diagnostic agents. Each possibility represents a separate embodiment.

Pharmaceutical agents include, but are not limited to, drugs which may be small molecules or biologics. Non-limiting examples of drugs include chemotherapeutic agents, anti-proliferative agents, anti-cancer agents, inhibitors, receptor agonists, receptor antagonists, co-factors, anti-inflammatory drugs (steroidal and non-steroidal), antipsychotic agents, analgesics, anti-thrombogenic agents, anti-platelet agents, anticoagulants, anti-diabetics, statins, toxins, antimicrobial agents, anti-histamines, metabolites, anti-metabolic agents, vasoactive agents, vasodilator agents, cardiovascular agents, antioxidants, phospholipids, and heparins. Each possibility represents a separate embodiment.

Pharmaceutical agents also include peptides, polypeptides, hormones, polymers, amino acids, oligonucleotides, nucleic acids, genes, growth factors, enzymes, co-factors, antisense molecules, antibodies, antigens, vitamins, immunoglobulins, cytokines, prostaglandins, vitamins, toxins and the like, as well as organisms such as bacteria, viruses, fungi and the like. Each possibility represents a separate embodiment.

Agrochemical agents include, but are not limited to, a pesticide, an insecticide, a herbicide, a fungicide, an acaricide, an algicide, an antimicrobial agent, biopesticide, a biocide, a disinfectant, a fumigant, an insect growth regulator, a plant growth regulator, a miticide, a microbial pesticide, a molluscide, a nematicide, an ovicide, a pheromone, a repellent, a rodenticide, a defoliant, a dessicant, a termiticide, a piscicide, avicide, rodenticide, bactericide, insect repellent, an auxin, a cytokinin, a gametocide, a gibberellin, a growth inhibitor, a growth stimulator and any combination thereof. Each possibility represents a separate embodiment.

Cosmetic agents include, but are not limited to, hyaluronic acid, vitamins and vitamin derivatives such as, for example vitamin A, vitamin B, vitamin D, vitamin E, vitamin K and derivatives thereof including, for example, a tocopherol; various plant extracts such as, for example aloe vera, aloe barbadensis, castor oil, Citrus limonium, Citrus paeadisi, Citrus sinensis, Elaesis guineensis, etc. sunscreens and tanning agents and the like. Each possibility represents a separate embodiment.

Imaging and/or diagnostic agents include, but are not limited to, labeling compounds or moieties such as chromophores, fluorescent compounds or moieties, phosphorescent compounds or moieties, contrast agents, radioactive agents, magnetic compounds or moieties (e.g., diamagnetic, paramagnetic and ferromagnetic materials), heavy metal clusters and the like. Each possibility represents a separate embodiment.

The cargo is typically present in the delivery system in amounts sufficient so as to exert its beneficial effect once released therefrom.

According to the principles of the present invention, when the cargo is attached to the tri-block copolymer, cargo delivery mainly occurs following the second phase transition. In particular, it is contemplated that the first phase transition in which the micelles are disassembled and a tri-block copolymer-based hydrogel is formed does not afford the release of the cargo that is attached to the tri-block copolymer. Only following the second phase transition in which disassembly of the hydrogel occurs, the cargo is released from the delivery system in a slow- or sustained-release manner.

The terms “slow” or “sustained” as used herein refer to a delivery system or formulation that provides prolonged, long or extended release of a cargo at the target site. This term may further refer to a delivery system or formulation that provides prolonged, long or extended exposure to and duration of action of the cargo at the target site. According to various aspects and embodiments, the release of the cargo occurs in a continuous manner. The release profile can be a zero order release profile, a first order release profile, a second order release profile, a third order release profile, or any pseudo orders known. Each possibility represents a separate embodiment. Without being bound by a particular theory it is believed that the release of the cargo can occur by either one of two different mechanisms.

The first mechanism includes the release by diffusion through the polymer matrix. The second mechanism includes the release due to degradation of the polymer, for example by its hydrolysis.

The delivery system of the present invention can further be provided in the form of a kit whereby a first compartment comprises a mixture of a di-block copolymer and a tri-block copolymer, a second component comprises an agent (e.g., a first enzyme) capable of cleaving the first stimulus-responsive cleavable site, and a third component comprises an agent (e.g., a second enzyme) capable of cleaving the second stimulus-responsive cleavable site. In other embodiments, the delivery system of the present invention is provided in the form of a two-component kit whereby a first compartment comprises a mixture of a di-block copolymer and a tri-block copolymer, a second component comprises an agent (e.g., an activating enzyme) capable of cleaving the first and second stimulus-responsive cleavable sites. Typically, each of the components may further comprise a suspending agent, a buffer, and the like. For example, when the enzyme is in a lyophilized form, the kit may optionally contain a sterile and physiologically acceptable reconstitution medium such as water, saline, buffered saline, and the like. Each possibility represents a separate embodiment. According to some embodiments, associated with such compartments in the kit may be various written instructions for use.

Where a range of values is provided, it is understood that each intermediate value or intermediate range are also encompassed within the invention. It is further understood that the upper and lower limits of a range of values are also included in the invention.

The term “about” as used herein refers to ±10% of a specified value.

Throughout the description and claims, the words “comprise” and “contain” and variations of the words, for example “comprising” and “comprises”, mean “including but not limited to”, and are not intended to (and do not) exclude other moieties, additives, components, integers or steps.

As used herein, the singular forms “a”, “an”, and “the” include plural forms unless the context clearly dictates otherwise. Thus, for example, reference to “a cleavable site” also includes a plurality of cleavable sites, which may be the same or different with each possibility representing a separate embodiment.

As used herein, the term “and” or the term “or” include “and/or” unless the context clearly dictates otherwise.

Various embodiments and aspects of the present invention as delineated hereinabove and as claimed in the claims section below find experimental support in the following examples.

### EXAMPLES

### Instrumentation:

HPLC: All measurements were recorded on a Waters Alliance e2695 separations module equipped with a Waters 2998 photodiode array detector. All solvents (HPLC grade) were purchased from Bio-Lab Chemicals and were used as received.

¹H and ¹³C-NMR: spectra were recorded on Bruker Avance III 400 MHz/100 MHz spectrometer. The molecular weights of the dendron-PEG-dendron tri-block copolymers were determined by comparison of the areas of the peaks corresponding to the PEG block (3.63 ppm) and the protons peaks of the dendrons.

GPC: All measurements were recorded on Viscotek GPC max by Malvern using refractive index detector and PEG standards (purchased from Sigma-Aldrich) were used for calibration.

DLS: All measurements were recorded on a Corduan Technology VASCOy particle size analyzer.

Fluorescence Spectra: All spectra were recorded on an Agilent Technologies Cary Eclipse Fluorescence Spectrometer using quartz cuvettes.

Confocal microscopy: All images were taken in Olympus IX83 FLUOVIEW™ FV3000 confocal microscope.

Rheological measurements: A controlled-stress rheometer (AR-G2, TA instruments, USA) was used with an 8 mm diameter flat-plate geometry and a crosshatched surface. The viscoelastic region was determined by an amplitude sweep spanning from 0.01 to 100% at a frequency of 1 Hz and a temperature of 25° C. while maintaining a gap size of 0.9 mm.

### Materials:

Poly(ethylene glycol) (10 kDa), poly(ethylene glycol) methyl ether (5 kDa), allyl bromide (99%), 2,2-dimethoxy-2-phenylacetophenone (DMPA, 99%), Fmoc-Lys(Boc)-OH Novabiochem®, N,N′-Diisopropylcarbodiimide, propargyl bromide, 80% solution in toluene, 4-nitrophenol (99.5%), 4-dimethylamino pyridine (DMAP), N,N′-dicyclohexylcarbodiimide (DCC, 99%), 2-mercaptoethanol, Indolium, 2,3,3-trimenthylindolenine, hexanoic acid, 6-bromohexanoic acid, acetic acid, aniline, triethyl formate, triethylsilane, and Sephadex® LH20 were purchased from Sigma-Aldrich. 3,5 dihydroxy benzoic acid, N-(3-(phenylimino)propenyl)aniline, and 1,3-bis(prop-2-yl)carbodiimide (DIC, 99%) were purchased from Apollo Scientific Ltd. Anhydrous potassium carbonate, sodium acetate and trifluoroacetic acid (TFA) were purchased from Alfa Aesar. Potassium hydroxide, cystamine hydrochloride, Oxyma Pure Novabiochem® triphenylmethyl chloride, acetic anhydride, diisopropylethylamine (DIPEA), porcine liver esterase (PLE), and bovine serum albumin (BSA, Probumin®) were purchased from Merck. 2-(1H-benzotriazole-1-yl)-1,1,3,3-tetramethyluronium hexafluorophosphate was purchased from Chem-Impex. Piperidine, Silica Gel 60 Å 0.040-0.063 mm, sodium hydroxide, and all solvents were purchased from Bio-Lab and were used as received. Deuterated solvents for NMR were purchased from Cambridge Isotope Laboratories (CIL).

### Example 1

### Synthesis

DBA and TBA having structures depicted in FIG. 2A were synthesized as follows:

### Preparation of Di-Block Amphiphiles (DBA):

mPEG-Lys(Boc)-[dend-(yne)₂](1) was synthesized as reported in Buzhor et al, (Chem. Eur. J. 2015, 21, 15633-15638), the contents of which are hereby incorporated herein in their entirety. Compound (2) was synthesized as reported in Wulf et al., (ACS Nano. 2021, 15, 20539-20549), the contents of which are hereby incorporated herein in their entirety. The synthesis route for the preparation of mPEG₅ₖ-Lys(Cy5)-[dend-(hexanoate)₄] is shown in Scheme 1.

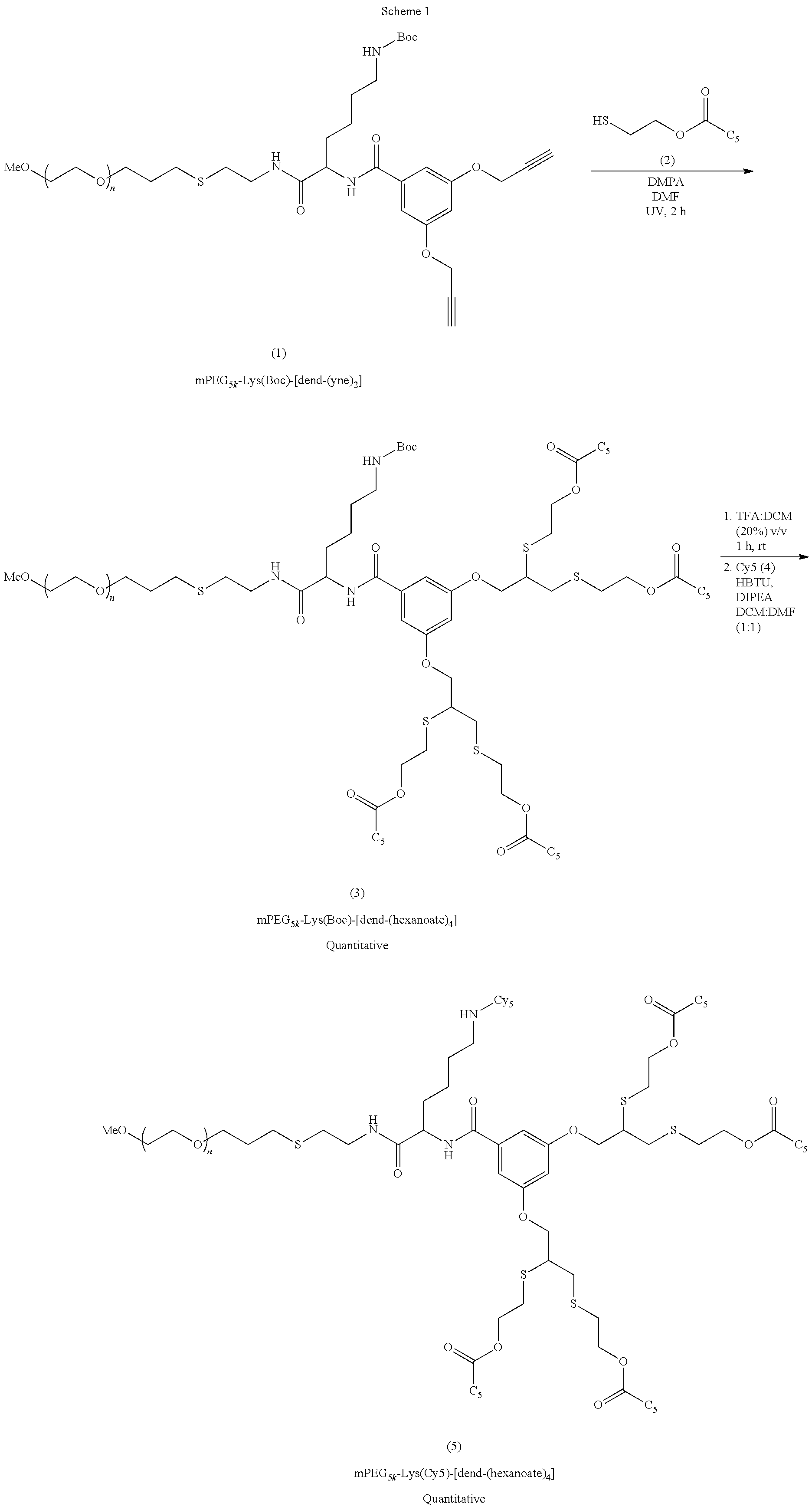

200 mg of compound (1) (0.036 mmol) were dissolved in DMF (0.5 mL). Compound (2) (254 mg, 1.44 mmol) and DMPA (3.7 mg, 0.014 mmol) were added to the solution. The solution was purged with nitrogen for 15 minutes and then placed under UV light at 365 nm for 2 hours. The crude mixture was loaded on a MeOH-based LH20 SEC column. The fractions that contained the product were unified, and MeOH was evaporated in vacuum. Further purification was performed by re-dissolving the oily residue in DCM (1 mL) followed by precipitation by the dropwise addition of ether (50 mL). The white precipitate was filtered, washed twice with ether, and dried under a high vacuum. The product, mPEG-Lys(Boc)-[dend-(hexanoate)₄](3), was obtained as a white solid (quantitative yield).

¹H-NMR (CDCl₃): δ 7.02 (d, J:==7.5 Hz, 1H, Ar—H), 6.98 (d, J=2.8 Hz, 2H, Ar—H), 6.68 (m, 1H, —CH₂—NH—CO—CH—). 6.60 (d, J 2.6 Hz, 1H, —CH—NH—CO—C—), 4.68 (m, 1H, —NH-Boc), 4.54 (q, J=7.3 Hz, 1H, —CO—CH—NH—), 4.33-4.12 (m, 12H, —Ar—O—CH₂—+CH₂—O—CO—C₅), 3.83-3.42 (m, PEG backbone), 3.36 (s, 3H, CH₃—O-PEG), 3.20 (q, J=5.5 Hz, 2H, —CH—S—), 3.14-2.48 (m, 18H, —S—CH₂—CH₂—NH—+—CH₂—NH-Boc+—CH—CH₂—S—+—CH—CH ₂—S—CH₂+CH₂—CH₂—S—), 2.28 (t, J=7.5 Hz, 8H, —O—CO—CH₂), 2.04-1.66 (n, 4 Hz, —O—CH₂—CH₂—CH₂—S—+Boc-NH—(CH₂)₃CH₂—CH—), 1.54-1.38 (m, 13H, Boc-NH—CH₂—CH₂—CH₂—CH₂—+Boc), 1.35-1.15 (m, 24H, —O—CO—CH₂—CH₂+—O—CO—(CH₂)₂—CH₂+—O—CO—(C-2)₃—CH₂), 0.86 (t, J=6.3 Hz 121, —O—CO—(CH₂)₄—CH₃). ¹³C-NMR (CDCl₃) δ 173.6, 171.4, 166.7, 159.4, 156.0, 136.0, 106.1, 104.6, 70.43, 63.31, 63.0, 59.0, 53.4, 45.4, 40.0, 38.5, 34.7, 34.1, 32.0, 31.4, 30.3, 29.5, 28.3, 28.1, 24.5, 22.7, 22.2, 13.8. GPC (DMF+25 mM NH₄Ac): calculated Mn=6.3 kDa, experimental Mn=6.3 kDa, DM=1.08.

Cy5 fluorescent dye (4) was synthesized as reported in Jung et al., (Bioorganic and Medicinal Chemistry 2006, 15, 92-97), the contents of which are hereby incorporated herein in their entirety.

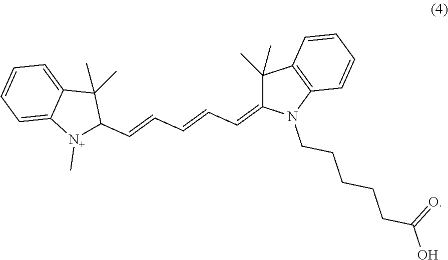 [SMILES: C[NH+]1c2ccccc2C(C)(C)C1/C=C/C=C/C=C1/N(CCCCCC(=O)O)c2ccccc2C1(C)C]

100 mg (0.016 mmol) of compound (3) was dissolved in DCM (1 mL) and TFA (200 μL). The mixture was allowed to stir for 1 h and the reaction was monitored via HPLC. Once the Boc deprotection was confirmed by HPLC, DIPEA was added to the reaction mixture until fume release was terminated. Then, MeOH-based LH20 SEC column was performed to discard the TFA.

Cy5 (29.3 mg, 0.048 mmol), HBTU (18.2 mg, 0.048 mmol) and DIPEA (31 mg, 0.239 mmol) were dissolved in a total volume of 1 mL DCM:DMF (1:1 v/v) in a 4 mL vial. The solution was stirred for 2 minutes and then added to the concentrated polymer hybrid purified by MeOH-based 11-120 SEC column. The reaction mixture was allowed to stir for 2 h and then loaded on MeOH-based LH20 SEC column. The fractions that contained the product, mPEG-Lys(Cy5)-[dend-(hexanoate)₄](5), were unified, MeOH was evaporated in vacuum, and the product was further dried under a high vacuum. The product (5) was obtained as a dark blue solid (quantitative yield).

¹³C-NMR (CDCl₃): 173.5, 166.4, 159.3, 153.1, 152.1, 142.7, 140.9, 136.5, 128.8, 128.6, 126.2, 125.4, 124.8, 122.0, 111.1, 110.1, 106.4, 105.1, 104.7, 103.3, 70.4, 63.3, 63.0, 58.9, 54.5, 49.3, 45.5, 44.4, 39.2, 37.7, 35.9, 34.8, 34.0, 31.5, 31.2, 30.9, 30.2, 29.5, 28.3, 28.0, 26.9, 26.0, 25.0, 24.5, 22.7, 22.2, 13.8.

### Preparation of Tri-Block Amphiphiles (TBA)

bPEG₁₀ₖ-Amine (6) was synthesized as reported in Edelstein-Pardo et al., (Chem. Mater. 2022, 34, 6367-6377), the contents of which are hereby incorporated herein in their entirety. The synthesis route for the preparation of bPEG₁₀ₖ-Lys(Boc)-[dend-(yne)₂] is shown in Scheme 2.

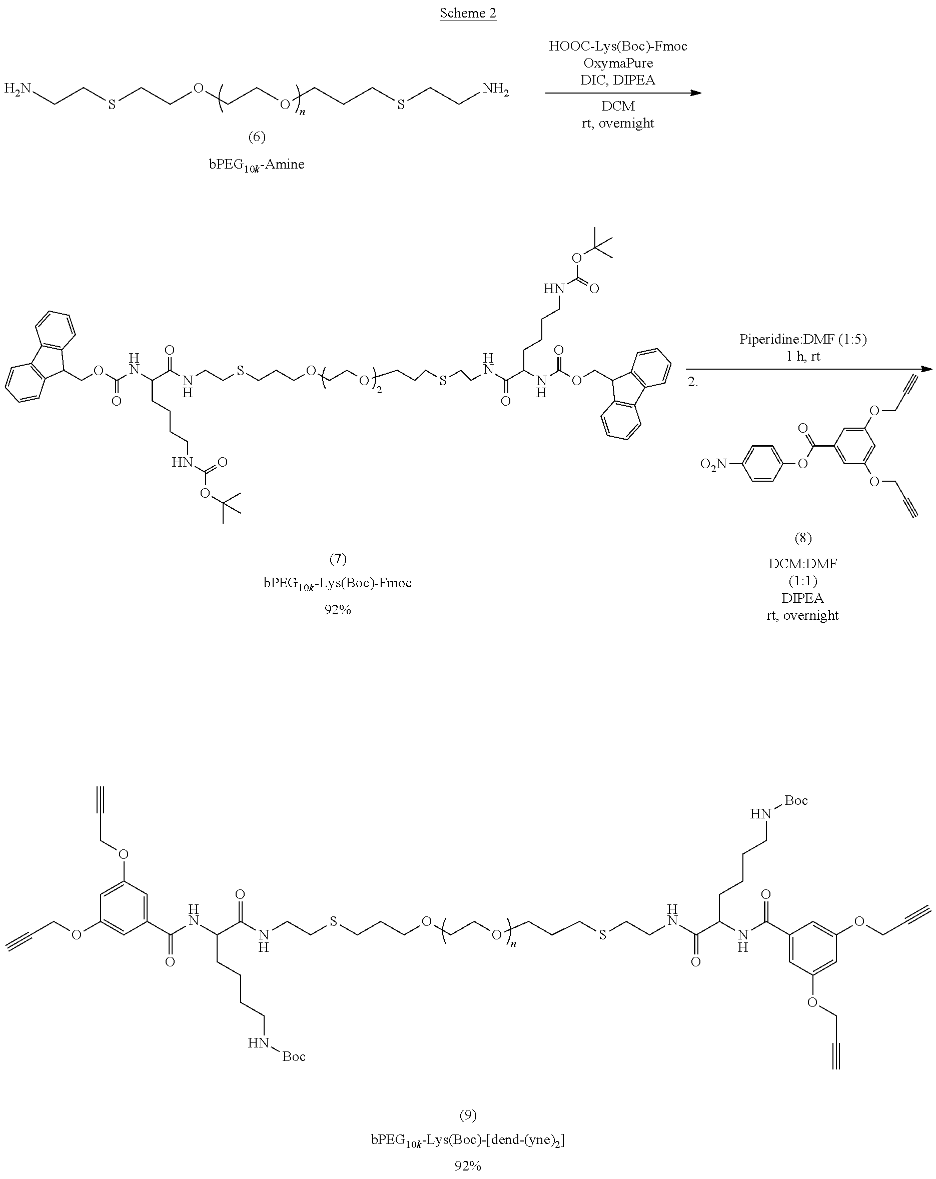

Fmoc-Lys(Boc)-CO₂H (6 eq.) and DIC (6 eq.) were dissolved in DCM (2 mL) followed by addition of Oxyma Pure (6 eq.) and DIPEA (6 eq.). 600 mg (0.058 mmol) of bPEG₁₀ₖ-Amine (6) were added to the solution and the reaction mixture was stirred overnight at room temperature. The crude mixture was loaded on a MeOH based LH20 SEC column. The fractions that contained the product, bPEG_{10kDa}-Lys(Boc)-Fmoc (7), were unified, and MeOH was evaporated in vacuum. Further purification was performed by re-dissolving the oily residue in DCM (1 mL) followed by precipitation by the dropwise addition of ether (50 mL). The white precipitate was filtered, washed twice with ether, and dried under a high vacuum. The product (7) was obtained as a white solid (92% yield).

¹H-NMR (CDCl₃): δ 7.69 (d, J=7.5 Hz, 4H, Ar—H), 7.53 (d, J=7.2 Hz, 4H, Ar—H), 7.33 (t, J=7.4 Hz, 4H, Ar—H), 7.24 (d, J=7.4 Hz, 41-1, Ar-1), 6.60 (i, 211, —CH₂—NH—CO—CH—), 5.59 (m, 2H, —NH-Fmoc), 4.71 (m, 2H, —NH-Boc), 4.35 (d, J=6.2 Hz, 4H, Fmoc-CH₂—), 4.14 (t, J 6.6 Hz, 21H, Fmoc-CH—CH₂—), 4.05 (n, 21-1, —CO—CH—NH—), 3.57-3.31 (m, PEG backbone), 3.10-2.86 (m, 4H, Boc-NH—CH₂—), 2.64-2.49 (m, 8H, —CH₂—S—CH₂—), 1.88-1.49 (m, 8H, —O—CH₂—CH₂—CH₂—S—+Boc-NH—(CH₂)₃—CH₂—CH—), 1.36 (m, 26H, Boc-NH—CH₂—CH₂—CH₂—CH₂—CH—+Boc). ¹³C-NMR (100 MHz, CDCl₃): δ 171.6, 156.2, 143.8, 141.3, 127.7, 127.1, 125.0, 120.0, 70.6, 70.1, 69.4, 66.9, 54.9, 47.2, 39.9, 38.5, 32.1, 31.5, 29.6, 28.4, 28.2, 22.5. GPC (DMF+25 mM NH4Ac): calculated Mn=11.1 kDa, experimental Mn=11.7 kDa, DM=1.06.

400 mg (0.036 mmol) of bPEG_{10kDa}-Lys(Boc)-Fmoc (7) were dissolved in 20% piperidine v/v in DMF (3 mL) and stirred for 1 hour. The deprotected product was precipitated by the dropwise addition of ether (50 mL). The white precipitate was filtered, washed with ether, and dried under a high vacuum. The deprotected product was obtained as a white solid. Compound (8) (10 eq.), prepared as reported in Harnoy et al., (Chem. Soc. 2014, 136, 7531-7534), the contents of which are hereby incorporated by reference in their entirety, and HBTU (10 eq.) were dissolved in DCM:DMF 1:1 (1 mL) followed by the addition of DIPEA (20 eq.). The solution was added to the deprotected bPEG_{10kDa}-Lys(Boc)-NH₂ dissolved in DCM (1 mL). The reaction was stirred overnight. Complete coupling was confirmed by a negative Kaiser test. The crude mixture was loaded on a MeOH based LH20 SEC column. The fractions that contained the product, bPEG_{10kDa}-Lys(Boc)-[dend-(yne)₂](9), were unified, MeOH was evaporated in vacuum, and the product was further dried under a high vacuum. The product (9) was obtained as a white solid (92% yield).

¹H-NMR (CDCl₃): δ 7.07 (d, J=2.1 Hz, 4H, Ar—H), 6.97 (d, J=7.4 Hz, 2H, —CH—NH—CO—Ar—), 6.85-6.70 (m, 4H, Ar—H+—CH₂—NH—CO—CH—), 4.72 (d, J=2.2 Hz, 10H, —O—CH₂—C≡CH+—NH-Boc), 4.56 (q, J=7.6 Hz, 2H, —CO—CH—NH—), 3.82-3.33 (m, PEG backbone), 3.18-3.02 (m, 4H, Boc-NH—CH₂—), 2.74-2.53 (m, 10H, —CH₂—S—CH₂—+—O—CH₂—(C≡CH), 2.08-1.68 (m, 8H, —O—CH₂—CH₂—CH₂—S-+Boc-NH—(CH₂)₃—CH₂—CH—), 1.58-1.35 (m, 26H, Boc-NH—CH₂—CH₂—CH₂—CH₂—CH-+Boc). ¹³C-NMR (CDCl₃) δ 171.4, 166.6, 158.6, 156.0, 136.1, 106.8, 105.6, 81.9, 76.0, 70.4, 69.3, 56.1, 53.4, 41.8, 38.5, 32.1, 31.4, 29.6, 29.5, 28.3, 28.1, 22.6. GPC (DMF+25 mM NH4Ac): calculated Mn=11.1 kDa, experimental Mn 12.3 kDa, DM=1.06.

The synthesis route for the preparation of bPEG₁₀ₖ-Lys(Cy3)-[dend-(hexanoate)₄] is shown in Scheme 3.

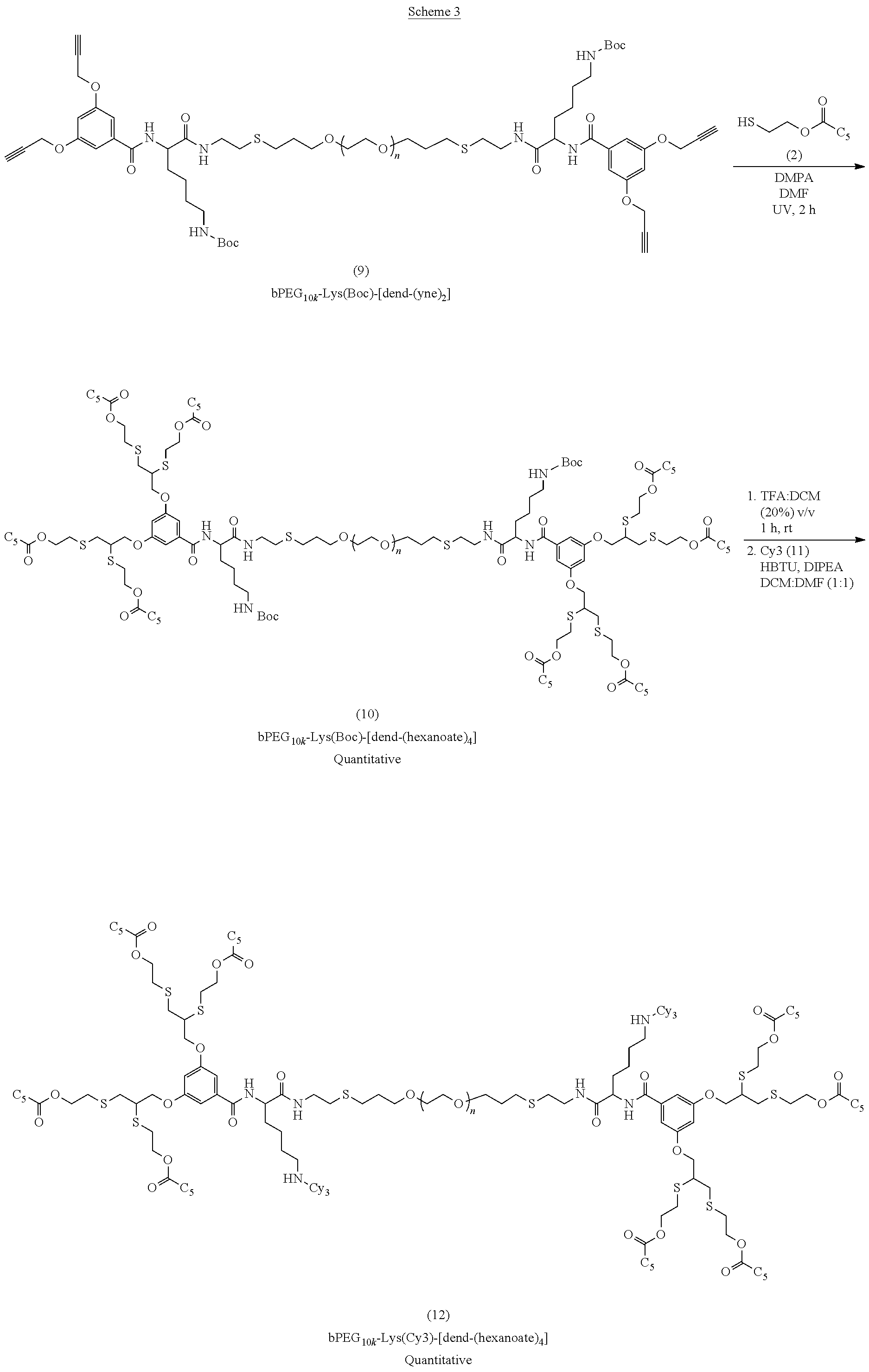

200 mg of compound (9) (0.036 mmol) were dissolved in DMF (0.5 mL). Compound (2) (254 mg, 1.44 mmol) and DMPA (3.7 mg, 0.014 mmol) were added to the solution. The solution was purged with nitrogen for 15 minutes and then placed under UV light at 365 nm for 2 hours. The crude mixture was loaded on a MeOH based LH20 SEC column. The fractions that contained the product, bPEG-Lys(Boc)-[dend-(hexanoate)₄](10) were unified, and MeOH was evaporated in vacuum. Further purification was performed by re-dissolving the oily residue in DCM (1 mL) followed by precipitation by the dropwise addition of ether (50 mL). The white precipitate was filtered, washed twice with ether, and dried under a high vacuum. The product (10) was obtained as a white solid (quantitative yield).

¹H-NMR (CDCl₃): δ 7.02 (d, J=7.5 Hz, 2H, —CH—NH—CO—Ar—), 6.98 (d, J=2.8 Hz, 4H, Ar—H), 6.68 (n, 2H, —CH₂—NH—CO—CH—), 6.60 (d, J=2.6 Hz, 2H, Ar—H), 4.68 (m, 2H, —NH-Boc), 4.54 (q, J 7.3 Hz, 2H, —CO—CH—NH—), 4.33-4.12 (m, 24H, —Ar—O—CH₂—+CH₂—O—CO—C₅), 3.83-3.42 (m, PEG backbone), 3.20 (q, J=5.5 Hz, 4H, —CH—S—), 3.17-2.54 (m, 36H, Boc-NH—CH₂+—CH—CH₂—+—CH—CH₂—S—CH₂—+—NH—CH₂—C₂—S—+—CH₂—H—S—), 2.28 (t, J=7.5 Hz, 16H, —O—CO—CH₂), 2.04-1.66 (m, 8H, —O—CH₂—CH₂—CH₂—S—+Boc-NH—(CH₂)₃CH₂—CH₂), 1.54-1.38 (m, 2611, Boc-NH—CH₂—CH₂—CH₂—CH₂—CH—+Boc), 1.35-1.15 (m, 48H, —O—CO—CH₂—(CH₂)₃—CH₃), 0.86 (t, J=6.3 Hz 24H, —O—CO—(CH₂)₄—CH₃). ¹³C-NMR (CDCl₃) δ 173.5, 171.4, 166.6, 159.4, 156.0, 136.0, 106.1, 104.6, 70.4, 63.3, 63.0, 53.4, 45.4, 40.0, 38.5, 34.7, 34.1, 32.0, 31.4, 31.1, 30.3, 29.5, 28.3, 28.1, 24.5, 22.7, 22.2, 13.8. GPC (DMF+25 mM NH₄Ac): calculated Mn=12.5 kDa, experimental Mn=13.7 kDa, DM 1.08.

Cy3 fluorescent dye (11) was synthesized as reported in Jung et al., (Bioorganic and Medicinal Chemistry 2006, 15, 92-97), the contents of which are hereby incorporated herein in their entirety.

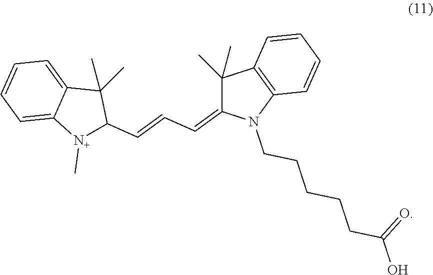 [SMILES: C[NH+]1c2ccccc2C(C)(C)C1/C=C/C=C1/N(CCCCCC(=O)O)c2ccccc2C1(C)C]

100 mg (0.008 mmol) of compound (10) was dissolved in DCM (1 mL) and TFA (200 μL). The mixture was allowed to stir for 1 h and the reaction was monitored via 1-PLC. Once the Boc deprotection was confirmed by HPLC, DIPEA was added to the reaction mixture until fume release was terminated. Then, a MeOH-based LH20 SEC column was performed to discard the TFA.

Cy3 (29.3 mg, 0.048 mmol), HBTU (18.2 ng, 0.048 mmol) and DIPEA (31 mg, 0.239 mmol) were dissolved in a total volume of 1 mL DCM:DMF (1:1 v/v) in a 4 mL vial. The solution was stirred for 2 minutes and added to the concentrated polymer hybrid purified by a MeOH-based LH20 SEC column. The reaction mixture was allowed to stir for 2 hours and then loaded on a MeOH-based LH20 SEC column. The fractions containing the product, bPEG-Lys(Cy3)-[dend-(hexanoate)₄](12) were unified, MeOH was evaporated, and the product was further dried under a high vacuum. The product (12) was obtained as a dark blue solid (quantitative yield).

¹³C-NMR (CDCl₃): 173.5, 166.4, 159.3, 150.7, 141.9, 136.5, 129.0, 128.8, 125.5, 125.4, 122.0, 111.2, 111.0, 106.4, 105.1, 104.5, 104.0, 70.4, 63.4, 63.1, 58.8, 54.1, 49.0, 45.6, 44.6, 39.2, 38.7, 38.0, 37.8, 36.0, 34.8, 34.0, 31.5, 31.2, 31.0, 30.2, 29.5, 28.2, 28.1, 26.7, 26.0, 25.0, 24.5, 22.7, 22.2, 13.8.

### Example 2

### Characterization of the Di- and Tri-Block Amphiphiles

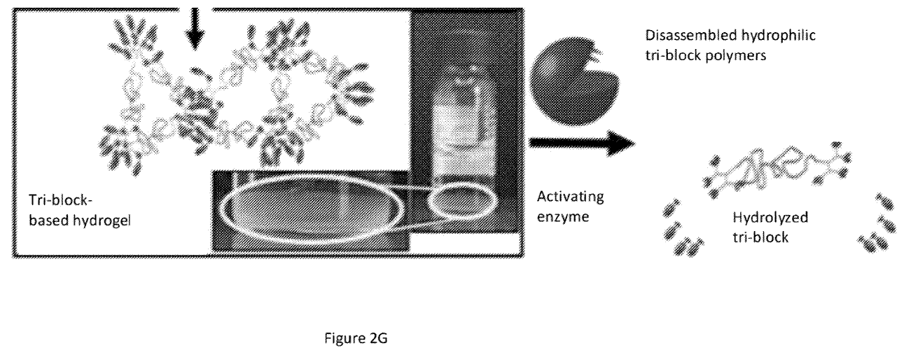

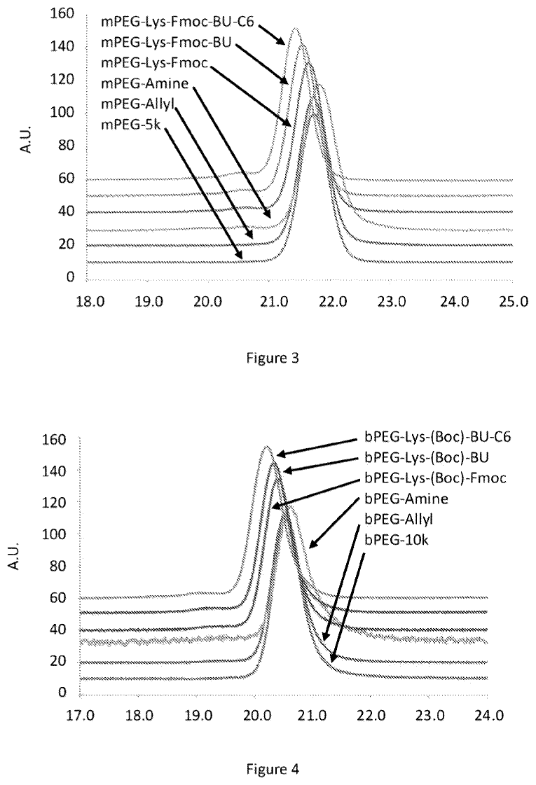

The DBAs and TBAs of Example 1 were characterized by Gel Permeation Chromatography (GPC) (Malvern Viscotek GPCmax) using the following parameters: Columns: 2×PSS GRAM 1000 Å+PSS GRAM 30 Å; Columns temperature: 50° C.; Flow rate: 0.5 ml/min; Injection time: 60 min; Injection volume: 50 μL from a 10 mg/ml sample; Diluent+mobile phase: DMF+25 mM NH₄Ac; Needle wash: DMF; Detector: Viscotek VE3580 RI detector. The amphiphiles were directly dissolved in the diluent to a final concentration of 10 mg/mL and filtered using a 0.22 μm PTFE syringe filter. The spectra are shown in FIGS. 3 and 4.

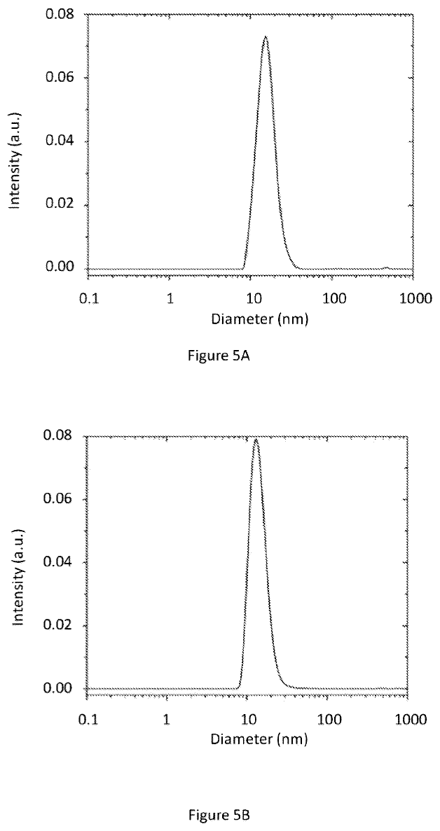

The DBAs and TBAs were further characterized by Dynamic Light Scattering (DLS). Compounds (3) and (10) were dissolved in a phosphate buffer (pH 7.4) at different ratios. FIGS. 2C and 5A show the DLS data for compounds (3) and (10) at a 1:1 ratio with a total final concentration of 5 mg/mL. FIG. 5B shows the DLS data for compounds (3) and (10) at a 2:1 ratio with a total final concentration of 7.5 mg/mL. Micelles having sizes of about 20 nm were formed. The solutions were sonicated for 15 minutes and filtered through a 0.22 μm nylon syringe filter. Measurements were performed at t=0 before the addition of PLE enzyme.

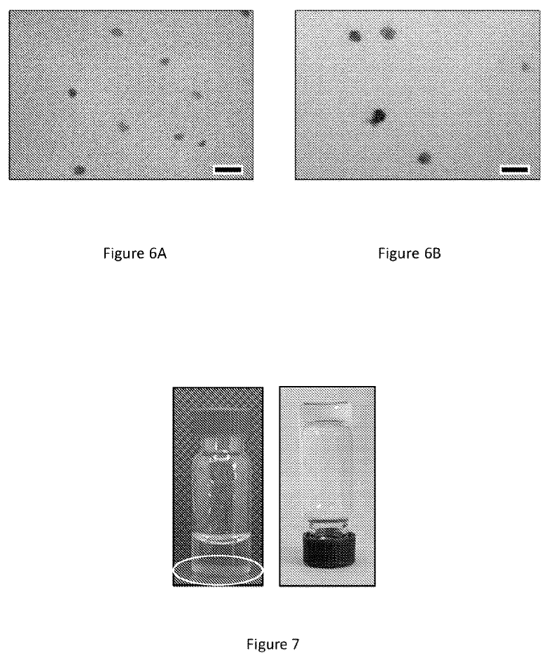

Additional characterization was performed by Transmission Electron Microscopy (TEM). Compounds (3) and (10) were dissolved in a phosphate buffer (pH 7.4) at different ratios. FIGS. 2D and 6A show TEM images of micelles formed from compounds (3) and (10) at a 1:1 ratio with a total final concentration of 10 mg/mL. FIG. 6B shows a TEM image of micelles formed from compounds (3) and (10) at a 2:1 ratio with a total final concentration of 15 mg/mL. The solutions were sonicated for 15 minutes and filtered through a 0.22 μm nylon syringe filter. Measurements were performed at t=0 before the addition of PLE enzyme.

The TBA compound (10) was further assessed for its ability to form a hydrogel in water. FIG. 7 shows images of gels prepared from compound (10) in water using thin-film hydration (left) and solvent exchange (right) method.

### Example 3

### Enzymatic Cleavage

A clear micellar solution (1:1 DBA:TBA ratio) was prepared by mixing 5 mg of each compound (3) and (10) in 1 mL PBS providing a total concentration of 10 mg/mL (FIG. 2B). Similarly, for the 2:1 DBA:TBA ratio, 10 mg of compound (3) and 5 mg of compound (10) were mixed in 1 mL PBS providing a total concentration of 15 mg/mL. The vials were vortexed until full solubility was obtained and then placed in an ultrasonic bath for 15 minutes. PLE was added to yield a final concentration of 0.36 μM and degradation was followed at 37° C. by monitoring the area under the peak of the parent amphiphile and hydrolyzed polymer by HPLC at 297 nm. Each experiment was conducted thrice and the reported values at each time point are the mean value and the standard deviation is the error.

When using a micellar solution containing a 1:1 ratio of DBA:TBA, two peaks corresponding to the two types of amphiphiles with the 1:1 ratio were observed (FIG. 2E). A significantly higher selectivity of the enzyme towards degradation of the DBA, which reached 70% after 5 hours, was observed, while the TBA remained nearly intact (~10% degradation). Thereafter, a sudden drop of about 50% in the area of the TBA peak after 5 hours, followed by a further decrease in the concentrations of both types of amphiphiles were detected (FIG. 2F). The decrease was attributed to the formation of a hydrogel (FIG. 2G).

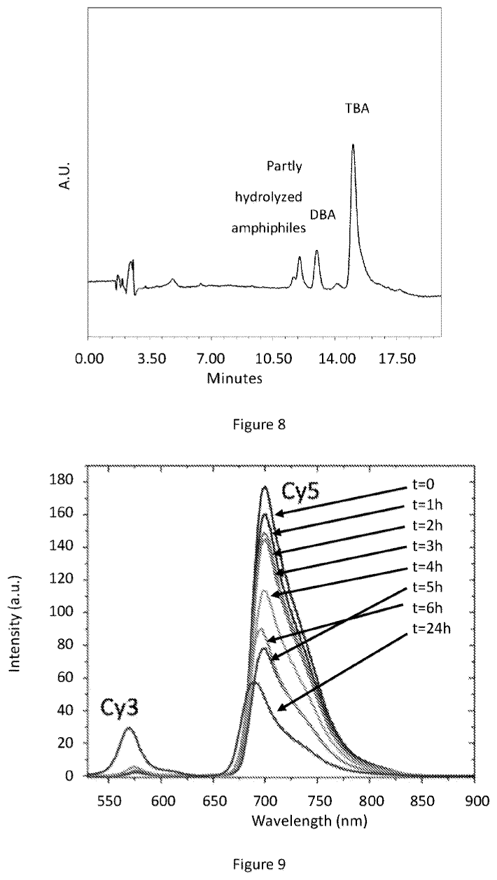

In order to analyze the composition of the formed hydrogel, the buffer solution above the hydrogel was removed and the remaining hydrogel was washed three times with PBS and then dissolved in acetonitrile. The HPLC analysis showed the presence of 11% partly hydrolyzed amphiphiles, 12% DBA and 77% TBA (FIG. 8). The presence of the TBA and a low amount (~10%) of non-hydrolyzed DBA corresponded well with the second drop in DBA and TBA concentrations upon gelation.

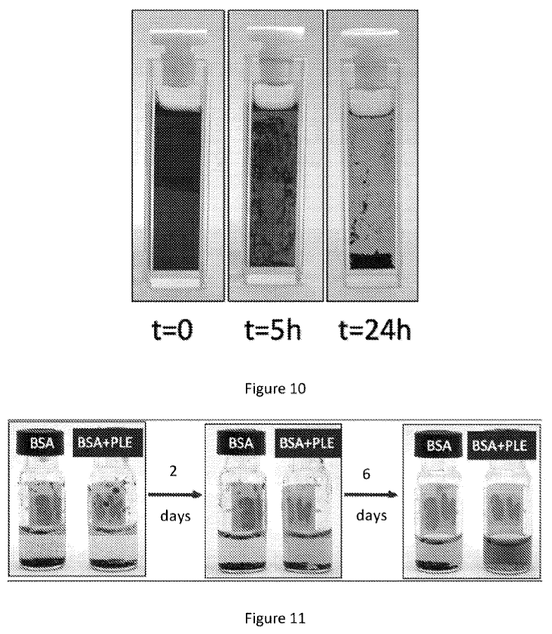

The amphiphiles were then labeled with fluorescent markers that can form Forester resonance energy transfer (FRET; Teunissen et al., Chem. Soc. Rev. 2018, 47 (18), 7027-7044). DBA hybrids were labeled with Cy5 as a FRET acceptor, and TBA hybrids were labeled with Cy3 as a donor. A micellar solution (1:1 DBA:TBA) was prepared by mixing 4.5 mg of each compound (3) and (10) and 0.5 mg of each compound (5) and (12) in 1 mL PBS to a total concentration of 10 mg/mL. The vials were vortexed until full solubility was obtained and then placed in an ultrasonic bath for 15 minutes. PLE was added to yield a final concentration of 0.36 μM and fluorescence was measured at 37° C. by exciting at 512 nm (Cy 3 excitation). The emission was measured at 570 nm for Cy3 and 700 nm for Cy 5. The results showed a very strong FRET signal at 700 nm, providing vital evidence for the co-assembly (FIG. 9). The labeled co-assembled micelles were then incubated with the activating enzyme. The spectra showed a decrease in the FRET signal, which is attributed to the enzymatic hydrolysis of DBA as the hydrolyzed DBA have substantially higher hydrophilicity which results in their diffusion thereby rendering them less available for FRET inside the hydrophobic domains of the assembled structures. In addition, when visualizing the vial, the sample initially showed a clear purple solution that was attributed to the presence of both dyes in the mixed co-assembled micelles. Then, following the addition of PLE, a hydrogel formed which maintained the purple color due to the presence of both dyes in the aggregated hydrogel. However, the solution became bluer due to the change in the ratio of Cy5 and Cy3 amphiphiles, in comparison with the initial conditions (FIG. 10).

In order to test the ability of TBA-based hydrogel to undergo further enzymatic degradation and transform into soluble hydrophilic tri-block polymers, bovine serum albumin (BSA) was added to the hydrogels. The solution above the hydrogel was removed and the remaining hydrogel was washed three times with PBS. Two parallel experiments were conducted, first, 500 μL of 3.5 mg/mL of BSA in PBS was added and second, 500 μL of 3.5 mg/mL of BSA along with 1 μM of PLE in PBS were added. BSA was added at a 3-fold higher concentration than the concentration of PLE due to the rather slow enzymatic degradation of TBA at the micellar state. Images of the vials showed the stability of the hydrogel in the presence of BSA and its full degradation in the presence of BSA+PLE into soluble hydrolyzed polymers, yielding a clear purple solution due to the presence of both Cy-3 labeled and Cy-5 labeled polymers (FIG. 11).

### Example 4

### Cargo Release by Enzymatic Degradation

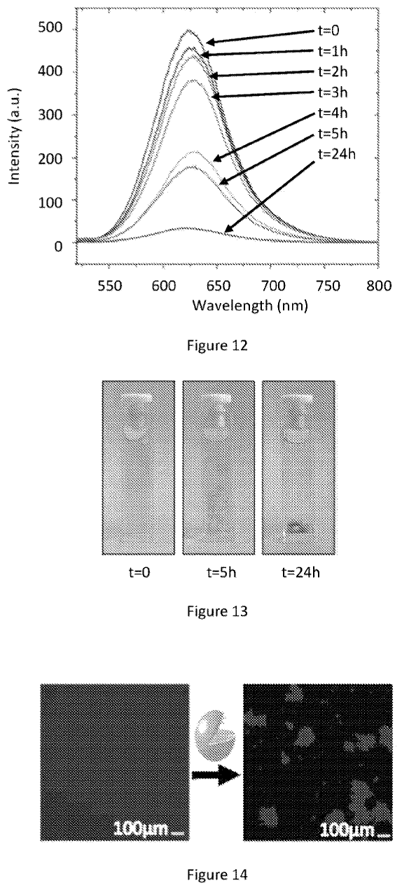

To examine the release pattern of an encapsulated cargo, a Nile red was used as a model cargo. A micellar solution (1:1 DBA:TBA) was prepared by mixing 5 mg of each compound (3) and (10) and Nile red in 1 mL PBS providing a total polymeric concentration of 10 mg/mL and 10 M of Nile red. The sample was vortexed until full solubility was obtained and then placed in an ultrasonic bath for 15 minutes. PLE was added to yield a final concentration of 0.36 μM and degradation was followed at 37° C. by measuring the fluorescence by excitation at 500 nm. The formed micelles and hydrogels were assessed using fluorescence spectroscopy and confocal microscopy. The initial micellar solution showed a Nile-red emission at ~640 nm (FIG. 12). Following the addition of PLE, a slow decrease in fluorescent emission was observed during the first three hours, followed by a sudden drop at around 4 h, which was attributed to the precipitation of the hydrogel as can be seen in the 5 hours image (FIG. 13, middle image). Eventually, after 24 h, the concentration of dyes in the solution dropped to almost zero, indicating the efficient encapsulation and retention of the cargo molecules during the mesophase transition. The confocal microscope images (FIG. 14) showed diffuse fluorescence of the entire micellar sample as the individual micelles were too small to be directly observed. This diffused emission shifts into localized hydrogel aggregates with strong emission due to the concentrating effect of the dyes upon the enzymatically induced gelation, thereby demonstrating the potential of using such programable formulation that can transform from micellar nanocarriers into drug-depots.

### Example 5

### Enzymatic Cleavage at Different Time Frames

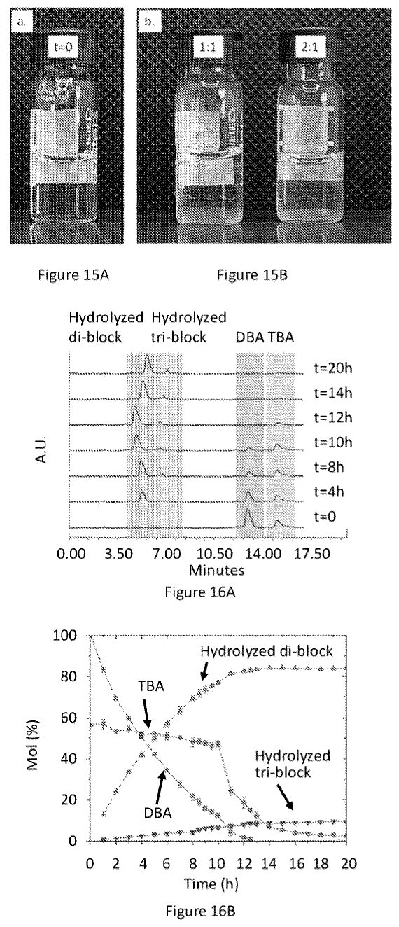

After confirming the transformation of the micelles into hydrogels, which then slowly transform into soluble polymers, the ability to induce a slower transformation from micelles to hydrogels was evaluated. The concentration of the DBA was therefore increased thereby prolonging the time duration for achieving the critical concentration of degraded DBA that enables the transition to hydrogels. Doubling the concentration of DBA, did not significantly affect the size of the micelles (FIGS. 5B and 6B). FIGS. 15A-15B show the gel formed after the disassembly of the micelles due to enzymatic degradation of two experiments. In the first experiment, compounds (3) and (10) were present in the assembly at a 1:1 ratio with a total final concentration of 10 mg/mL, while in the second experiment, compounds (3) and (10) were present in the assembly at a 2:1 ratio with a total final concentration of 15 mg/mL. The enzyme (PLE) concentration was 0.36 μM. As seen in the figures, the amount of compound (10) in the two different enzymatic experiments did not change thereby resulting in the same amount of gel formed at the bottom of the vials. While similar micelles and gel formation was achieved in both experiments, the transition to a hydrogel at a DBA:TBA ratio of 2:1 occurred only after 10 hours (FIGS. 16A-16B), nearly twice the time of the transition at the 1:1 ratio. Comparison of the two experiments showed that for both 1:1 and 2:1 formulations, the critical gelation ratios, at which the transition between the two mesophases occurred, had a nearly similar value of ~0.3:1 DBA:TBA. These results demonstrate the ability to tailor the time frame of the mesophase transitions by adjusting the composition of the formulation.

Taken together, these results demonstrate the ability to use molecular architecture as a tool for programming sequential mesophase transitions. Unexpectedly, despite the similar HLB and enzymatically cleavable groups of the DBA and TBA, the different architectures and molecular weights significantly affected the reactivity of the two types of amphiphiles towards enzymatic degradation. Upon enzymatic activation, DBA was selectively degraded and the DBA:TBA ratio decreased until the amount of DBA could no longer stabilize the micellar form and TBA aggregated to form hydrogels. Upon further incubation with the enzyme, the formed TBA-based hydrogels underwent slow transition into soluble polymers, most likely through an erosive degradation process.

### Example 6

### Polymeric Formulations Undergoing Multi-Step Mesophase Transitions

A variety of formulations that are capable of undergoing multiple mesophase transitions in response to multiple stimuli were designed. Amphiphilic PEG-dendron (DBA) hybrids based on a commercial 5 kDa mono-functional polyethylene glycol (mPEG) as a hydrophilic block and a dendron as the hydrophobic block were synthesized. Additionally, dendron-PEG-dendron tri-block amphiphiles (TBA) hybrids with different architectures were synthesized by using a 10 kDa PEG chain. Both the DBA and the TBA hybrids incorporated enzymatically responsive end-groups that serve as substrates for porcine liver esterase (PLE) by featuring ester functional groups or penicillin G amidase (PGA) by featuring amide functional groups. The TBA hybrids were also labeled with 7-(diethylamino) coumarin-3-carboxylic acid (7-DEAC) as a fluorescent tag.

The synthesis route for the preparation of the porcine liver esterase (PLE) substrate, 2-mercaptoethyl hexanoate, is shown in Scheme 4.

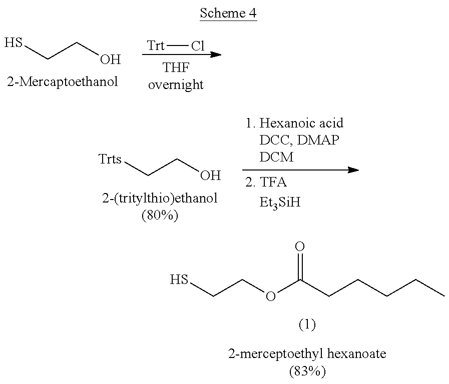

Trityl chloride (17.1 g, 61.4 mmol, 1.2 eq.) was dissolved in THF (40 mL), and 3-mercaptoethanol (3.6 mL, 51.2 mmol, 1.0 eq.) was added to the flask. The reaction was stirred overnight at room temperature. The solvent was then evaporated in vacuum and the product (3-(tritylthio) ethanol) was purified using flash silica chromatography (DCM:EtOAc, 90:10). The product was obtained as a white solid in 80% yield (13.1 g). 2-(tritylthio) ethanol (3.0 g, 9.4 mmol) and hexanoic acid (1.29 ml, 1.20 g, 10.3 mmol) were dissolved in 20 ml DCM. DCC (2.12 g, 10.3 mmol) was then added followed by DMAP (0.34 g, 2.8 mmol). The reaction was stirred at room temperature for about 4 h and the reaction mixture was filtered through a filter paper. 12 ml of TFA were added to the filtrate followed by triethylsilane addition (1.94 ml, 1.41 g, 12.17 mmol). The reaction was stirred for 30 minutes at ambient temperature and solvents were evaporated to dryness under vacuum. The product, (2-mercaptoethyl hexanoate), was purified using flash silica chromatography (Hex:DCM, 1:1). The product (identified on TLC using KMnO₄) was obtained as a colorless oil in 83% yield (1.37 g).

The synthesis route for the preparation of the penicillin G amidase (PGA) substrate (HS-PhAcAm) was performed as described in Harnoy et al. (Synlett. 2018, 29(19), 2582-2587), the contents of which are hereby incorporated herein in their entirety, and is shown in Scheme 5.

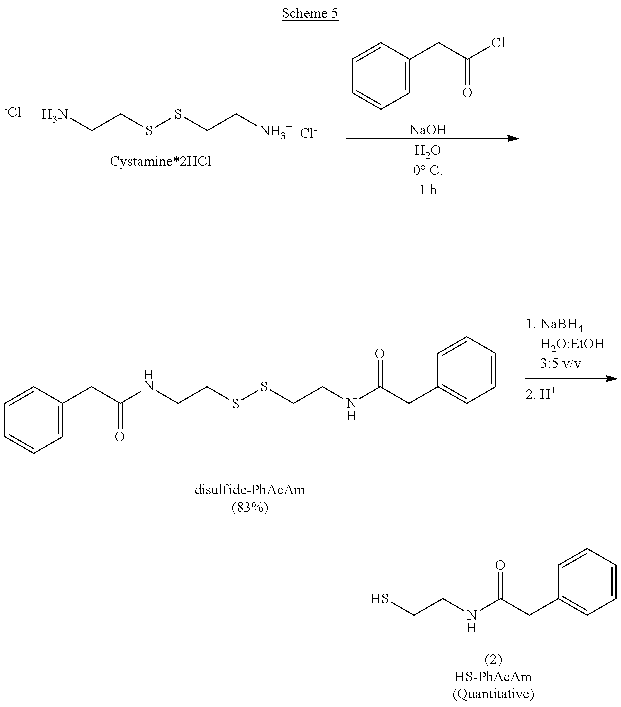

The synthesis route for the preparation of Ester-DBA and Amide-DBA is shown in Scheme 6.

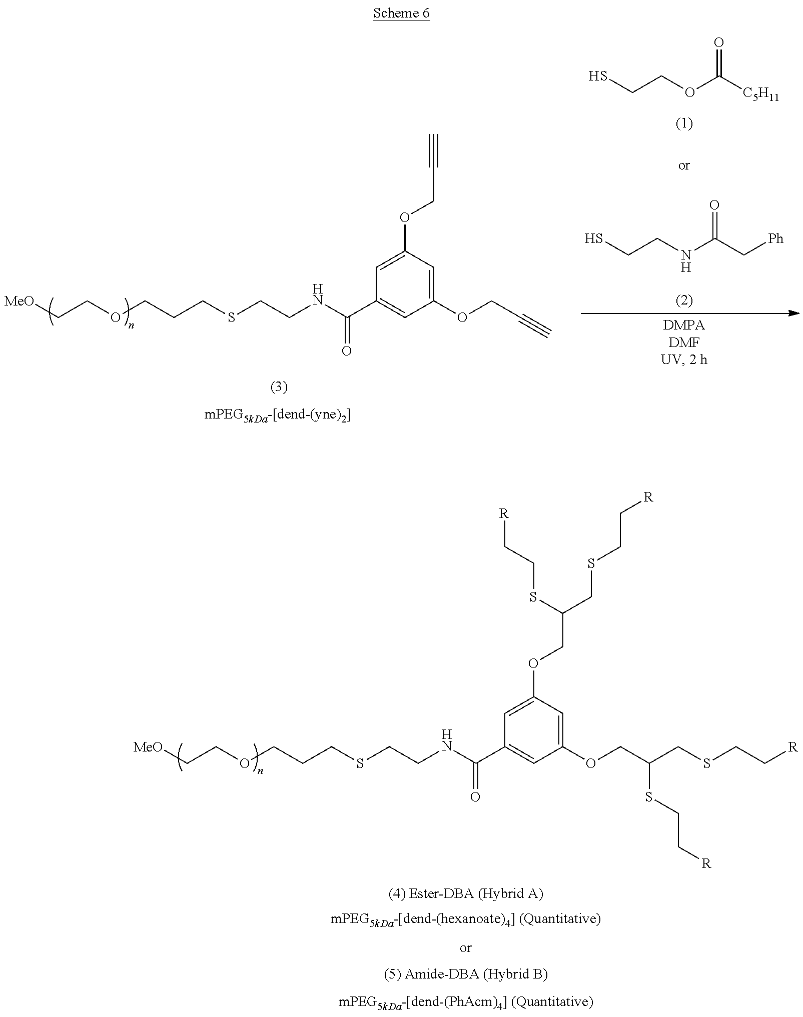

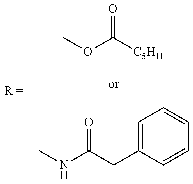

mPEG₅ₖ-[dend-(yne)₂] was synthesized as described in Harnoy et al. (Biomacromolecules 2017, 18(4), 1218-1228), the contents of which are hereby incorporated herein in their entirety. The reaction of mPEG₅ₖ-[dend-(yne)₂] with thiol functionalized enzymatic end groups was performed as follows. mPEG₅ₖ-[dend-(yne)₂](1 eq.), thiol (40 eq.), and DMPA (0.4 eq.; 1 mol % with respect to the thiol) were dissolved in DMF (0.5 mL per 100 mg of hybrid). The solution was purged with nitrogen for 20 minutes and then stirred under UV light (365 nm) for 2 hours. The reaction mixture was then loaded on a MeOH-based 1120 (Sephadex®) size exclusion column. Fractions that contained the product (identified by UV light and/or coloring with iodine) were unified, the organic solvents were evaporated to dryness, and the white solid was dried under a high vacuum.

The synthesis route for the preparation of coumarin labeled Ester-TBA, bPEG₁₀ₖ-bis-Lys(Coum.)-[dend-(hexanoate)₄], and coumarin labeled Amide-TBA, bPEG₁₀ₖ-bis-Lys(Coum.)-[dend-(PhAcAm)₄], is shown in Scheme 7.

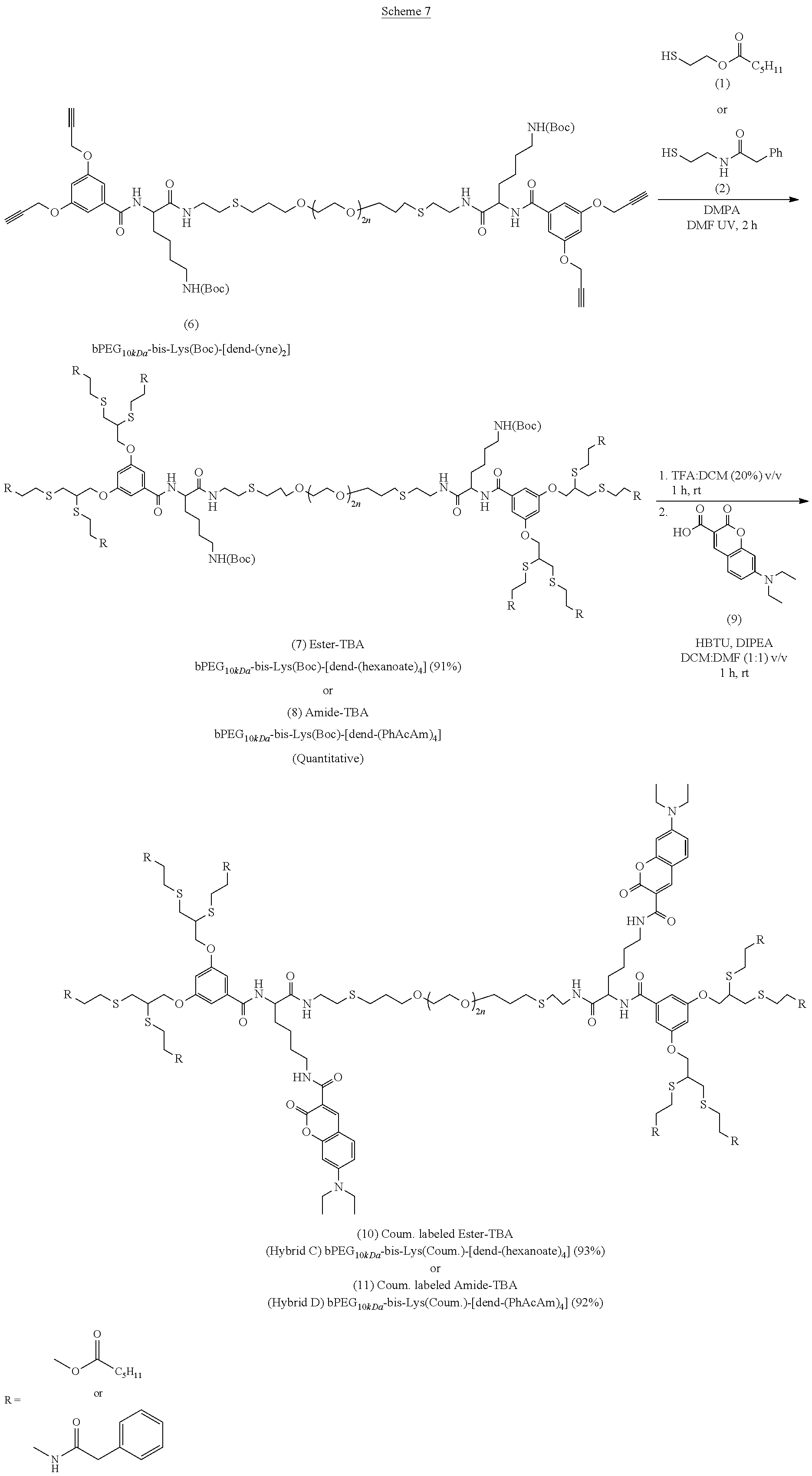

bPEG₁₀ₖ-bis-Lys(Boc)-[dend-(yne)₂] was synthesized as described in Rathee et al. (ACS Macro Lett., 2023, 12(6), 814-820), the contents of which are hereby incorporated herein in their entirety. Boc protected TBA hybrids were prepared as follows. bPEG₁₀ₖ-bis-Lys(Boc)-[dend-(yne)₂] (1 eq.), thiol (80 eq.) and DMPA (0.8 eq.; 1 mol % with respect to the thiol) were dissolved in DMF (0.5 mL per 100 mg of hybrid). The solution was purged with nitrogen for 20 minutes and then stirred under UV light (365 nm) for 2 hours. The reaction mixture was then loaded on a MeOH-based LH20 (Sephadex©) size exclusion column. Fractions that contained the product (identified by UV light and/or coloring with iodine) were unified, the organic solvents were evaporated, and the product was further dried under a high vacuum to obtain a white solid.

Coumarin labeled TBA hybrids were synthesized as follows. 200 mg (1 eq.) of Boc protected TBA compound were dissolved in DCM (2 mL) and TFA (400 μL) {TFA:DCM, 1:5}. The mixture was allowed to stir for 1 hour and the reaction was monitored via HPLC. Once the Boc deprotection was confirmed by HPLC, DIPEA was added to the reaction mixture until fume release was terminated. MeOH-based LH20 SEC column was performed to discard the TFA. The coumarin-acid compound (6 eq.), HBTU (6 eq.), and DIPEA (30 eq.) were dissolved in a total volume of 2 mL DCM:DMF (2:1 v/v) in a 4 mL vial. The solution was stirred for 2 minutes and added to the concentrated Boc deprotected polymer hybrid purified by a MeOH-based LH20 SEC column. The reaction mixture was allowed to stir for 2 hours and then loaded on a MeOH-based LH20 SEC column. The fractions containing the product were unified, MeOH was evaporated, and the product was further dried under a high vacuum. The coumarin labeled TBA product was obtained as a yellow solid.

The synthesized polymers and hybrids were characterized by NMR, high-performance liquid chromatography (HPLC), and size exclusion chromatography (SEC).

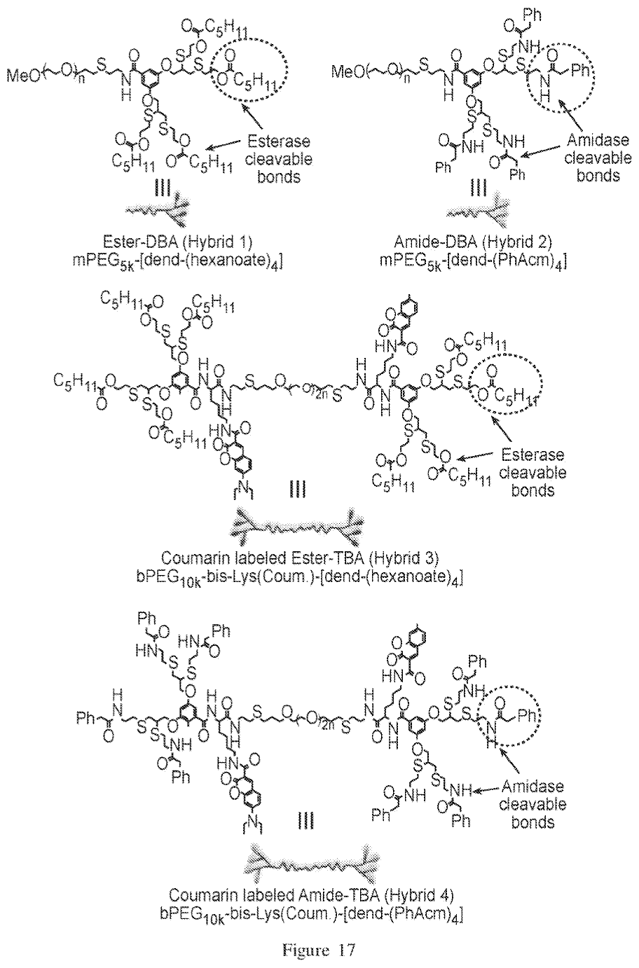

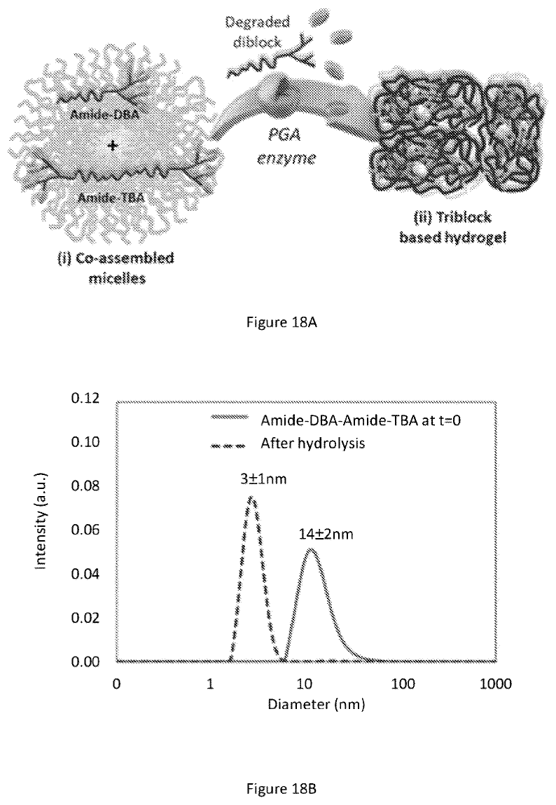

To evaluate the ability of the synthesized amphiphiles to co-assemble into mixed micelles, three different formulations were prepared: (1) Amide-DBA and Amide-TBA by mixing mPEG₅ₖ-[dend-(PhAcm)₄](Hybrid 2) and bPEG₁₀ₖ-bis-Lys(Coum.)-[dend-(PhAcm)₄](Hybrid 4) at a 1:1 ratio; (2) Amide-DBA and Ester-TBA by mixing mPEG₅ₖ-[dend-(PhAcm)₄](Hybrid 2) and bPEG₁₀ₖ-bis-Lys(Coum.)-[dend-(hexanoate)₄](Hybrid 3) at a 1:1 ratio; and (3) Ester-DBA and Amide-TBA by mixing mPEG₅ₖ-[dend-(hexanoate)₄](Hybrid 1) and bPEG₁₀ₖ-bis-Lys(Coum.)-[dend-(PhAcm)₄](Hybrid 4) at a 1:1 ratio (FIG. 17). The respective DBA and TBA hybrids were mixed at a 1:1 weight ratio in an organic solvent to obtain maximal blending, followed by evaporation of the solvent to yield a thin film. Hydration of the film using PBS buffer resulted in the desired formation of micelles with sizes of ≅12-16 nm as indicated by DLS.

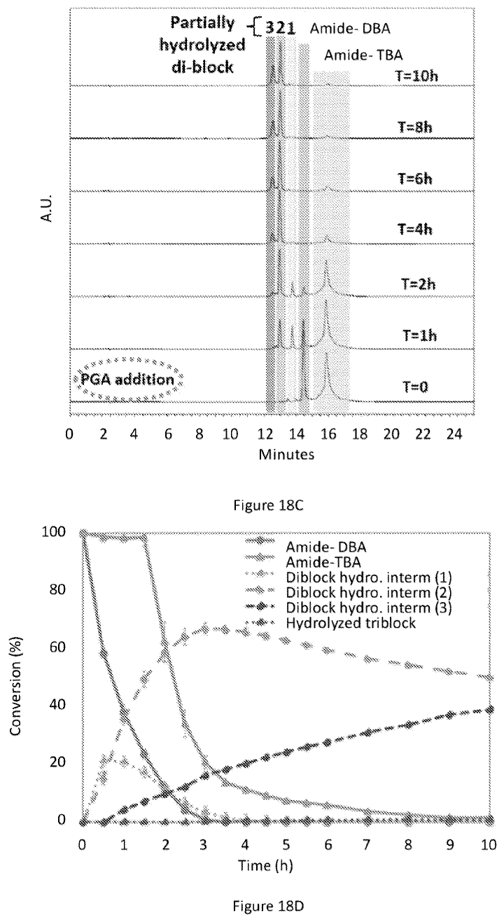

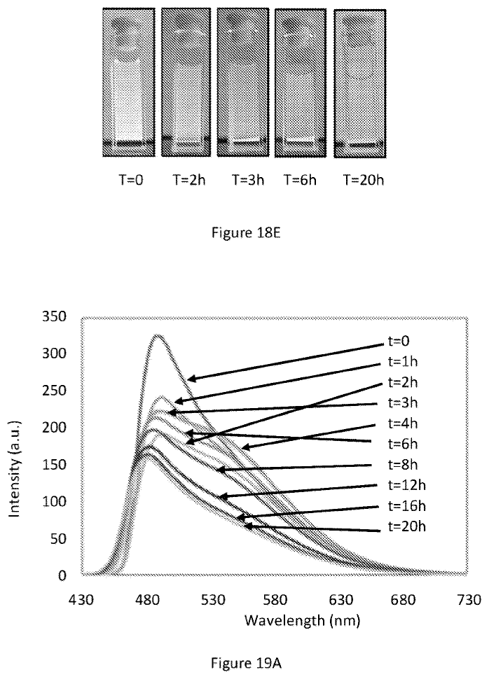

After confirming the self-assembly of these amphiphiles into mixed micelles, their response to enzymatic activity was examined using a combination of HPLC and absorption spectroscopy. HPLC was used to directly monitor the enzymatic degradation of the different hybrids that form the co-assembled micellar solution, whereas absorption spectroscopy was used to indirectly follow the disassembly of the micelles by monitoring the intensity of conjugated coumarin dye. First, the Amide-TBA and Amide-DBA formulation was studied (FIG. 18A). The formulation was exposed to PGA at t=0. DLS measurements of solutions of the formulation in the presence of PGA indicated a reduction in peak height associated with larger micellar aggregates, along with the emergence of a new peak corresponding to the smaller sizes of the non-assembled monomeric chains and the enzyme (FIG. 18B, dashed line). When studied using HPLC, initially two peaks corresponding to the two types of amphiphiles were observed. There appeared a high selectivity of the enzyme towards the degradation of the Amide-DBA, which reached nearly 76% after 1.5 h, while the TBA hybrid remained nearly intact (~2% degradation). Additionally, not all end-groups of Amide-DBA were cleaved, and the formation of partially cleaved intermediates was observed (FIG. 18C).

At t=2 h, a sudden drop of approximately 40% in the TBA concentration was observed (FIG. 18D). The chromatograms did not indicate an extensive simultaneous formation of cleaved tri-block, implying that the disappearance of Amide-TBA is not a result of enzymatic degradation but rather due to its aggregation into a hydrogel, as depicted in the images taken at different time points (FIG. 18E). This corresponds to the rapid decrease in Amide-TBA concentration.

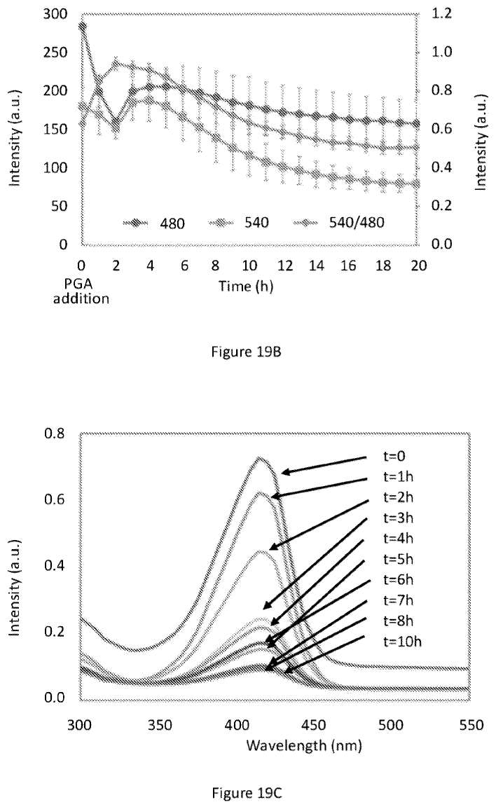

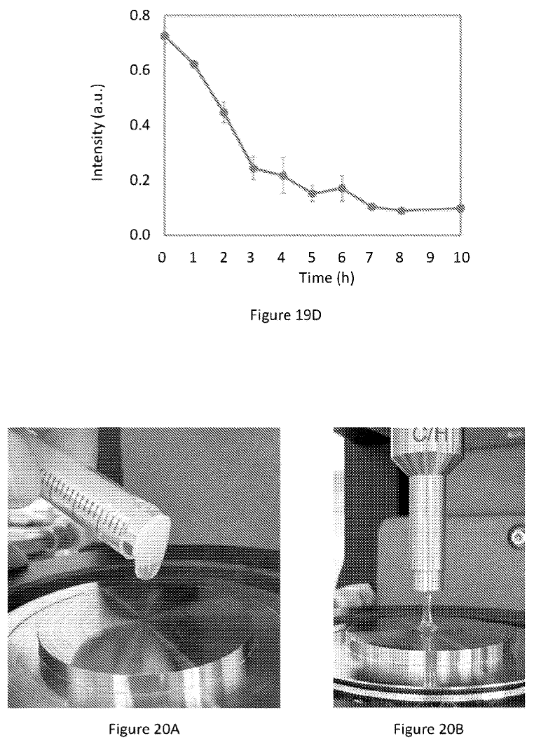

To further demonstrate the mesophase transition from micelles to a hydrogel, the fluorescence of the samples, utilizing a coumarin fluorescent marker attached to the Amide-TBA was monitored (FIGS. 19A-19B). Absorption spectra showed a strong signal at 415 nm which decreased upon the addition of the activating enzyme (PGA at 0.96 μM or 0.525 U/mL) to the mixed micellar formulation (FIG. 19C). The absorbance was decreased at a high rate within the initial 3 h (~70%), followed by a notably slower decrease thereafter (FIG. 19D). This observation is indicative of the gradual shift of the coumarin labeled Amide-TBA from micelles to a hydrogel, which precipitates due to the mesophase transition.

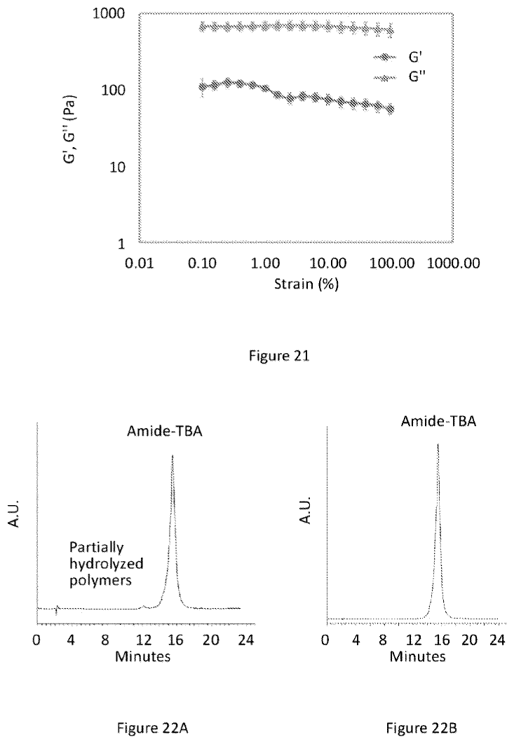

FIGS. 20A-20B show the formation of a viscous gel. To evaluate the mechanical attributes of the hydrogel, rheological measurements were performed. The amplitude sweep test did not indicate a gel-like behavior since the elastic modulus G′ was lower than the viscous modulus G″ (FIG. 21). This observation implies that the material is behaving in a predominantly viscous manner. Following rheological measurements, the gel was dissolved in acetonitrile, filtered, and studied using HPLC. The analysis showed the presence of Amide-TBA (FIGS. 22A-22B).

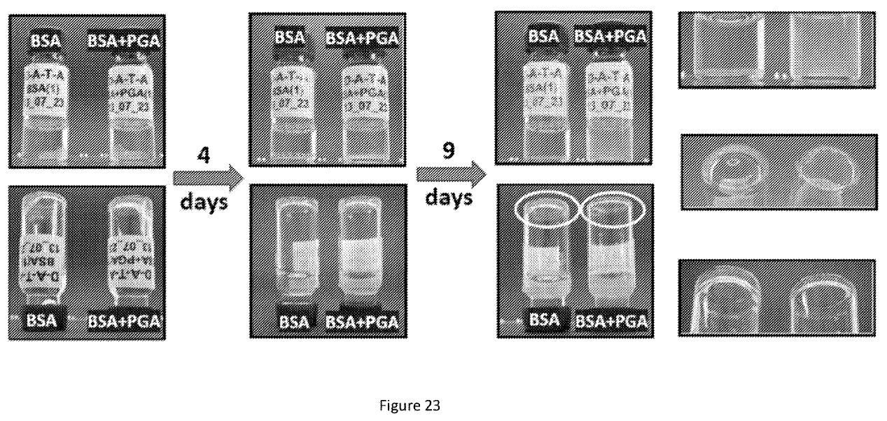

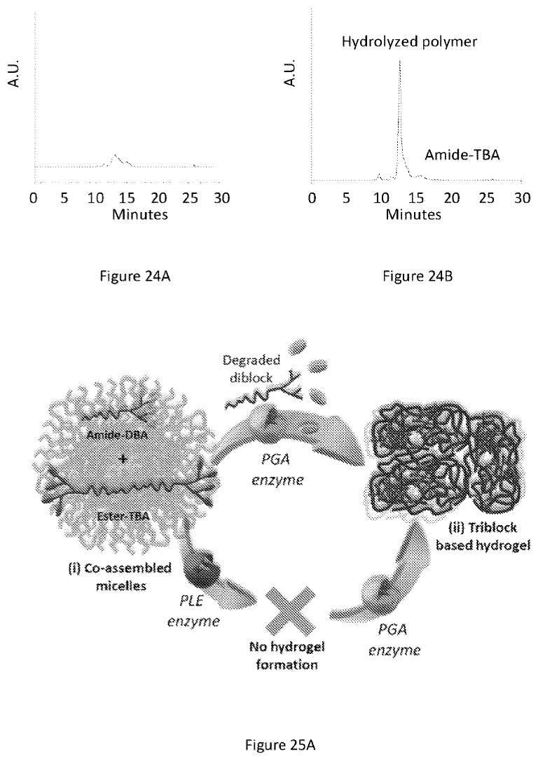

The TBA-based gel further underwent enzymatic degradation and transformation into soluble hydrophilic tri-block polymers and fragments thereof by exposure to PGA at a concentration that is 3.5-fold higher than the concentration used to invoke the transition from micelles to a hydrogel. In addition, bovine serum albumin (BSA) was added to the sample containing the enzyme and the control. The addition of BSA was performed in order to shift the equilibrium towards the unimer state and hence expedite the enzymatic hydrolysis. Images of the vials show the stability of the hydrogel in the presence of BSA and its full degradation into soluble hydrolyzed polymers in the presence of PGA (FIG. 23). To examine the composition of the degraded gel, a small sample of the hydrolyzed polymer solution was diluted with acetonitrile and analyzed by HPLC. The same protocol was followed for a gel containing BSA in order to determine if any degradation occurred under these conditions. FIGS. 24A-24B show the results of the HPLC analysis evidencing the formation of hydrolyzed polymer in the sample that was exposed to BSA and PGA.

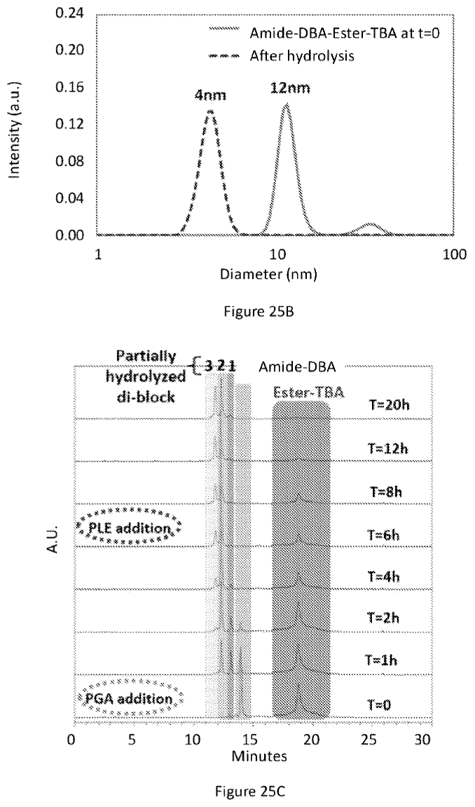

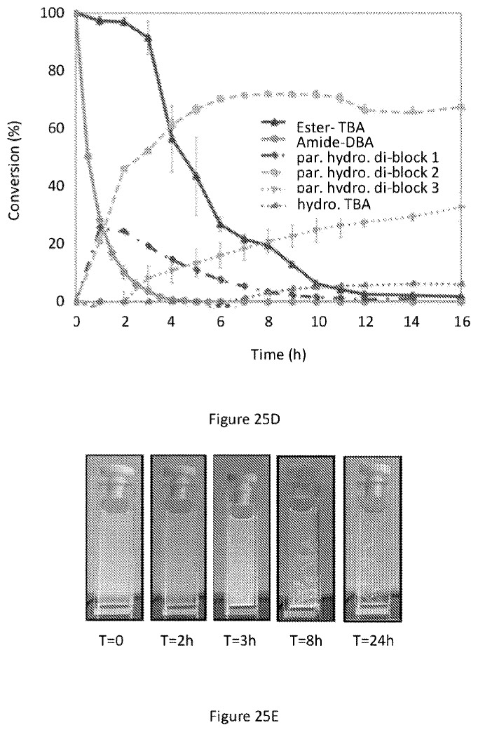

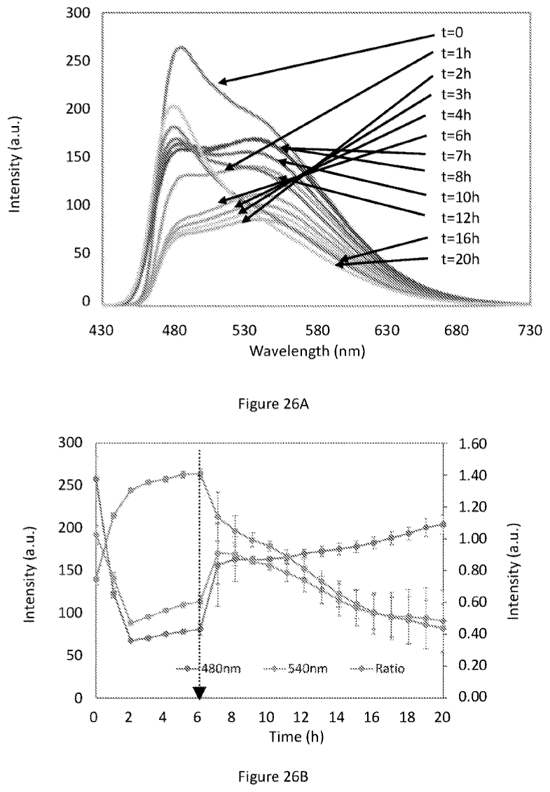

The formulation containing a mixture of Amide-DBA and Ester-TBA at a 1:1 ratio was further studied. In this formulation, the two transitions are invoked by two different enzymes. Using this formulation, the mesophase transitions were monitored by first introducing the PGA enzyme (0.96 μM or 0.525 U/mL) followed by the PLE enzyme (0.28 μM or 10 U/mL) (FIG. 25A) and vice versa. Upon incubation with PGA as the first activating enzyme, DLS measurements indicated the disappearance of the peak assigned to micelles with sizes of 12 nm (FIG. 25B, solid line) and the appearance of a peak at 4 nm assigned to smaller sizes of the non-assembled monomeric chains and the enzyme (FIG. 25B, dashed line). HPLC analysis showed the degradation of the Amide-DBA with high selectivity (FIG. 25C), which reached nearly 90% after 2 h, while the Ester-TBA remained nearly intact with approximately 4% degradation (FIG. 25D). At t=4 h, a sudden drop in the Ester-TBA concentration from 88% to 48% was observed. This drop in concentration is attributed to the aggregation of the Ester-TBA into a hydrogel, as depicted in the images taken at different time points (FIG. 25E).

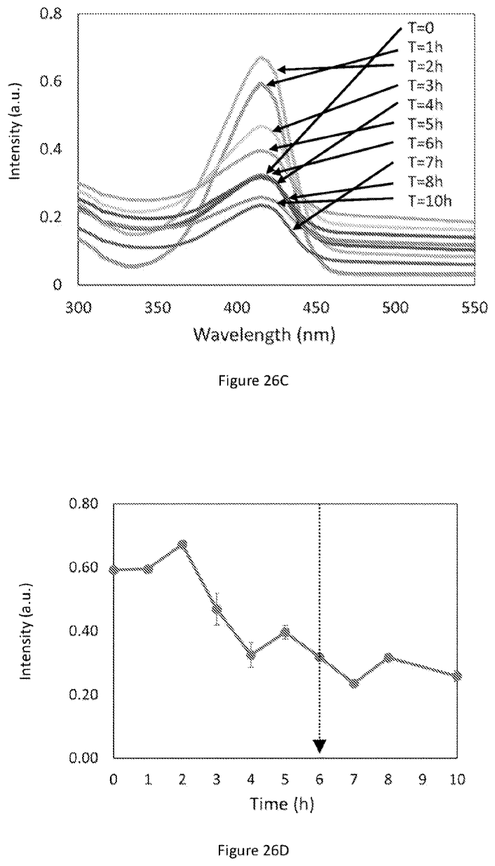

PLE enzyme was then added at t=6 h to determine whether the addition of this enzyme would affect the aggregation of Ester-containing TBA into hydrogel during the mesophase transition. HPLC analyses did not show a significant formation of cleaved tri-block, even after the addition of the PLE enzyme, which is responsive to the esterase end-group. The analysis of the formed Ester-TBA-based hydrogel revealed the presence of Ester-TBA and hydrolyzed tri-block in very small amounts (6%). These combined results indicate that the presence of the esterase enzyme does not affect the transition of polymeric hybrids from a micellar form to a hydrogel. The sharp decrease in the absorption within the initial 4 h (FIGS. 26A-26D) also aligns with the results obtained from the HPLC analysis.

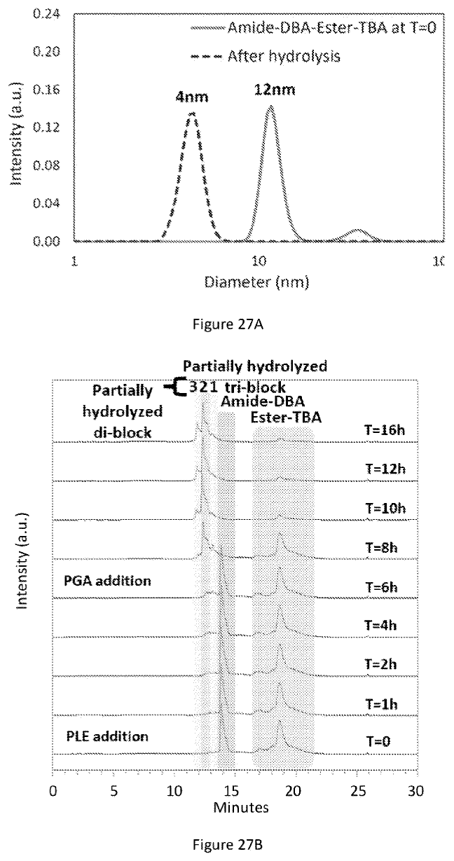

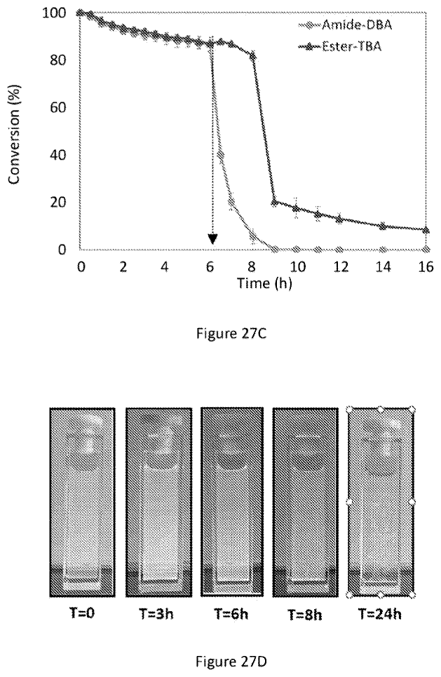

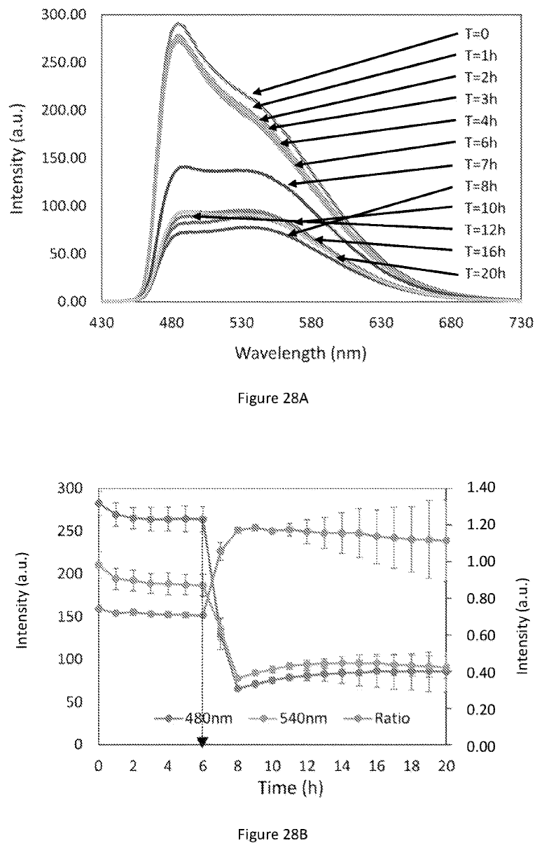

To better explore the features of the formulation in the presence of enzymes, an altered sequence of enzyme addition was performed whereby PLE was added first (0.28 μM or 10 U/mL) followed by the addition of PGA (0.96 μM or 0.525 U/mL). While DLS analysis showed a comparable result to the previous sequence (FIG. 27A), HPLC analysis revealed the slow degradation of both Amide-DBA and Ester-TBA (~18%) within 6 h of incubation with PLE (FIGS. 27B and 27C). However, at t=6 h, upon introducing PGA to the same solution, the concentration of the Amide-DBA dropped to 40% within 1 h and continued to decrease rapidly. At t=8 h, when nearly all the Amide-DBA had undergone degradation, the concentration of the Ester-TBA decreased to 20%, signifying its aggregation into a hydrogel state. This is also evident from the images taken at different time points as well as from the fluorescence results (FIG. 27D and FIGS. 28A-28B).

In order to determine the composition of the degraded gels, a small sample from each hydrolyzed polymer solution was extracted and analyzed by HPLC following a dilution with acetonitrile. The results of the HPLC analysis of a gel formed with the addition sequence of PGA at t=0 followed by PLE at t=6 h are shown in FIG. 29A and the results of the HPLC analysis of a gel formed with the addition sequence of PLE at t=0 followed by PGA at t=6 h are shown in FIG. 29B.

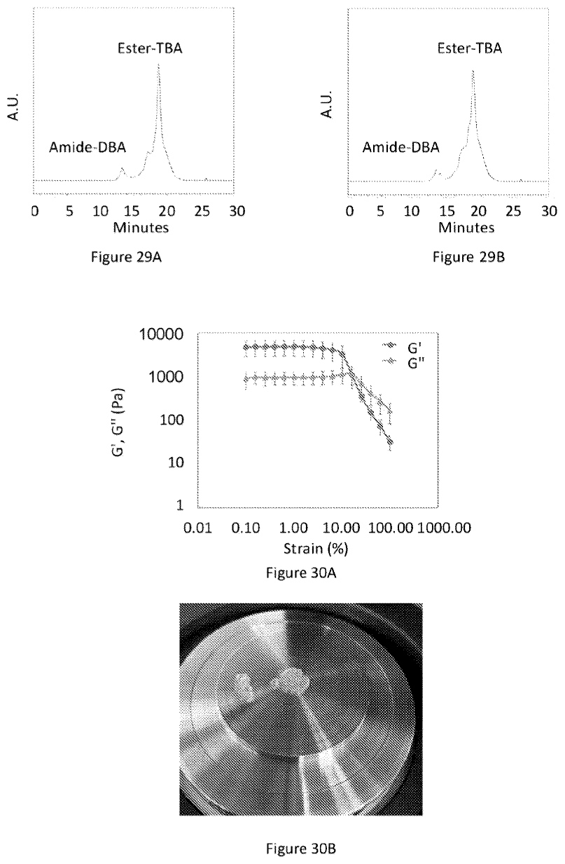

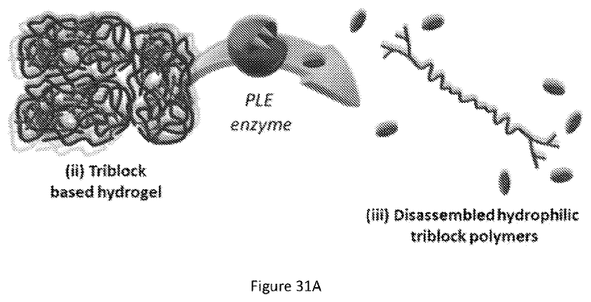

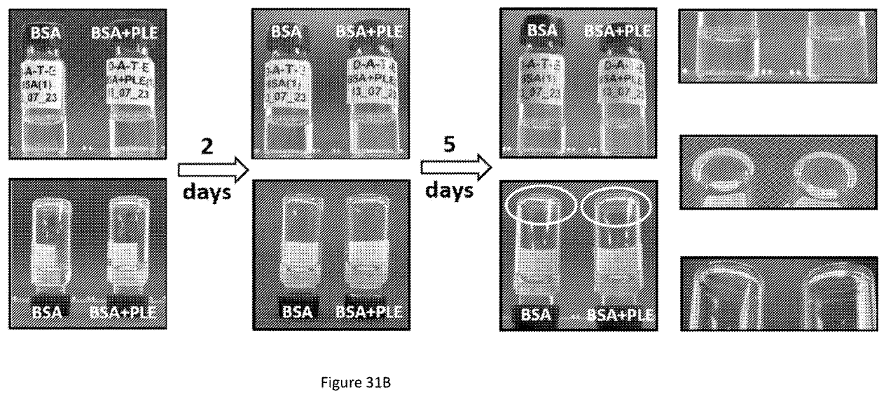

Rheological measurements were performed to characterize the Ester-TBA gel. The amplitude sweep test confirmed the gel-like behavior. In the linear viscoelastic region (LVE) of this sample, the elastic modulus G′ was higher than the viscous modulus G″ and it exhibited a distinct maximum at a higher strain value (FIG. 30A). The appearance of the hydrogel after conducting the rheology measurements was different than the appearance of the Amide-TBA (FIG. 30B vs. FIGS. 20A and 20B). Without being bound by any theory or mechanism of action, these differences are attributed to the relatively higher hydrophilicity of amide bonds as compared to the ester bonds.

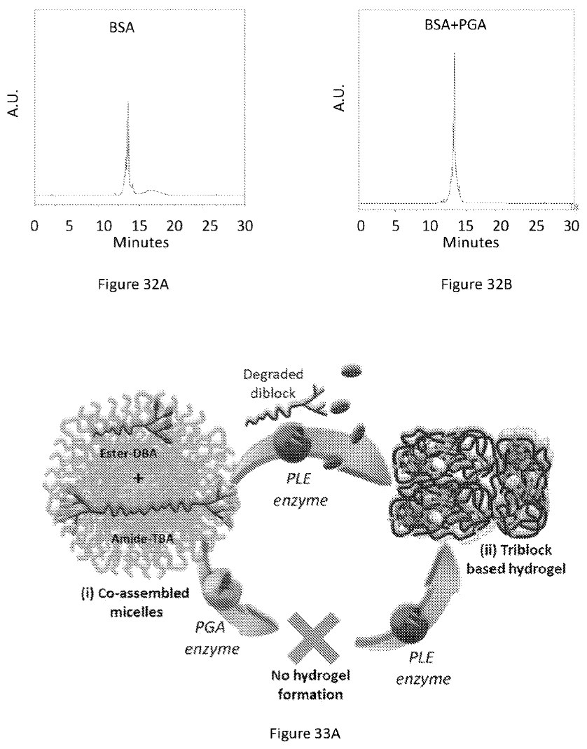

The hydrogel was further studied to determine its ability to undergo further enzymatic degradation and transform into soluble hydrophilic tri-block polymers (FIG. 31A). PLE was introduced with or without BSA. Images of the vials show the stability of the hydrogel in the presence of BSA and its full degradation into soluble hydrolyzed polymers in the presence of PLE (FIG. 31B). The HPLC results of a 100 μL solution obtained from the top layer of a hydrolyzed sample following 7 days of degradation and dilution with 100 μL of acetonitrile are shown in FIGS. 32A-32B.

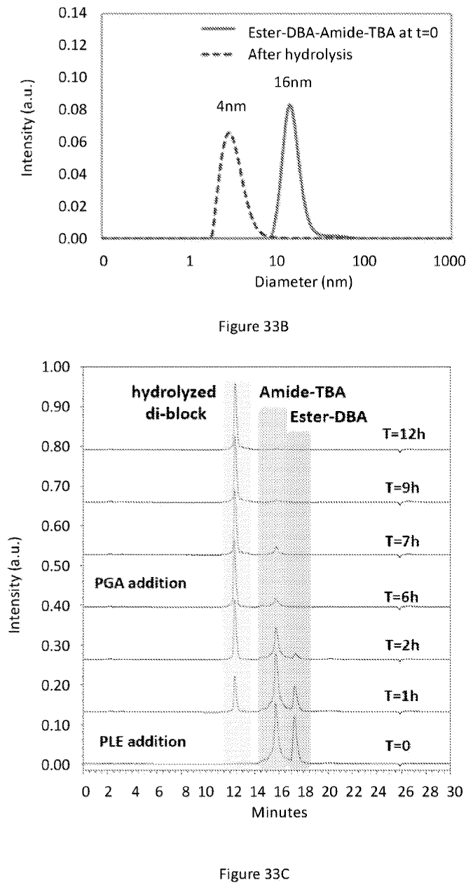

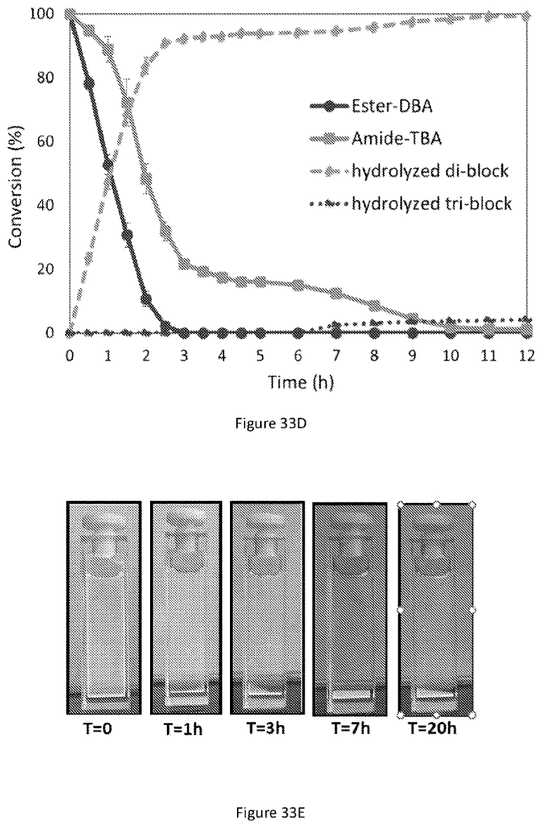

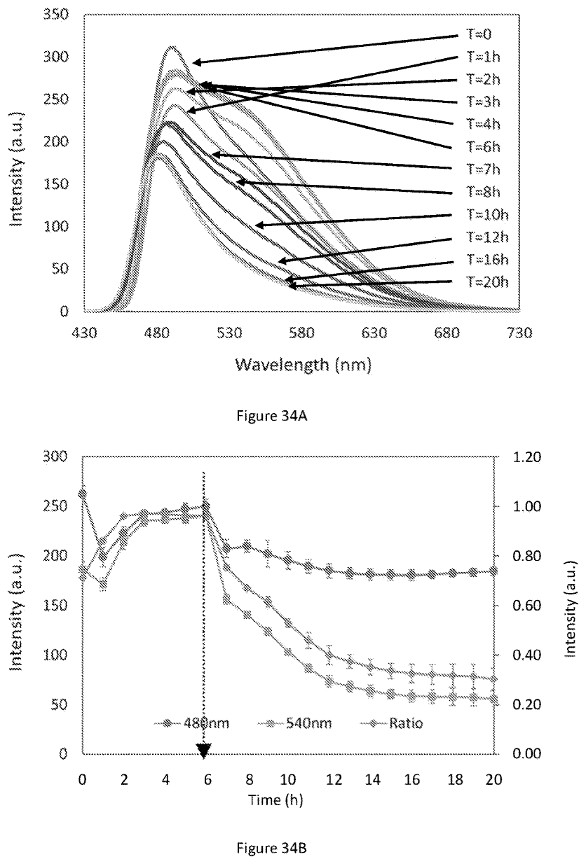

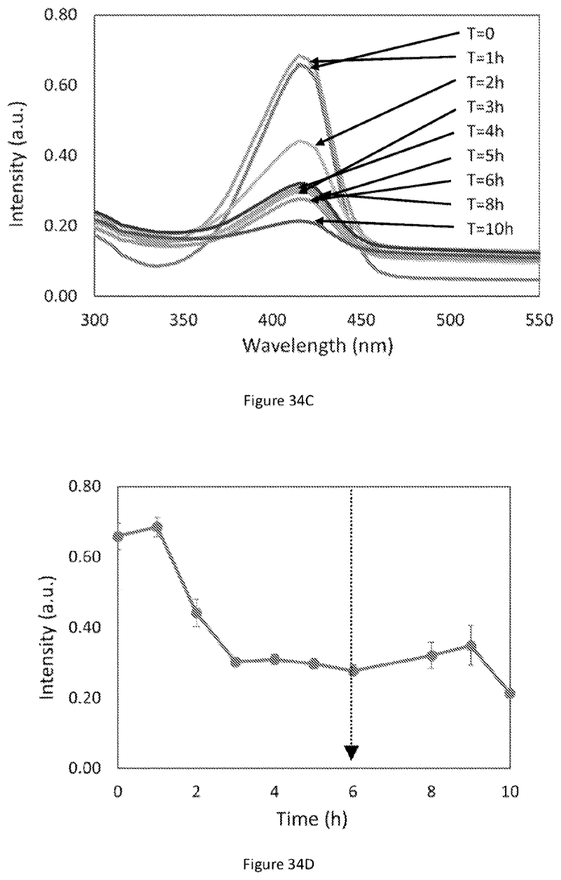

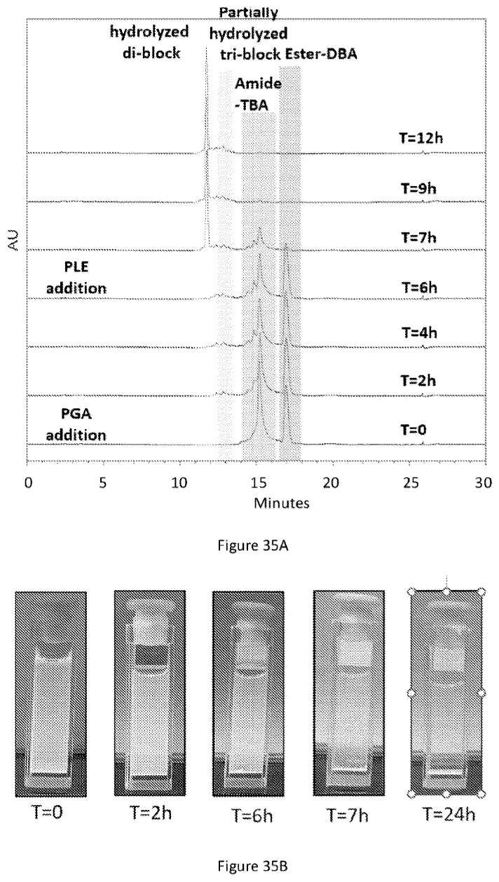

In a similar manner, a formulation formed by the combination of Ester-DBA and Amide-TBA at a 1:1 ratio was studied. The enzymatic degradation of this formulation was also monitored through two distinct pathways, as depicted schematically in FIG. 33A. The micellar solution was characterized by DLS (FIG. 33B) and initially incubated with the PLE enzyme. HPLC analysis demonstrated the high selectivity of the enzyme for the degradation of the Ester-DBA, which reached nearly 70% after 1.5 h. Next, a sudden drop of approximately 25% in the concentration of the Amide-TBA peak at t=2 h was observed (FIGS. 33C-33D). The chromatograms did not indicate a further significant simultaneous formation of cleaved tri-block structures (FIG. 33C), suggesting that the reduction in the Amide-TBA is not a result of its enzymatic degradation. Instead, it was attributed to its aggregation into a hydrogel within 1 h, as depicted by the images taken at different time points (FIG. 33E). A sharp decrease in the absorption within the initial 2 h (FIGS. 34A-34D) also aligns with the results obtained from the HPLC analyses. The addition of PGA at t=6 h did not yield any significant change except for the emergence of a small amount of cleaved tri-block (~5%).

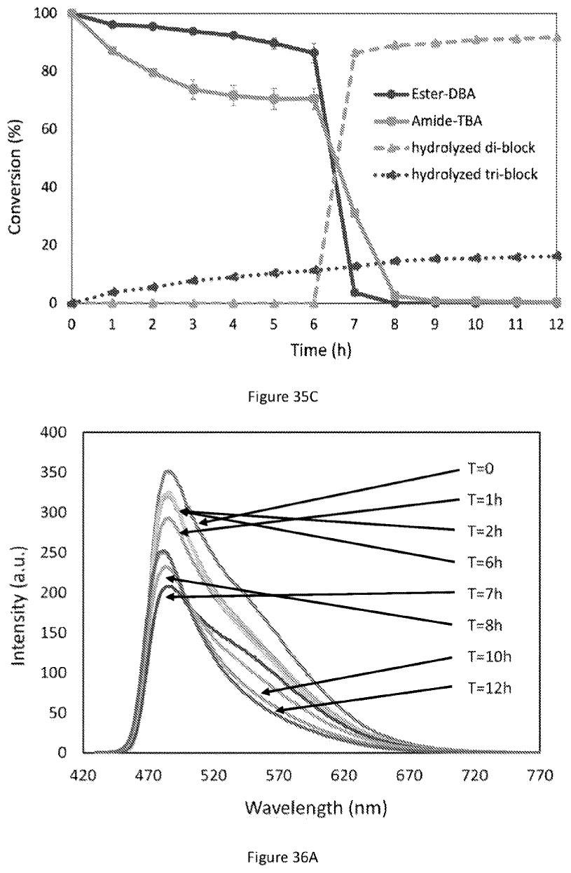

When the sequence of enzymes was altered to initially introduce PGA at t=0 followed by PLE at t=6 h, an even faster degradation of the Amide-TBA (~30%) compared to the Ester-DBA (~10%) during the first 6 h was observed. However, once PLE was added at t=6 h, a sharp decrease both in the concentration of the Ester-DBA and the Amide-TBA and simultaneous formation of hydrolyzed di-block were observed (FIGS. 35A-35C). The images taken at different time points (FIG. 35B) show a distinct gel aggregation process after incubating the sample with the PLE enzyme. This is also evident from the fluorescence results (FIGS. 36A-36B).

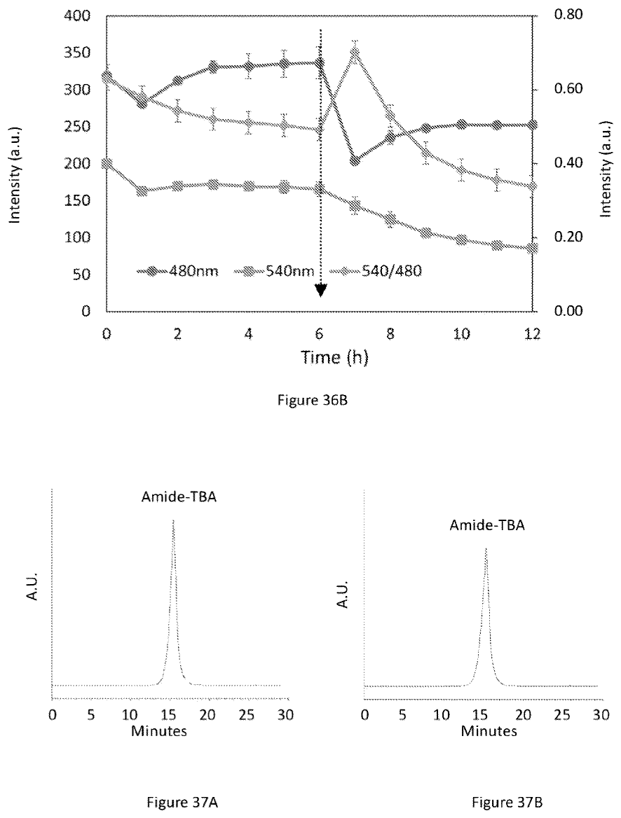

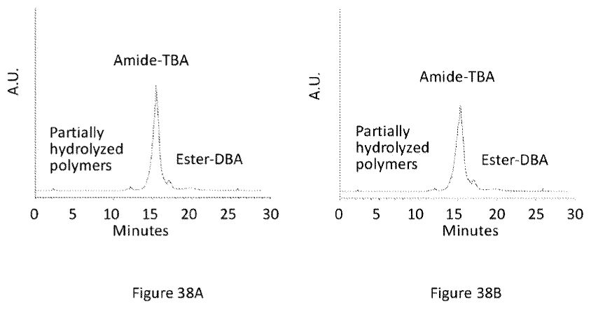

In order to determine the composition of the degraded gels, a small sample from each hydrolyzed polymer solution was extracted and analyzed by HPLC following a dilution with acetonitrile. The results of the HPLC analyses of a gel formed with the addition sequence of PLE at t=0 followed by PGA at t=6 h are shown in FIGS. 37A and 38A, and the results of the HPLC analyses of a gel formed with the addition sequence of PGA at t=0 followed by PLE at t=6 h are shown in FIGS. 37B and 38B.

Taken together, these results show the diversity of the formulations composed of di-block and tri-block amphiphiles according to embodiments of the present invention and their ability to successfully undergo multiple mesophase transitions when incubated with different enzymes. These transitions could be finely tuned by adjusting the sequence of enzyme addition. The use of more than one enzyme-responsive group in these formulations advantageously enables the tailoring of enzymatic selectivity and governing of the mechanical attributes of the resulting hydrogels.

### Example 7

### The Kinetics of Hydrogel Formation

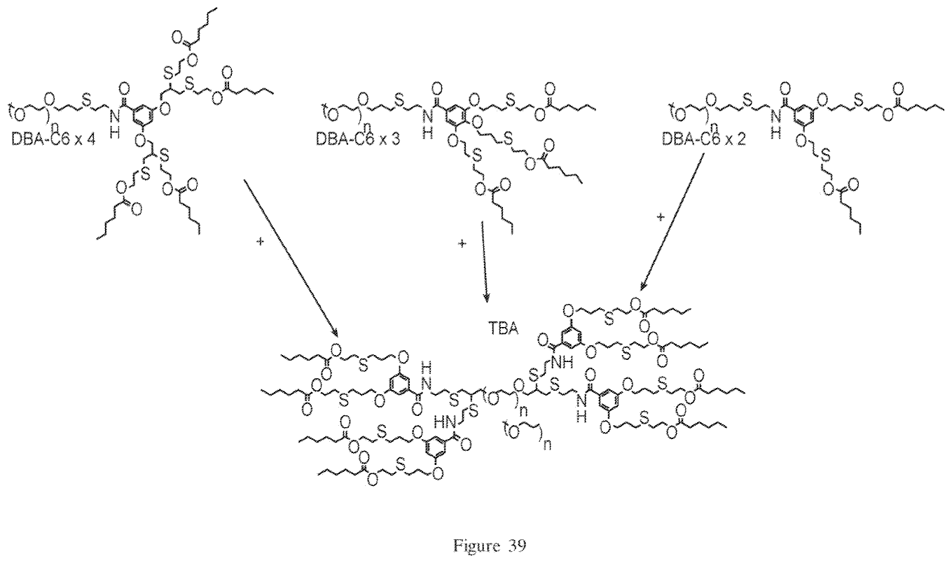

The structural features of the DBA hybrids and their ability to control the kinetics of hydrogel formation were studied. In particular, the number of end-groups of the DBA was changed and the mesophase transition upon exposure of a formulation incorporating the DBA hybrids to an activating enzyme was monitored. Three DBA hybrids were synthesized and combined with a TBA hybrid at a 2:1 ratio (FIG. 39).

The synthesis route for the preparation of the di-block amphiphile mPEG_{5kDa}-C6×2 (DBA-C6×2) is shown in Scheme 8.

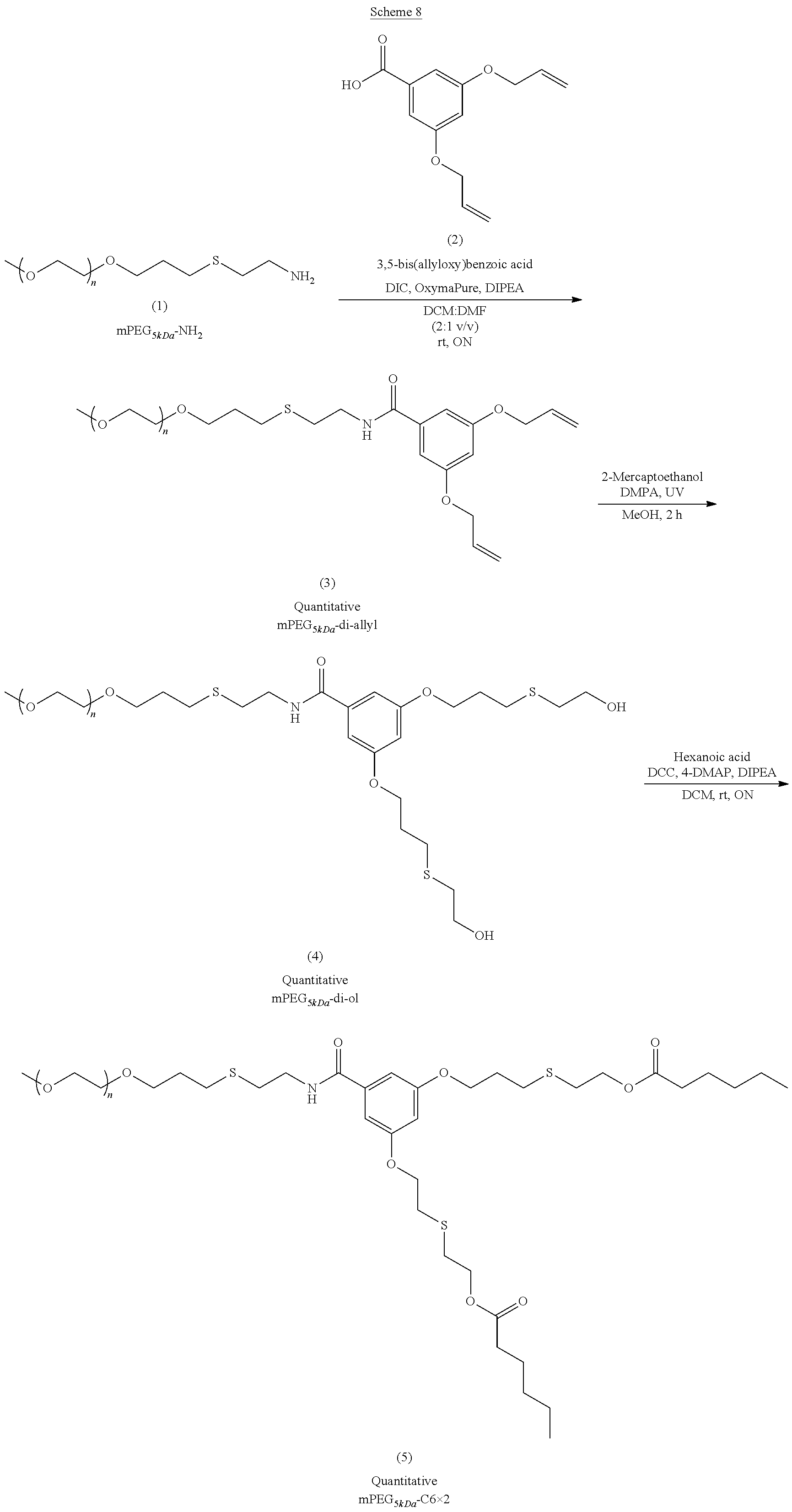

mPEG_{5kDa}-NH₂ was synthesized as reported in Buzhor et al., (Chem. Eur. J. 2015, 21, 15633-15638), the contents of which are hereby incorporated herein in their entirety. 3,5-bis(allyloxy)benzoic acid was synthesized as reported in Harnoy et al., (Synlett 2018; 29(19): 2582-2587), the contents of which are hereby incorporated herein in their entirety. 500 mg (0.096 mmol) of mPEG_{5kDa}-NH₂ and 112.7 mg (0.48 mmol) of 3,5-bis(allyloxy)benzoic acid were dissolved in DCM:DMF (2:1 v/v, 2 mL). OxymaPure (68.41 mg, 0.48 mmol), DIPEA (124 mg, 0.96 mmol), and DIC (60.75 mg, 0.48 mmol) were added to the solution and the solution was stirred at room temperature overnight. The crude mixture was then loaded on a MeOH-based LH20 SEC column. The fractions that contained the product were unified, and MeOH was evaporated in vacuum. The product, mPEG_{5kDa}-di-allyl, was obtained as a pale pinkish-white solid (quantitative yield).

mPEG_{5kDa}-di-allyl (500 mg, 0.092 mmol) and 2-mercapatoehtnaol (361 mg, 4.62 mmol) were dissolved in MeOH (3 mL). DMPA (11.84 mg, 0.046 mmol) was added to the solution and the solution was purged with nitrogen for 15 minutes and then placed under UV light at 365 nm for 2 hours. The crude mixture was loaded on a MeOH-based LH20 SEC column. The fractions that contained the product were unified, and MeOH was evaporated in vacuum. The product, mPEG_{5kDa}-di-ol, was obtained as an off-white solid (quantitative yield).

mPEG_{5kDa}-di-ol (500 mg, 0.089 mmol) and hexanoic acid (104.4 mg, 0.89 mmol) were dissolved in DCM (10 mL). DCC (185 mg, 0.898 mmol) and DMAP (54.8 mg, 0.45 nmol) were added to the solution and the solution was stirred at room temperature overnight. The crude product was filtered with a 0.22 μm PTFE syringe filter and loaded on a MeOH-based LH20 SEC column. The fractions that contained the product were unified, and MeOH was evaporated in vacuum. The product, mPEG_{5kDa}-C6×2, was obtained as an off-white solid (quantitative yield) and characterized by NMR.

The synthesis route for the preparation of the di-block amphiphile mPEG_{5kDa}-C6×3 (DBA-C6×3) is shown in Scheme 9.

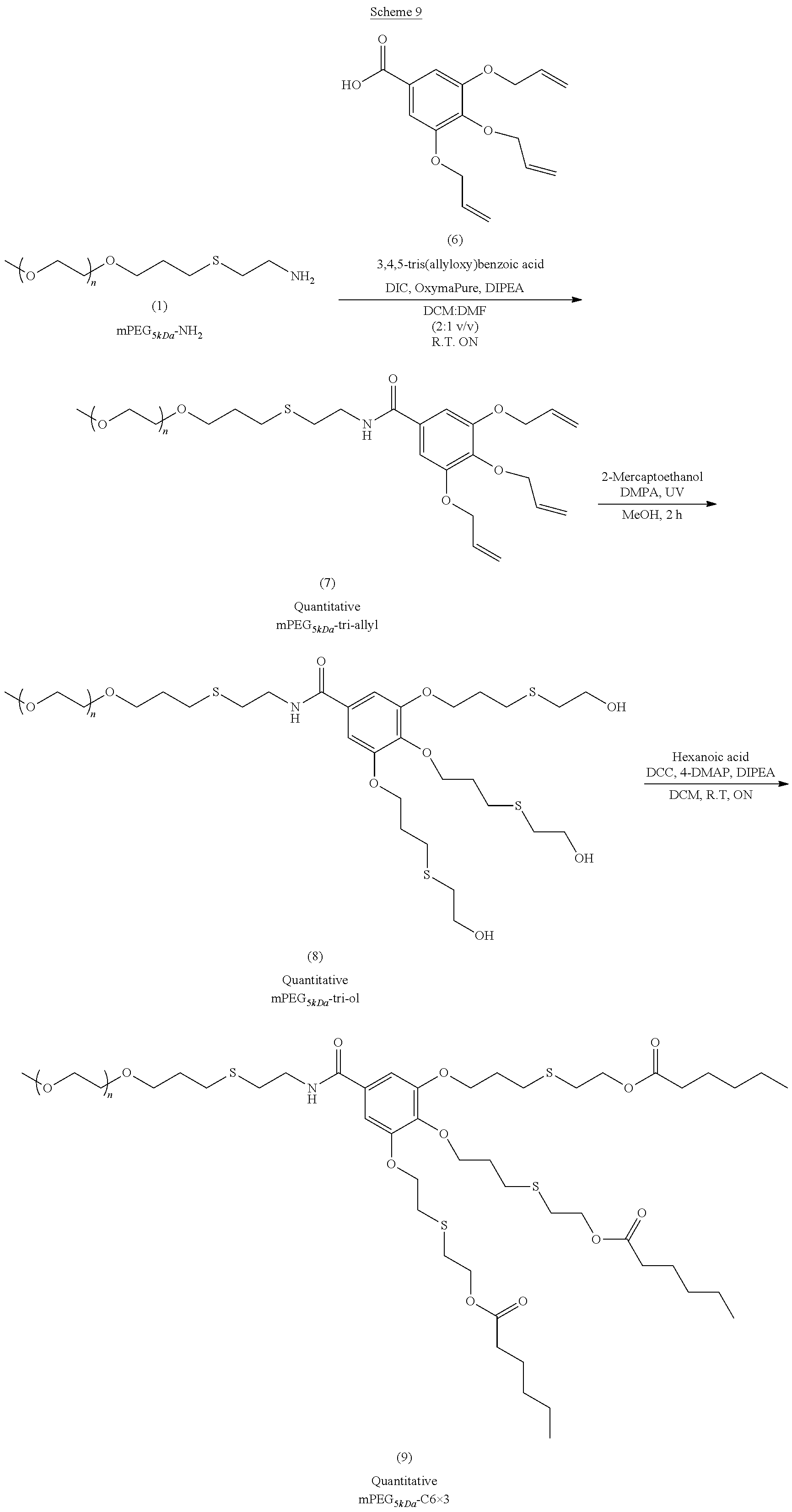

3,5-tris(allyloxy)benzoic acid was synthesized as reported in Harnoy et al., (Biomacromolecules 2017, 18, 4, 1218-1228), the contents of which are hereby incorporated herein in their entirety. mPEG_{5kDa}-NH₂ (500 mg, 0.096 mmol) and 3,5-tris(allyloxy)benzoic acid (139.76 mg, 0.48 mmol) were dissolved in DCM:DMF (2:1 v/v, 3 mL). OxymaPure (68.41 mg, 0.48 mmol), DIPEA (124 mg, 0.96 mmol), and DIC (60.75 mg, 0.48 mmol) were added to the solution and the solution was stirred at room temperature overnight. The crude mixture was then loaded on a MeOH-based LH20 SEC column. The fractions that contained the product were unified, and MeOH was evaporated in vacuum. The product, mPEG_{5kDa}-tri-allyl, was obtained as a pale pinkish-white solid (quantitative yield).

mPEG_{5kDa}-tri-allyl (500 mg, 0.091 mmol) and 2-mercapatoehtnaol (2.14 g, 27.44 mmol) were dissolved in MeOH (2 mL). DMPA (23.44 mg, 0.091 mmol) was added to the solution and the solution was purged with nitrogen for 15 minutes and then placed under UV light at 365 nm for 2 hours. The crude mixture was loaded on a MeOH-based LH20 SEC column. The fractions that contained the product were unified, and MeOH was evaporated in vacuum. The product, mPEG_{5kDa}-tri-ol, was obtained as an off-white solid (quantitative yield).

mPEG_{5kDa}-tri-ol (200 mg, 0.035 mmol) and hexanoic acid (61 mg, 0.052 mmol) were dissolved in DCM (10 mL). DCC (108.61 mg, 0.052 mmol) and DMAP (21.43 mg, 0.175 mmol) were added to the solution and the solution was stirred at room temperature overnight. The crude product was then filtered with a 0.22 μm PTFE syringe filter and loaded on a MeOH-based LH20 SEC column. The fractions that contained the product were unified, and MeOH was evaporated in vacuum. The product, mPEG_{5kDa}-C6×3, was obtained as an off-white solid (quantitative yield) and characterized by NMR.

The synthesis route for the preparation of the di-block amphiphile mPEG_{5kDa}-C6×4 (DBA-C6×4) is shown in Scheme 10.

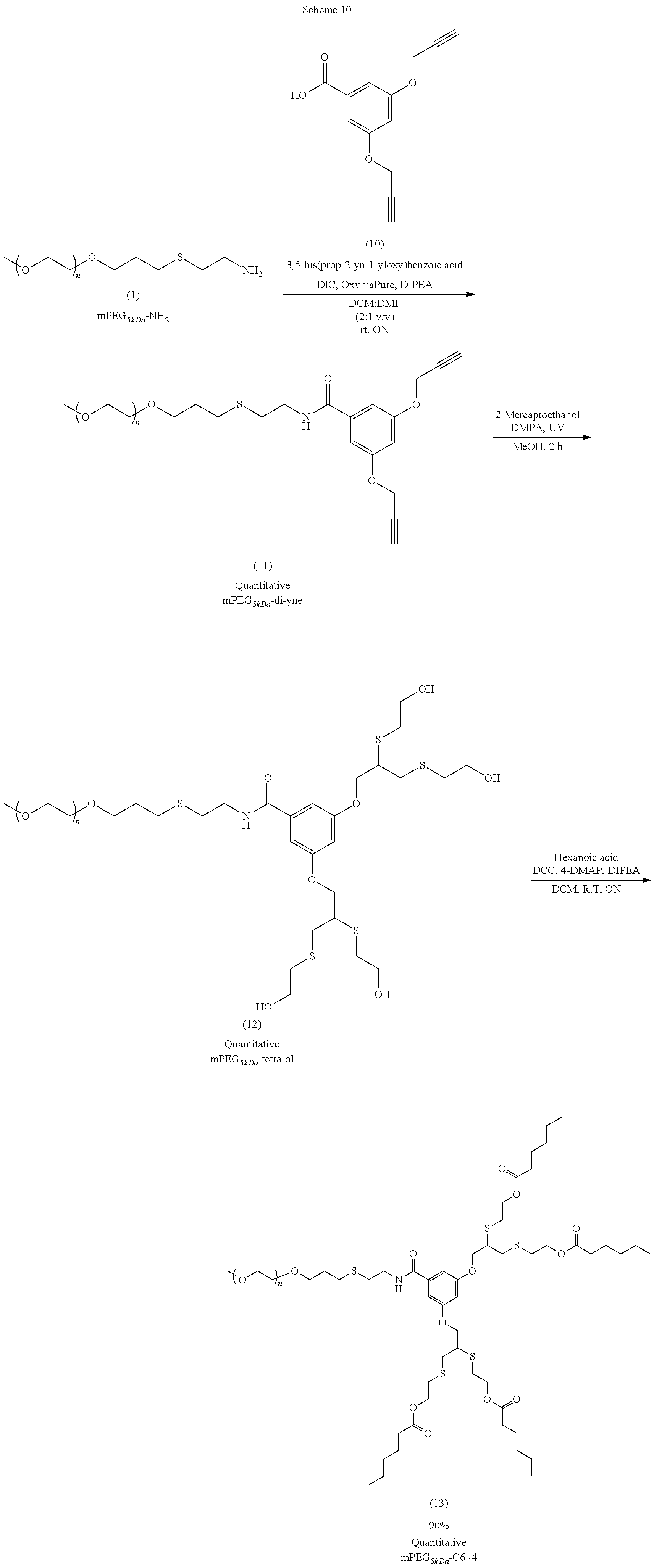

3,5-bis(prop-2-yn-1-yloxy)benzoic acid was synthesized as reported in Harnoy et al., (Biomacromolecules 2017, 18, 4, 1218-1228), the contents of which are hereby incorporated herein in their entirety. mPEG_{5kDa}-NH₂ (500 mg, 0.096 mmol) and 3,5-bis(prop-2-yn-1-yloxy)benzoic acid (110.83 mg, 0.48 mmol) were dissolved in DCM:DMF (2:1 v/v, 3 mL). OxymaPure (68.41 mg, 0.48 mmol), DIPEA (124 mg, 0.96 mmol), and DIC (60.75 mg, 0.48 mmol) were added to the solution and the solution was stirred at room temperature overnight. The crude mixture was loaded on a MeOH-based LH20 SEC column. The fractions that contained the product were unified, and MeOH was evaporated in vacuum. The product, mPEG_{5kDa}-di-yne, was obtained as a pale off-white solid (quantitative yield).

mPEG_{5kDa}-di-yne (500 mg, 0.092 mmol) and 2-mercapatoehtnaol (2.16 g, 27.75 mmol) were dissolved in MeOH (3 mL). DMPA (23.7 mg, 0.092 mmol) was added to the solution and the solution was purged with nitrogen for 15 minutes and then placed under UV light at 365 nm for 2 hours. The crude mixture was loaded on a MeOH-based LH20 SEC column. The fractions that contained the product were unified, and MeOH was evaporated in vacuum. The product, mPEG_{5kDa}-tetra-ol, was obtained as an off-white solid (quantitative yield).

mPEG_{5kDa}-tetra-ol (400 mg, 0.089 mmol) and hexanoic acid (162.51 mg, 1.399 mmol) were dissolved in DCM (10 mL). DCC (288.67 mg, 1.399 mmol) and DMAP (42.73 mg, 0.349 mmol) were added to the solution and the solution was stirred at room temperature overnight. The crude product was filtered with a 0.22 μm PTFE syringe filter and loaded on a MeOH-based LH20 SEC column. The fractions that contained the product were unified, and MeOH was evaporated in vacuum. The product, mPEG_{5kDa}-C6×4-compound, was obtained as an off-white solid (90% yield) and characterized by NMR.

The synthesis route for the preparation of the tri-block amphiphile PEG_{10kDa}-C6×8 is shown in Scheme 11.

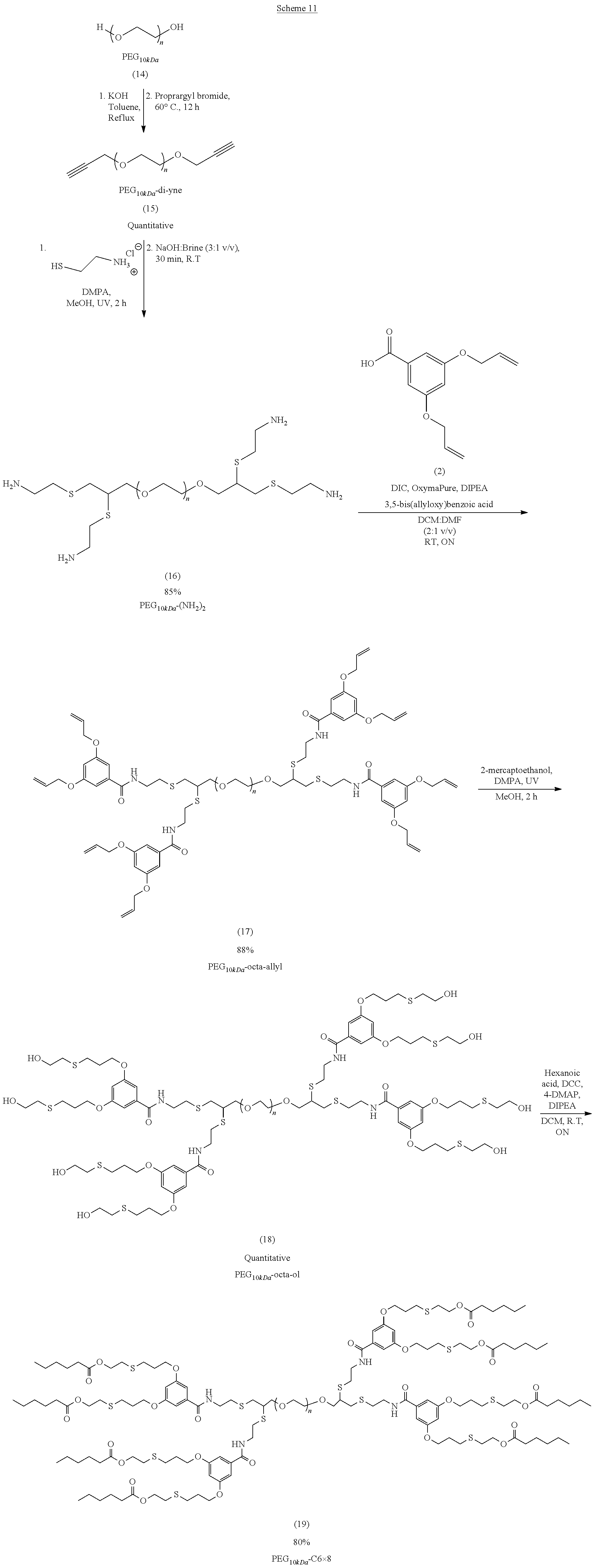

PEG_{10kDa} (10 g, 1 mmol) was dissolved in toluene (50 mL) in a round-bottom (RB) flask, along with KOH (3.33 g, 60 mmol), and refluxed at 140° C. (using a Dean-Stark apparatus) for 2 hours. The temperature was reduced to 60° C. and the Dean-Stark apparatus was removed. Propargyl bromide (6.96 g, 60 mmol) was slowly added to the RB flask, and the solution was left to stir at 60° C. for 12 hours. The solution was then hot-filtered through celite, and the celite was washed with DCM (3×30 mL). The combined organic solvents were evaporated, and the residue was resuspended in DCM (5-10 mL). PEG_{10kDa}-di-yne was precipitated with ether (200 mL), and the precipitate was filtered and washed with ether (3×15 mL). PEG_{10kDa}-di-yne was then dried under high vacuum for 12 hours and obtained as a white solid in quantitative yield.

PEG_{10kDa}-di-yne (900 mg, 0.09 mmol), cysteamine hydrochloride (2.04 g, 19 mmol), and DMPA (23.04 mg, 0.09 mmol) were dissolved in 3 mL of MeOH in an RB flask along with a magnetic stirrer and purged with nitrogen for 15 minutes. Then, the RB flask was stirred under UV light at 365 nm for 2 hours. The reaction was quenched by allowing air into the RB flask for 2 minutes. The solution was then directly transferred into a MeOH-based LH20 SEC column. The fractions that contained the product were collected, and MeOH was evaporated under vacuum. The product, PEG_{10kDa}-(NH₂)₂, was obtained as an off-white solid (85% yield).

PEG_{10kDa}-(NH₂)₂ (1.2 g, 0.11 mmol) and 3,5-bis(allyloxy)benzoic acid (269.12 mg, 1.14 mmol) were dissolved in DCM:DMF (2:1 v/v, 3 mL). OxymaPure (163.26 mg, 1.14 mmol), DIPEA (269 mg, 2.29 mmol), and DIC (144.98 mg, 1.14 mmol) were added to the solution and the solution was stirred at room temperature overnight. The crude mixture was then loaded on a MeOH-based LH20 SEC column. The fractions containing the product were combined, and MeOH was evaporated under vacuum. The product, PEG_{10kDa}-octa-allyl, was obtained as a pale pinkish-white solid (88% yield).

PEG_{10kDa}-Octa-allyl (850 mg, 0.076 mmol) and 2-mercaptoethanol (1.49 g, 19.09 mmol) were dissolved in MeOH (4 mL). DMPA (15.65 mg, 0.061 mmol) was added to the solution and the solution was purged with nitrogen for 15 minutes and then placed under UV light at 365 nm for 2 hours. The crude mixture was loaded on a MeOH-based LH20 SEC column. The fractions containing the product were combined, and MeOH was evaporated under vacuum. The product, PEG_{10kDa}-octa-ol, was obtained as an off-white solid (quantitative yield).

PEG_{10kDa}-octa-ol (690 mg, 0.057 mmol) and hexanoic acid (67.14 mg, 0.57 mmol) were dissolved in DCM (10 mL). DCC (119.27 mg, 0.578 mmol) and DMAP (35.31 mg, 0.289 mmol) were added to the solution and the solution was stirred at room temperature overnight. The crude mixture was then filtered with a 0.22 μm PTFE syringe filter and loaded onto a MeOH-based LH20 SEC column. The fractions containing the product were combined, and MeOH was evaporated under vacuum. The product, PEG_{10kDa}-C6×8, was obtained as an off-white solid (80% yield) and characterized by NMR.

The formation of hydrogels upon addition of PLE (0.5 U/mL) was studied using HPLC and absorption spectroscopy.

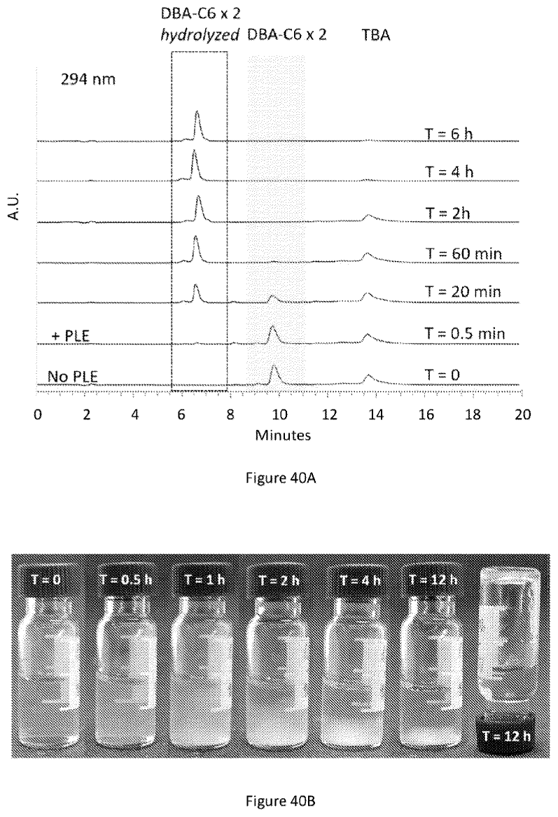

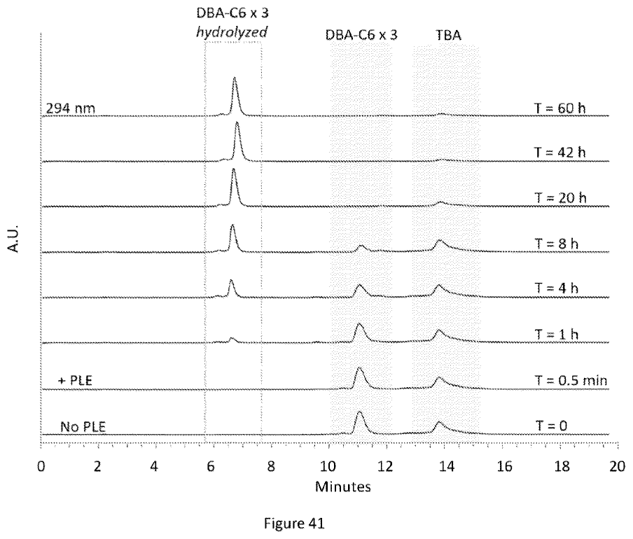

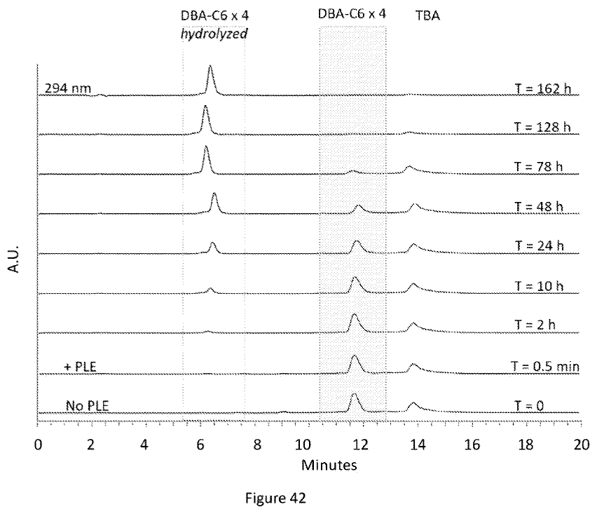

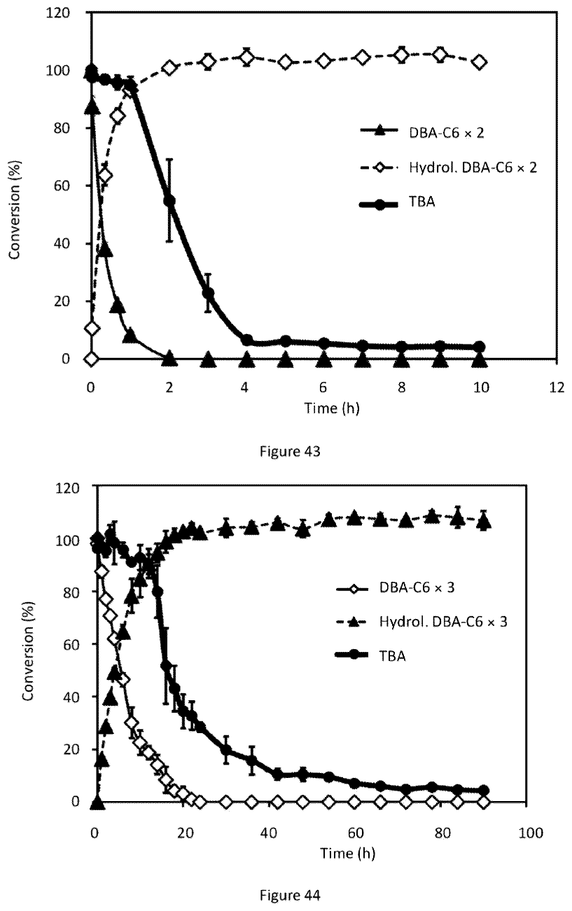

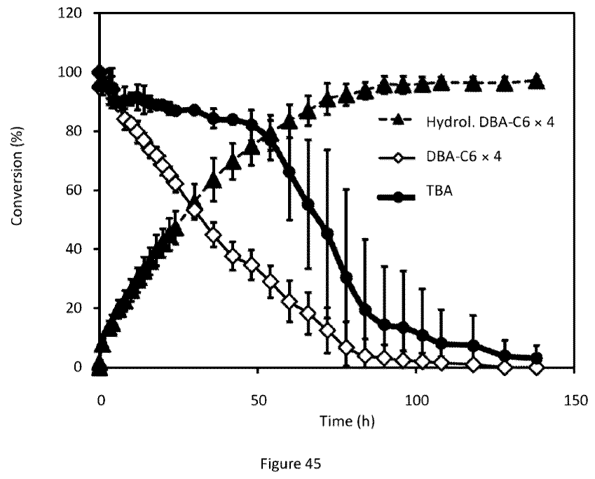

FIG. 40A shows the HPLC data of DBA-C6×2 and TBA over time in the presence of PLE indicating the precipitation of the TBA after complete DBA-C6×2 hydrolysis. This is also evident from the images taken at different time points (FIG. 40B). FIGS. 41 and 42 show the HPLC data of DBA-C6×3 and DBA-C6×4, respectively, and TBA in the presence of PLE. The kinetic data of the formulations with DBA-C6×2, DBA-C6×3, and DBA-C6×4 are shown in FIGS. 43, 44, and 45, respectively. The results indicate that an increase in the hydrophobicity of the DBA by increasing the number of hydrophobic dendrons induces a slower formation of the TBA-based hydrogel upon activation by a first enzyme.

### Example 8

### Enzymatic Degradation and Hydrogel Formation

Additional DBA hybrids exerting different degrees of hydrophobicity due to the different number of carbon atoms in the hydrophobic end groups were synthesized as depicted in Scheme 12.

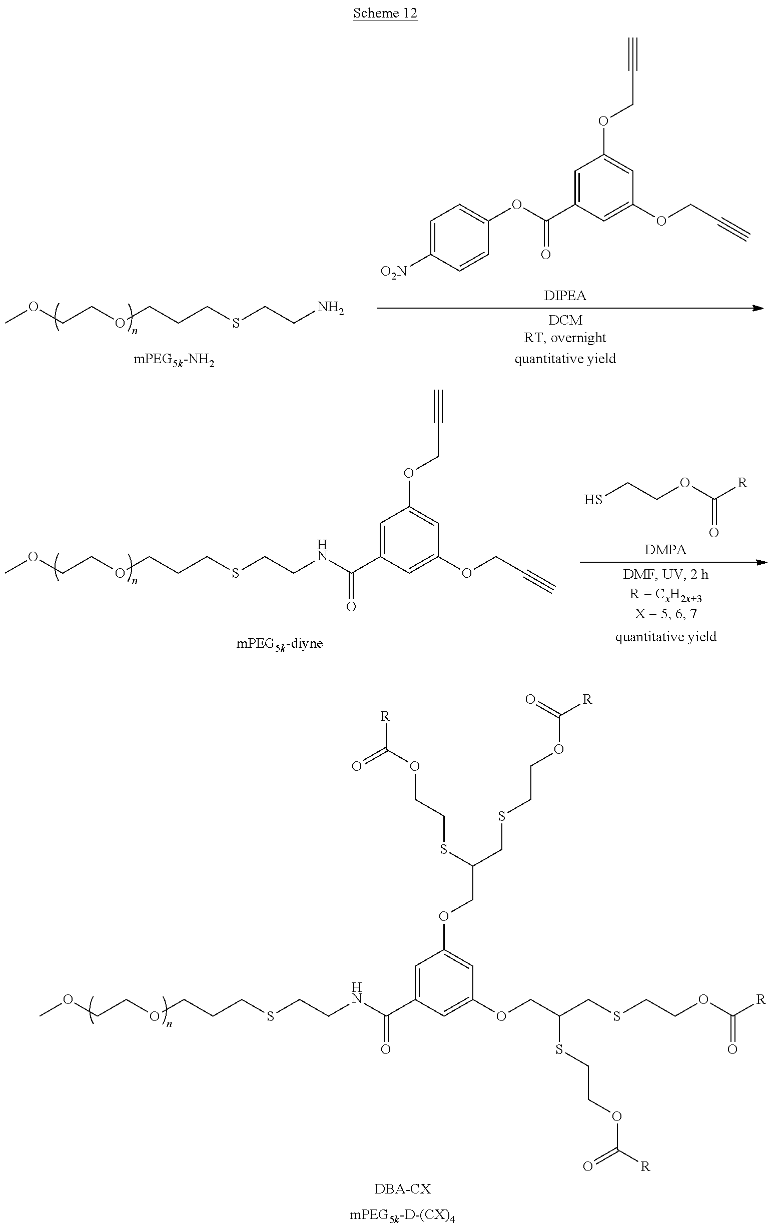

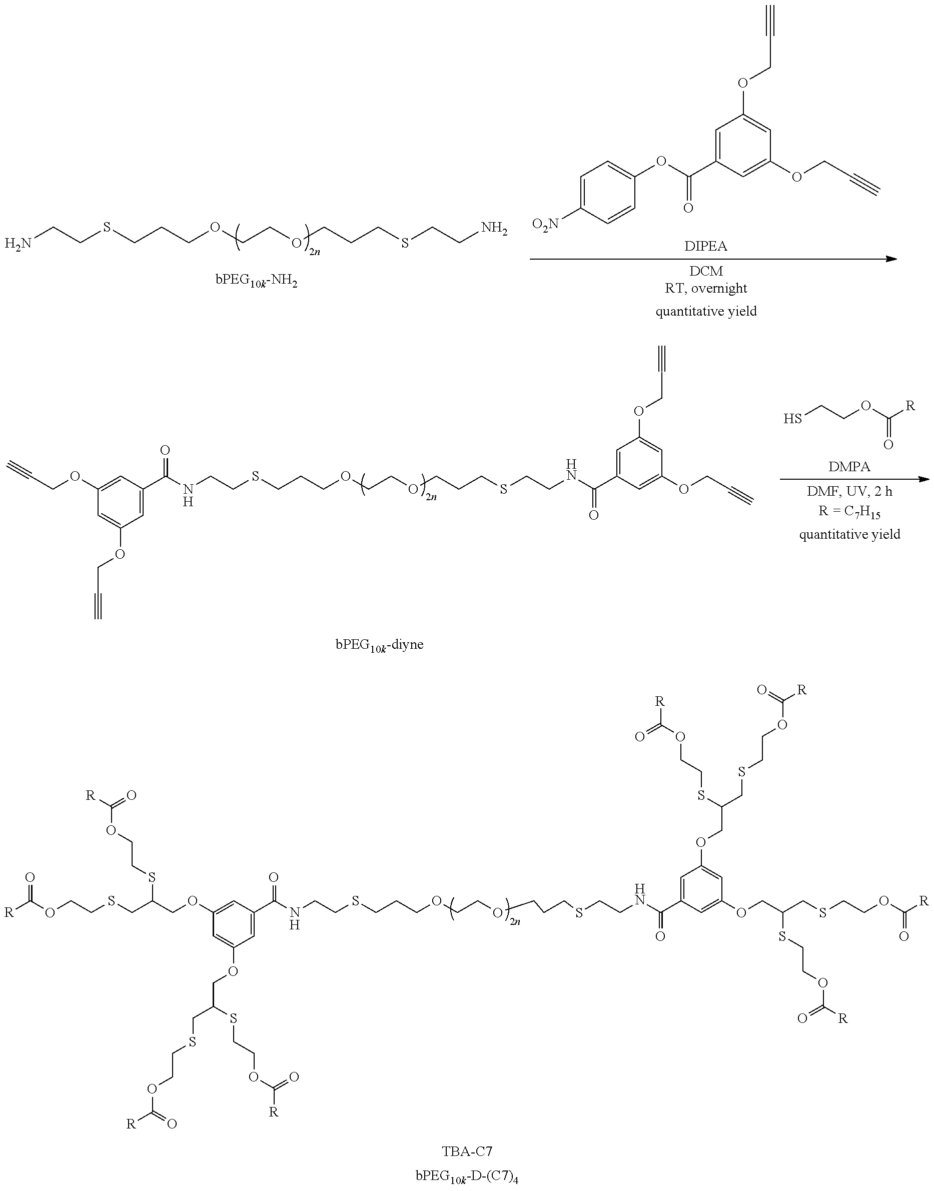

The DBA hybrids were termed as follows: mPEG₅ₖ-(D)-4×C5, mPEG₅ₖ-(D)-4×C6, and mPEG₅ₖ-(D)-4×C7. The DBA hybrids were mixed with a TBA hybrid bPEG₁₀ₖ-(D)-4×C7 at a 3:2 weight ratio followed by the addition of 0.7 μM PLE.

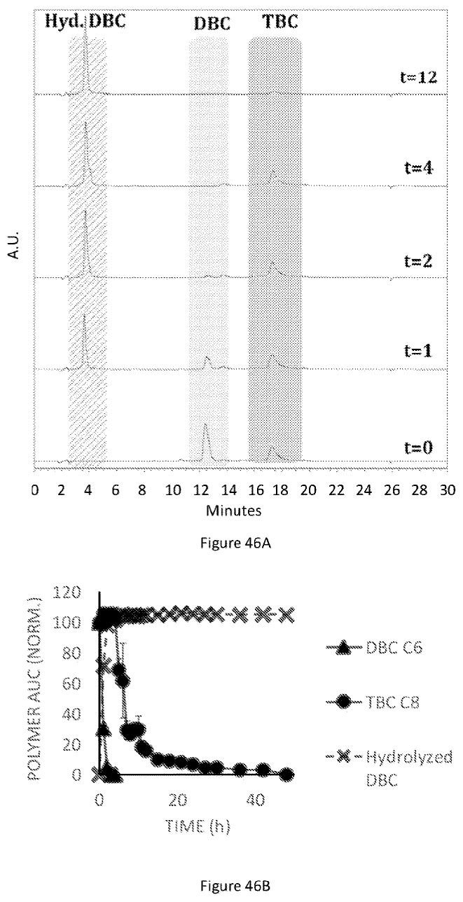

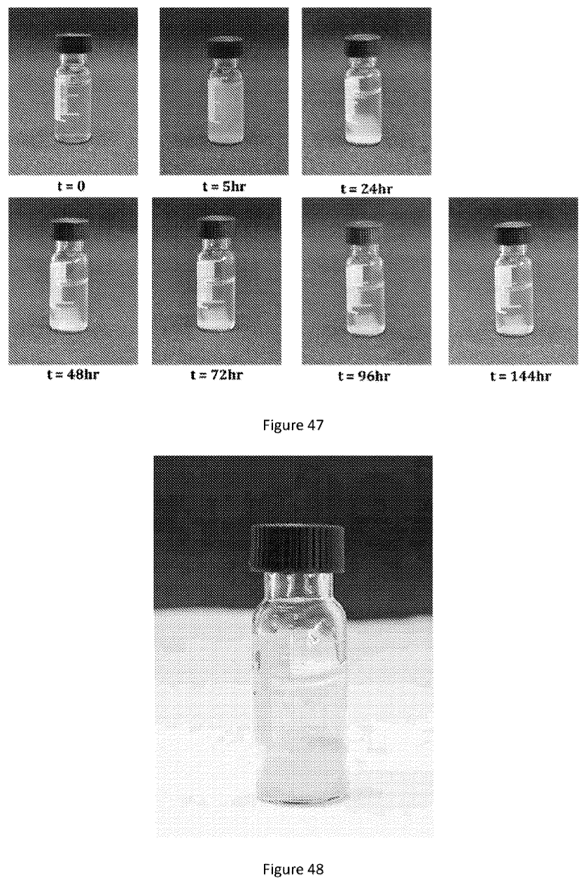

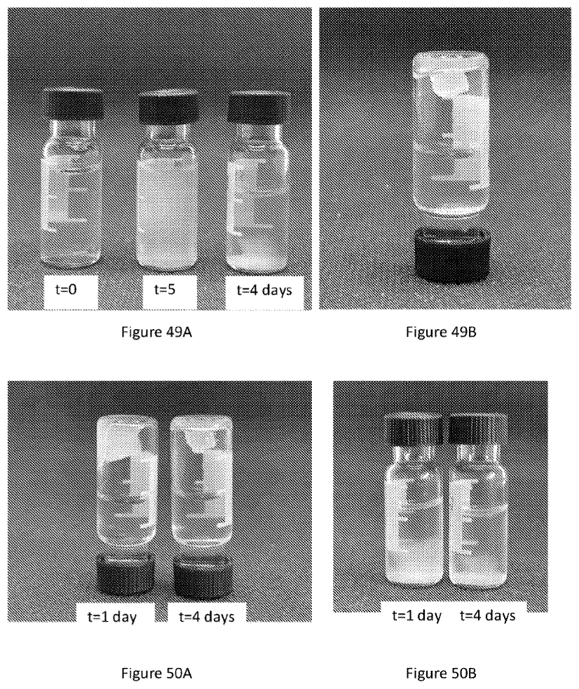

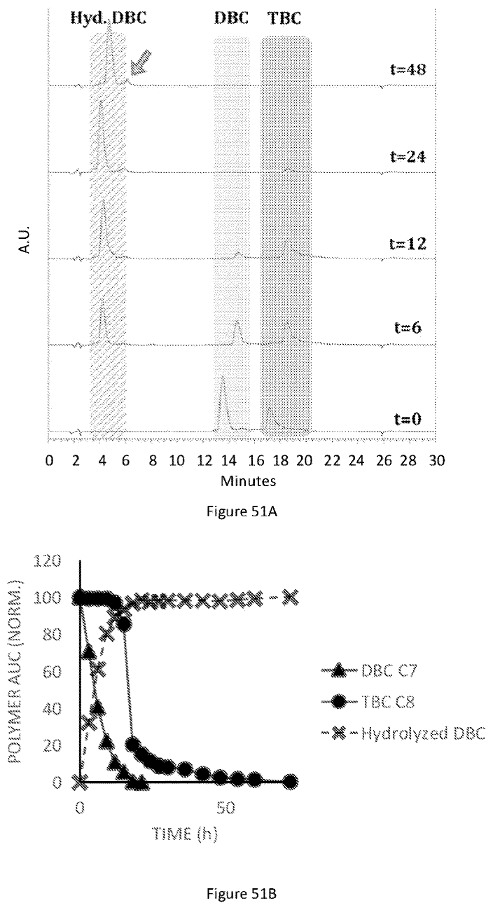

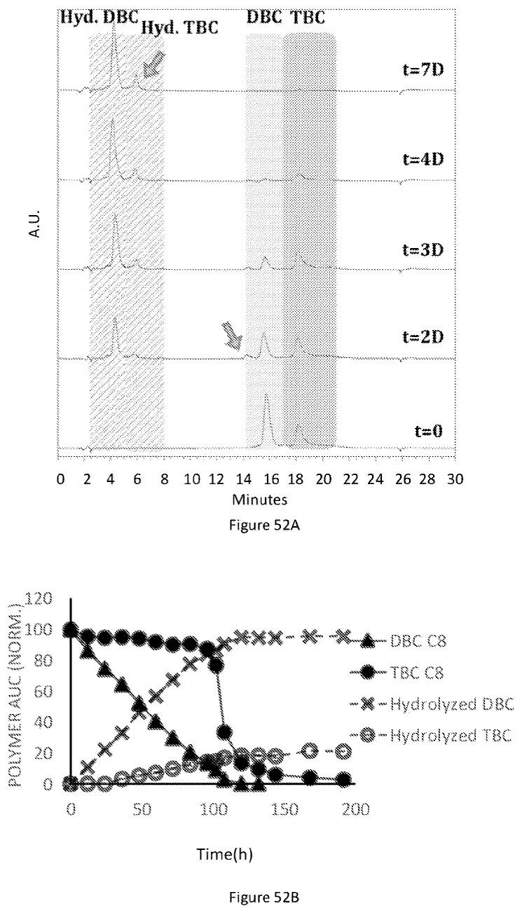

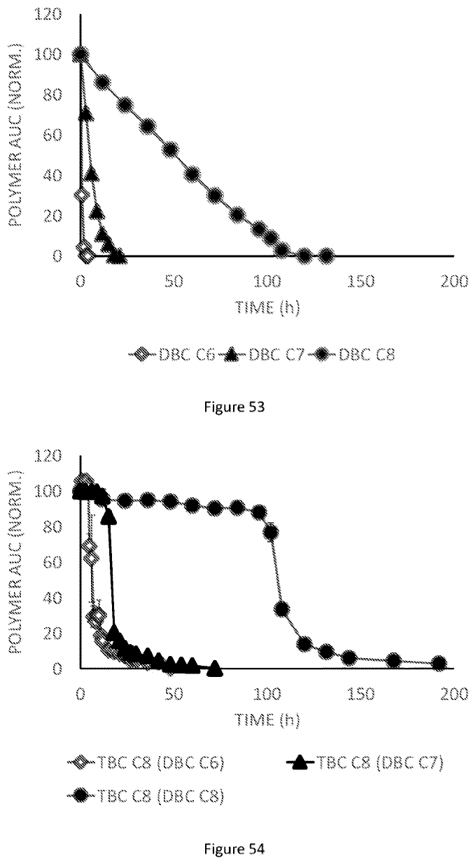

FIGS. 46A-46B show the HPLC data of DBA-4×C5 and TBA over time in the presence of PLE, indicating the precipitation of TBA only after complete hydrolysis of DBA. This is also evident from the images taken at different time points (FIGS. 47 and 48). Similar trends were observed also for DBA-4×C6 and DBA-4×C7 (FIGS. 51A-51B and 52A-52B, respectively). FIGS. 53 and 54 demonstrate the different kinetics for DBA degradation and TBA aggregation, respectively. In addition, a change in hydrogel properties over time (hydrogel “aging”) was observed, as shown in FIGS. 49A-49B and 50A-50B.

These results demonstrate that even a minor change in the hydrophobicity of the DBA has a significant impact on the kinetics of enzymatic degradation of these amphiphiles and, consequently, on the mesophase transition into a TBA-based hydrogel. Overall, these findings highlight the ability to use molecular architecture and hydrophobicity towards programming mesophase transitions of polymeric assemblies.

Taken together, the results show a variety of formulations composed of a mixture of di- and tri-block copolymers capable of undergoing mesophase transitions whose timing can be tailored. The formations are capable of maintaining a cargo encapsulated in micelles formed by the self-assembly of the di- and tri-block copolymers and afford its controlled release at a target site of action upon application of a stimulus or a plurality to stimuli. Hence, the present invention provides programmable mesophase shifting formulations by co-assembly of stimuli-responsive amphiphiles with different kinetic behaviors and functions.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1.-32. (canceled)

33. A delivery system, comprising:
a mixture of a di-block copolymer and a tri-block copolymer;
wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween;
wherein the delivery system is in the form of self-assembled micelles or fibers that transform to micelles upon hydration;
wherein the first stimulus-responsive cleavable site undergoes cleavage upon exposure to a first stimulus thereby invoking a first phase transition from micelles to a hydrogel, and the second stimulus-responsive cleavable sites undergo cleavage upon exposure to a second stimulus thereby disassembling the hydrogel and invoking a second phase transition to dissolved polymers and fragments thereof.

34. The delivery system of claim 33 which is in the form of self-assembled micelles.

35. The delivery system of claim 33, wherein the hydrophilic segment of the di- and/or tri-block copolymer comprises polyacrylic acid, poly(hydroxyethyl acrylate), polyethylene glycol (PEG), poly(oligo-ethylene glycol acrylate), polyacrylamide, polymethyl oxazoline, polyethyl oxazoline, polysarcosine, polypeptide, polypeptoid, hydrophilic polymethacrylate, polyamine, hydrophilic nylon, polyvinyl alcohol, hydrophilic protein, or polycarbohydrate.

36. The delivery system of claim 35, wherein the hydrophilic segment of the di- and/or tri-block copolymer comprises polyacrylic acid, poly(2-hydroxyethyl acrylate), polyethylene glycol (PEG), or poly(oligo-ethylene glycol acrylate).

37. The delivery system of claim 33, wherein the hydrophilic segment of the di- and/or tri-block copolymer is linked to the hydrophobic segment(s) by a group selected from the group consisting of —Z—X—, —X¹—Z—X²—, and —Z¹—X¹—Z²—X²—, wherein Z, Z¹, and Z² are each independently selected from C₁-C₁₀ alkylene, C₂-C₁₀ alkenylene, C₂-C₁₀ alkynylene, and arylene; X, X¹, and X² are each independently selected from —O—; —S—; —NH—; —C(═O)—; —C(═O)—O—; —O—C(═O)—O—; —C(═O)—NH—; —NH—C(═O)—NH—; —NH—C(═O)—O—; —S(═O)—; —S(═O)—O—; —PO(═O)—O—; triazolylene, and any combination thereof.

38. The delivery system of claim 33, wherein the hydrophobic segments of the di- and/or tri-block copolymer comprise hydrophobic dendrons comprising between 0 to 5 generations or between 0 to 3 generations.

39. The delivery system of claim 38, wherein each generation of the hydrophobic dendron comprises a linear or branched C₁-C₂₀ alkylene, C₂-C₂₀ alkenylene, C₂-C₂₀ alkynylene or arylene moiety which is substituted at each end with a group selected from the group consisting of —O—, —S—, —NH—, —C(═O)—, —C(═O)—O—, —O—C(═O)—O—, —C(═O)—NH—, —NH—C(═O)—NH—, —NH—C(═O)—O—, —S(═O)—, —S(═O)—O—, —PO(═O)—O—, and any combination thereof; or wherein each generation of the hydrophobic dendron is derived from a compound having the following structure HX—Z—XH or HX—Z—CO₂H, wherein X is independently at each occurrence NH, S or O, and Z is selected from C₁-C₁₀ alkylene, C₂-C₁₀ alkenylene, C₂-C₁₀ alkynylene, and arylene.

40. The delivery system of claim 38, wherein each generation of the dendron is derived from a compound selected from the group consisting of HX—CH₂—CH₂—XH, HX—(CH₂)₁₋₃—CO₂H, and HX—CH₂—CH(XH)—CH₂—XH, wherein X is independently at each occurrence NH, S or O, or wherein each generation of the dendron is derived from a compound selected from the group consisting of HS—CH₂—CH₂—OH, HS—(CH₂)₁₋₃—CO₂H and HS—CH₂—CH(OH)—CH₂—OH.

41. The delivery system of claim 33, wherein the first and/or second stimulus is chemically-induced or physically-induced.

42. The delivery system of claim 33, wherein the first and/or second stimulus comprises the addition of a transport protein or an activating enzyme.

43. The delivery system of claim 42, wherein the transport protein is a serum albumin or wherein the activating enzyme is selected from the group consisting of an amidase, an esterase, and a urease.

44. The delivery system of claim 33, wherein the first and/or second stimulus-responsive cleavable site comprises a cleavable bond selected from the group consisting of a disulfide, a diselenide, an anhydride, an ester, an amide, an amidine, an imine, a carbamate, a carbonate, an acetal, a urea, a thiourea, a trithionate, a sulfate, a sulfamate, a phosphate, a phosphoamide, a hydrazone, an ether, a silyl ether, an oxyme, a boronic acid, a nitro, and an azo.

45. The delivery system of claim 44, wherein the first and/or second stimulus-responsive cleavable site comprises a functional group represented by the structure of —O—C(O)—R′, —C(O)—OR′—NH—C(O)—R′ or —C(O)—NHR′, wherein R′ is C₁-C₁₂ alkyl or an aryl.

46. The delivery system of claim 33, wherein the mole ratio of the di-block copolymer to the tri-block copolymer is about 1:10 to about 10:1.

47. The delivery system of claim 46, wherein the mole ratio of the di-block copolymer to the tri-block copolymer is about 1:1, about 2:1 or about 3:2.

48. The delivery system of claim 33, wherein the di- and tri-block copolymers are represented by the structures depicted in any one of FIGS. 2A, 17, and 39.

49. The delivery system of claim 33, wherein the hydrophilic to lipophilic balance (HLB) of the di-block copolymer is substantially identical to the HLB of the tri-block copolymer.

50. The delivery system of claim 33 further comprising a cargo, wherein the cargo is encapsulated within the micelles, covalently linked to the hydrophobic segment of the di-block copolymer, or covalently linked to the hydrophobic segments of the tri-block copolymer.

51. The delivery system of claim 50, wherein the cargo is selected from the group consisting of a pharmaceutical active ingredient, an agrochemical agent, a cosmetic agent, an imaging agent, and a diagnostic agent.

52. A method of delivering a cargo to a target site, the method comprising:
providing a delivery system comprising a mixture of a di-block copolymer and a tri-block copolymer, wherein the di-block copolymer comprises a hydrophilic segment and a hydrophobic segment comprising a first stimulus-responsive cleavable site, and the tri-block copolymer comprises two substantially identical hydrophobic segments each comprising a second stimulus-responsive cleavable site and a hydrophilic segment therebetween, and a cargo encapsulated within or attached thereto, wherein the delivery system is in the form of self-assembled micelles or fibers which transform to micelles upon hydration;
applying a first stimulus to induce cleavage of the first stimulus-responsive cleavable site thereby invoking a first phase transition from micelles to a hydrogel; and
applying a second stimulus to induce cleavage of the second stimulus-responsive cleavable sites and disassembly of the hydrogel thereby invoking a second phase transition to dissolved polymers and fragments thereof and releasing the cargo at the target site.
